(19) United States
(12) Patent Application Publication
(10) Pub. No.: US 2025/0289822 A1
(43) Pub. Date: Sep. 18, 2025

(54) SOLID FORMS OF HIV INTEGRASE INHIBITORS

(71) Applicant: Gilead Sciences, Inc., Foster City, CA (US)

(72) Inventors: Peter C. Fung, San Mateo, CA (US); Xiaochun Han, San Jose, CA (US); Lucas Man, Burlingame, CA (US); Henry G. Morrison, Dublin, CA (US)

(21) Appl. No.: 19/066,332

(22) Filed: Feb. 28, 2025

Related U.S. Application Data
(60) Provisional application No. 63/560,257, filed on Mar. 1, 2024.

(51) Int. Cl. C07D 471/18

(52) U.S. Cl. CPC C07D 471/18

(57) ABSTRACT

The present disclosure relates generally to solid forms of the compound of Formula I:

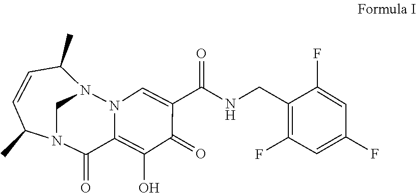

Also disclosed are pharmaceutical compositions comprising said solid forms and methods of making said solid forms. The solid forms of the disclosure are useful in treating or preventing human immunodeficiency virus (HIV) infection.

### CROSS-REFERENCE TO RELATED APPLICATIONS

This application claims priority to U.S. Provisional Application No. 63/560,257, filed Mar. 1, 2024. The entire contents of this application are hereby incorporated by reference in their entirety.

### TECHNICAL FIELD

The present disclosure relates to solid forms of the HIV integrase inhibitor (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide, for use in the treatment of HIV infections. The present disclosure also relates to pharmaceutical compositions containing the solid forms disclosed herein, and methods of treating or preventing HIV viral infections.

### BACKGROUND

There is an ongoing need for antiviral agents and methods for treating HIV viral infections. There is also a constant need to develop methods for preparation and purification of the antiviral agents, as well as prepare improved pharmaceutical formulations of the same. The solid forms disclosed herein help meet these and other needs.

### SUMMARY

The present disclosure provides crystalline forms of the compound of Formula I:

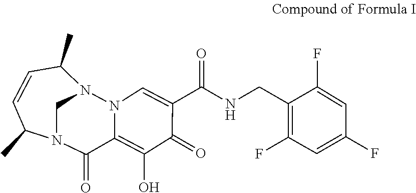

(1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide.

The present disclosure also provides salts of the compound of Formula I, as well as crystalline forms of the salts.

The present disclosure also provides co-crystals of the compound of Formula I, as well as crystalline forms of the co-crystals.

The present disclosure further provides a pharmaceutical composition comprising a crystalline form, amorphous form, salt, co-crystal of the compound of Formula I disclosed herein, and a pharmaceutically acceptable excipient.

The present disclosure further provides a kit comprising a crystalline form, amorphous form, salt, co-crystal of the compound of Formula I disclosed herein, and a pharmaceutically acceptable excipient.

The present disclosure further provides methods of treating an HIV infection in a human having or at risk of having the infection, comprising administering to the human a therapeutically effective amount of a crystalline form, amorphous form, salt, or co-crystal disclosed herein.

### BRIEF DESCRIPTION OF THE DRAWINGS

FIG. 1. Shows the XRPD pattern for the compound of Formula I, Form I.

FIG. 2. Shows the DSC curve for the compound of Formula I, Form I

FIG. 3. Shows the TGA curve for the compound of Formula I, Form I.

FIG. 4. Shows the DVS curve for the compound of Formula I, Form I.

FIG. 5. Shows the ball and stick drawing of the compound of Formula I, Form I.

FIG. 6. Shows the XRPD pattern for the compound of Formula I, Form II.

FIG. 7. Shows the DSC curve for the compound of Formula I, Form II.

FIG. 8. Shows the TGA curve for the compound of Formula I, Form II.

FIG. 9. Shows the XRPD pattern for the compound of Formula I, Sodium Salt, Form I.

FIG. 10. Shows the DSC curve for the compound of Formula I, Sodium Salt, Form I.

FIG. 11. Shows the TGA curve for the compound of Formula I, Sodium Salt, Form I.

FIG. 12. Shows the DVS curve for the compound of Formula I, Sodium Salt Form I.

FIG. 13. Shows the XRPD pattern for the compound of Formula I, Sodium Salt, Form II.

FIG. 14. Shows the DSC curve for the compound of Formula I, Sodium Salt, Form II.

FIG. 15. Shows the TGA curve for the compound of Formula I, Sodium Salt, Form II.

FIG. 16. Shows the XRPD pattern for the compound of Formula I, Sodium Salt, Form III.

FIG. 17. Shows the XRPD pattern for the compound of Formula I, Sodium Salt, Form IV.

FIG. 18. Shows the XRPD pattern for the compound of Formula I, Potassium Salt, Form I.

FIG. 19. Shows the DSC curve for the compound of Formula I, Potassium Salt, Form I.

FIG. 20. Shows the TGA curve for the compound of Formula I, Potassium Salt, Form I.

FIG. 21. Shows the DVS curve for the compound of Potassium Salt, Form I.

FIG. 22. Shows the atomic displacement ellipsoid drawing of the Compound of Formula I, Potassium Salt, Form I.

FIG. 23. Shows the XRPD pattern for the compound of Formula I, Potassium Salt, Form II.

FIG. 24. Shows the TGA curve for the compound of Formula I, Potassium Salt, Form II.

FIG. 25. Shows the XRPD pattern for the compound of Formula I, Potassium Salt, Form III.

FIG. 26. Shows the XRPD pattern for the compound of Formula I, Diethylamine Salt, Form I.

FIG. 27. Shows the DSC curve for the compound of Formula I, Diethylamine Salt, Form I.

FIG. 28. Shows the TGA curve for the compound of Formula I, Diethylamine Salt, Form II.

FIG. 29. Shows the DVS curve for the compound of Formula I, Diethylamine Salt, Form II.

FIG. 30. Shows the XRPD pattern for the compound of Formula I, Ammonia Salt.

FIG. 31. Shows the DSC curve for the compound of Formula I, Ammonia Salt.

FIG. 32. Shows the TGA curve for the compound of Formula I, Ammonia Salt.

FIG. 33. Shows the DVS curve for the compound of Formula I, Ammonia Salt.

FIG. 34. Shows the XRPD pattern for the compound of Formula I, Calcium Salt, Form I.

FIG. 35. Shows the DSC curve for the compound of Formula I, Calcium Salt, Form I.

FIG. 36. Shows the XRPD pattern for the compound of Formula I, Calcium Salt, Form II.

FIG. 37. Shows the XRPD pattern for the compound of Formula I, Calcium Salt, Form III.

FIG. 38. Shows the XRPD pattern for the compound of Formula I, Magnesium Salt, Form I.

FIG. 39. Shows the DSC curve for the compound of Formula I, Magnesium Salt, Form I.

FIG. 40. Shows the XRPD pattern for the compound of Formula I, Magnesium Salt, Form II.

FIG. 41. Shows the XRPD pattern for the compound of Formula I, N-Butylamine Salt.

FIG. 42. Shows the DSC curve for the compound of Formula I, N-Butylamine Salt.

FIG. 43. Shows the TGA curve for the compound of Formula I, N-Butylamine Salt.

FIG. 44. Shows the DVS curve for the compound of Formula I, N-Butylamine Salt.

FIG. 45. Shows the XRPD pattern for the compound of Formula I, diethanolamine salt.

FIG. 46. Shows the XRPD pattern for the compound of Formula I, ethylenediamine salt.

FIG. 47. Shows the XRPD pattern for the compound of Formula I, morpholine salt.

FIG. 48. Shows the XRPD pattern for the compound of Formula I, L-Arginine Salt, Form I.

FIG. 49. Shows the XRPD pattern for the compound of Formula I, L-Arginine Salt, Form II.

FIG. 50. Shows the XRPD pattern for the compound of Formula I, L-Arginine Salt, Form III.

FIG. 51. Shows the XRPD pattern for the compound of Formula I, trans-Ferulic Cocrystal, Form I.

FIG. 52. Shows the DSC curve for the compound of Formula I, trans-Ferulic Cocrystal, Form I.

FIG. 53. Shows the TGA curve for the compound of Formula I, trans-Ferulic Cocrystal, Form I.

FIG. 54. Shows the DVS curve for the compound of Formula I, trans-Ferulic Cocrystal, Form I.

FIG. 55. Shows the XRPD pattern for the compound of Formula I, trans-Ferulic Cocrystal, Form II.

FIG. 56. Shows the DSC curve for the compound of Formula I, trans-Ferulic co-crystal, Form II.

FIG. 57. Shows the TGA curve for the compound of Formula I, trans-Ferulic co-crystal, Form II.

FIG. 58. Shows the XRPD pattern for the compound of Formula I, Tromethamine Cocrystal.

FIG. 59. Shows the DSC curve for the compound of Formula I, Tromethamine Cocrystal.

FIG. 60. Shows the TGA curve for the compound of Formula I, Tromethamine Cocrystal.

### DETAILED DESCRIPTION

The present invention relates to new solid forms of the HIV integrase inhibitor (1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (Compound of Formula I, see below), which was disclosed in WO 2022/159387.

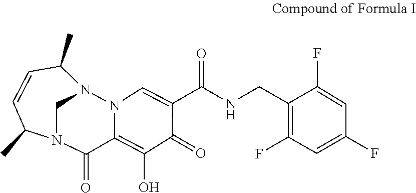

The solid forms of the invention include salt forms (both amorphous and crystalline) as well as co-crystal forms of the compound of Formula I. As used herein, “solid form” generally refers to a solid chemical substance that can be in any morphological form such as, for example, crystalline or amorphous, as well as disordered crystals, liquid crystals, plastic crystals, mesophases, and the like, or any combination thereof. In some embodiments, the solid form of the invention is a salt of the compound of Formula I which can be amorphous or crystalline. In further embodiments, the solid form can be a co-crystal of the compound of Formula I, in which the compound of Formula I has formed a crystalline solid together with a coformer molecule. Both crystalline salts and co-crystals of the compound of Formula I can exist in different crystalline forms (i.e., have different polymorphic or pseudopolymorphic forms).

As used herein, the term “co-crystal” or “cocrystal” refers to a compound (such as the compound of Formula I) crystallized together with one or more coformer molecules (e.g., molecules other than the compound). Depending on the chemical nature and proportion of coformers present in the co-crystal, different physical properties related to, for example, dissolution and solubility may be observed compared with solid forms of the compound by itself or salts thereof. In some instances, the coformer molecule may be a protic acid, and whether the protic acid forms a salt or a co-crystal will often depend on the relative pKa's of the compound and coformer. See, e.g., Regulatory Classification of Pharmaceutical Co-Crystals: Guidance for Industry, February 2018, published by the U.S. Dept. of Health and Human Services, FDA, Center for Drug Evaluation and Research (CDER).

As used herein, “crystalline form” is meant to refer to a certain lattice configuration of a crystalline substance (e.g., a salt or a co-crystal). Different crystalline forms of the same substance typically have different crystalline lattices (e.g., unit cells) which are attributed to different physical properties that are characteristic of each of the crystalline forms. In some instances, different lattice configurations have different water or solvent content giving rise to solvated or hydrated crystalline forms. The term “solvated,” as used herein, is meant to refer to a crystalline form that includes solvent molecules in the crystalline lattice. The term “hydrated,” as used herein, is meant to refer to a crystalline form that is solvated, where the solvent is water and water molecules are included in the crystalline lattice. Example “hydrated” crystalline forms include hemihydrates, monohydrates, dihydrates, and the like. Other hydrated forms such as channel hydrates and the like are also included within the meaning of the term. The term “fully hydrated” is meant to refer to where the water content of the hydrate is present in the expected stoichiometric amounts. The term “partially hydrated” is meant to refer to where the water content of the hydrate is present in less than the expected stoichiometric amounts (e.g., where some of the water of a monohydrate has been removed). Similarly, the term “unsolvated” or “anhydrous” refers to a crystalline form being substantially free of solvent or water, respectively, although some residual solvent or water may be present, for example, left over from the processes used to prepare the crystalline form.

According to the present invention, a crystalline form of a salt or co-crystal of the compound of Formula I can be useful in the synthesis and/or purification of the compound of Formula I. For example, a crystalline form of a salt or co-crystal of the compound of Formula I can be an intermediate in the synthesis of the compound of Formula I. In addition, different crystalline forms of salts and co-crystals of the compound of Formula I may have different properties with respect to bioavailability, stability, purity, and/or manufacturability for medical or pharmaceutical uses. Variations in the crystal structure of a pharmaceutical drug substance or active ingredient may affect the dissolution rate (which may affect bioavailability, etc.), manufacturability (e.g., ease of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, the crystalline forms of the salts and co-crystals of the compound of Formula I may provide advantages such as improving the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different crystalline forms of the salts and co-crystals of the compound of Formula I which may exhibit one or more of the favorable characteristics described above. The processes for the preparation of the crystalline and co-crystal forms described herein and characterization of these crystalline and co-crystal forms are described in detail below.

In some embodiments, the pharmaceutically acceptable salts described herein, co-crystals, or crystalline forms thereof, are purified or substantially isolated. By “substantially isolated” it is meant that the salt, co-crystal, or crystalline form thereof is at least partially or substantially separated from the environment in which it was formed or detected. Partial separation can include, for example, a composition enriched in the salt, co-crystal, or crystalline form of the invention. Substantial separation can include compositions containing at least about 50%, at least about 60%, at least about 70%, at least about 80%, at least about 90%, at least about 95%, at least about 97%, or at least about 99% by weight of the salt, co-crystal, or crystalline form of the invention. In some embodiments, the salt, co-crystal, or crystalline form of the invention can be prepared with a purity of about 75% or more, 80% or more, 85% or more, 90% or more, 95% or more, 98% or more, or 99% or more.

The different crystalline forms can be identified by solid state characterization methods such as by X-ray powder diffraction (XRPD). Other characterization methods such as differential scanning calorimetry (DSC), thermogravimetric analysis (TGA), and dynamic vapor sorption (DVS) further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

The present invention provides crystalline forms of certain compounds or salts thereof. In some embodiments, the crystalline form may be substantially anhydrous. In some embodiments, the crystalline form may be hydrated or solvated.

### Compound of Formula I, Form I

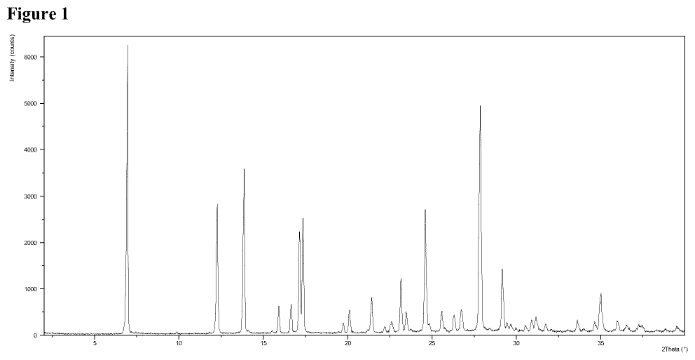

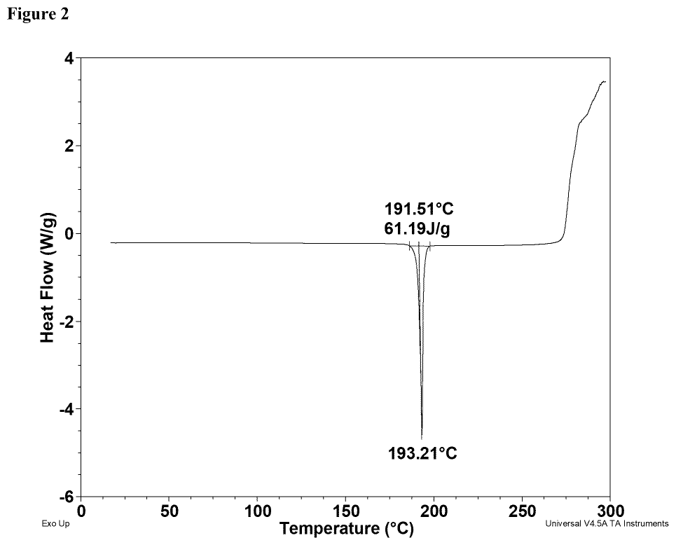

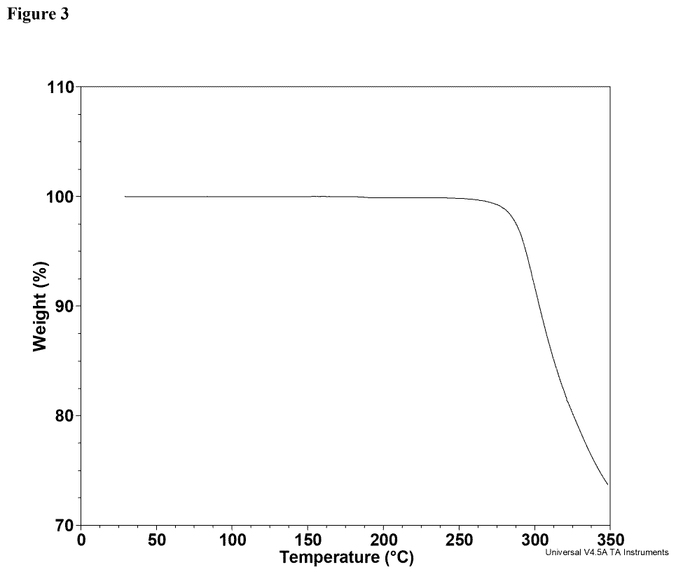

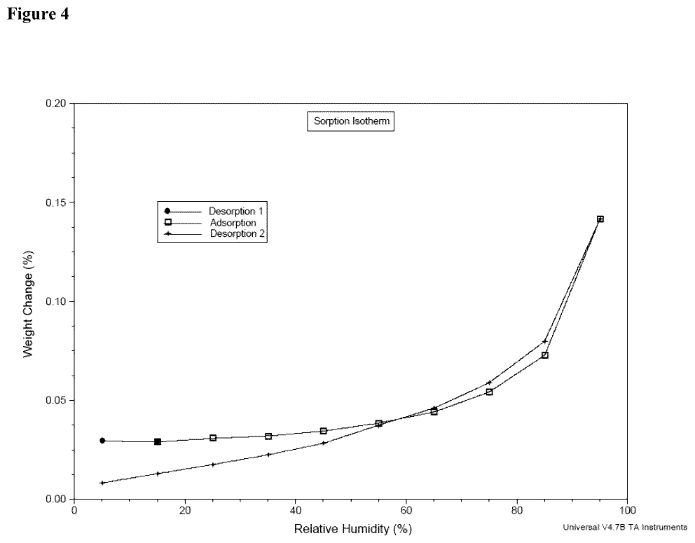

In some embodiments, the present disclosure provides a crystalline Form I of the compound of Formula I (“Formula I, Form I” or “compound of Formula I, Form I”). In some embodiments, the crystal structure of the compound of Formula I, Form I exhibits an XRPD pattern substantially as shown in FIG. 1. Formula I, Form I may exhibit a DSC thermogram substantially as shown in FIG. 2. Formula I, Form I may exhibit a TGA graph substantially as shown in FIG. 3. Formula I, Form I may exhibit a DVS curve substantially as shown in FIG. 4.

In some embodiments of Formula I, Form I, at least one, at least two, at least three, or all of the following (a)-(d) apply: (a) Formula I, Form I has an XRPD pattern substantially as shown in FIG. 1; (b) Formula I, Form I has a DSC thermogram substantially as shown in FIG. 2; (c) Formula I, Form I has a TGA curve substantially as shown in FIG. 3; (d) Formula I, Form I has a DVS curve substantially as shown in FIG. 4.

In some embodiments, Formula I, Form I has the following properties:
(a) an XRPD pattern substantially as shown in FIG. 1;
(b) a DSC thermogram substantially as shown in FIG. 2;
(c) a TGA curve substantially as shown in FIG. 3; and
(d) a DVS curve substantially as shown in FIG. 4.

In some embodiments, Formula I, Form I has an XRPD pattern displaying at least two, at least three, at least four, at least five, or at least six of the degree 2θ-reflections with the greatest intensity as the XRPD pattern substantially as shown in FIG. 1.

In some embodiments, Formula I, Form I has an XRPD pattern comprising 2θ-reflections (+/−0.2 degrees 2θ) at 7.0°, 13.9°, and 27.9°. In some embodiments, Formula I, Form I has an XRPD pattern comprising 2θ-reflections (+/−0.2 degrees 2θ) at 7.0°, 13.9°, and 27.9°, and one, two or three of the 2θ-reflections (+/−0.2 degrees 2θ) at 12.3°, 17.4°, and 24.6°. In some embodiments, Formula I, Form I has an XRPD pattern comprising 2θ-reflections (+/−0.2 degrees 2θ) at 7.0°, 13.9°, and 27.9°, and one or two of the 2θ-reflections (+/−0.2 degrees 2θ) at 12.3°, 17.4°, and 24.6°. In some embodiments, Formula I, Form I has an XRPD pattern comprising 2θ-reflections (+/−0.2 degrees 2θ) at 7.0°, 13.9°, and 27.9°, and one of the 2θ-reflections (+/−0.2 degrees 2θ) at 12.3°, 17.4°, and 24.6°. In some embodiments, Formula I, Form I has an XRPD pattern comprising 2θ-reflections (+/−0.2 degrees 2θ) at 7.0°, 13.9°, and 27.9°, and two of the 2θ-reflections (+/−0.2 degrees 2θ) at 12.3°, 17.4°, and 24.6°. In some embodiments, Formula I, Form I has an XRPD pattern comprising 2θ-reflections (+/−0.2 degrees 2θ) at 7.0°, 12.3°, 13.9°, 17.4°, and 24.6°, and 27.9°. In some embodiments, Formula I, Form I has an XRPD pattern comprising any three 2θ-reflections (+/−0.2 degrees 2θ) selected from the group consisting of 7.0°, 12.3°, 13.9°, 17.4°, 24.6°, and 27.9°.

In some embodiments, Formula I, Form I has an XRPD pattern comprising 2θ-reflections (+/−0.2 degrees 2θ) at 7.0°, 12.3°, 13.9°, 17.4°, 24.6°, and 27.9°, and one, two, or three of the 2θ-reflections (+/−0.2 degrees 2θ) at 21.4°, 23.2°, and 29.2°. In some embodiments, Formula I, Form I has an XRPD pattern comprising 2θ-reflections (+/−0.2 degrees 2θ) at 7.0°, 12.3°, 13.9°, 17.4°, 24.6°, and 27.9°, and one or two of the 2θ-reflections (+/−0.2 degrees 2θ) at 21.4°, 23.2°, and 29.2°. In some embodiments, Formula I, Form I has an XRPD pattern comprising 2θ-reflections (+/−0.2 degrees 2θ) at 7.0°, 12.3°, 13.9°, 17.4°, 24.6°, and 27.9°, and one of the 20-reflections (+/−0.2 degrees 2θ) at 21.4°, 23.2°, and 29.2°. In some embodiments, Formula I, Form I has an XRPD pattern comprising 2θ-reflections (+/−0.2 degrees 2θ) at 7.0°, 12.3°, 13.9°, 17.4°, 24.6°, and 27.9°, and two of the 2θ-reflections (+/−0.2 degrees 2θ) at 21.4°, 23.2°, and 29.2°. In some embodiments, Formula I, Form I has an XRPD pattern comprising 2θ-reflections (+/−0.2 degrees 2θ) at 7.0°, 12.3°, 13.9°, 17.4°, 21.4°, 23.2°, 24.6°, 27.9°, and 29.2°. In some embodiments, Formula I, Form I has an XRPD pattern comprising three of the 2θ-reflections (+/−0.2 degrees 2θ) at 7.0°, 12.3°, 13.9°, 17.4°, 21.4°, 23.2°, 24.6°, 27.9°, and 29.2°. In some embodiments, Formula I, Form I has an XRPD pattern comprising at least three of the 20-reflections (+/−0.2 degrees 2θ) at 7.0°, 12.3°, 13.9°, 17.4°, 21.4°, 23.2°, 24.6°, 27.9°, and 29.2°. In some embodiments, Formula I, Form I has an XRPD pattern comprising at least four of the 2θ-reflections (+/−0.2 degrees 2θ) at 7.0°, 12.3°, 13.9°, 17.4°, 21.4°, 23.2°, 24.6°, 27.9°, and 29.2°. In some embodiments, Formula I, Form I has an XRPD pattern comprising at least five of the 2θ-reflections (+/−0.2 degrees 2θ) at 7.0°, 12.3°, 13.9°, 17.4°, 21.4°, 23.2°, 24.6°, 27.9°, and 29.2°.

In some embodiments, Formula I, Form I has an XRPD pattern comprising at least three of the 2θ-reflections (+/−0.2 degrees 2θ) at 7.0°, 12.3°, 13.9°, 17.1°, 17.4°, 21.4°, 23.2°, 24.6°, 27.8°, 27.9°, and 29.2°. In some embodiments, Formula I, Form I has an XRPD pattern comprising at least four of the 2θ-reflections (+/−0.2 degrees 2θ) at 7.0°, 12.3°, 13.9°, 17.1°, 17.4°, 21.4°, 23.2°, 24.6°, 27.8°, 27.9°, and 29.2°. In some embodiments, Formula I, Form I has an XRPD pattern comprising at least five of the 2θ-reflections (+/−0.2 degrees 2θ) at 7.0°, 12.3°, 13.9°, 17.1°, 17.4°, 21.4°, 23.2°, 24.6°, 27.8°, 27.9°, and 29.2°. In some embodiments, Formula I, Form I has an XRPD pattern comprising 2θ-reflections (+/−0.2 degrees 2θ) at 7.0°, 12.3°, 13.9°, 17.1°, 17.4°, 21.4°, 23.2°, 24.6°, 27.8°, 27.9°, and 29.2°.

In some embodiments, Formula I, Form I has an XRPD pattern comprising peaks at:

| Pos. | Rel. Int. |
|---|---|
| [°2Th.] | [%] |
| 7.0 | 100 |
| 12.3 | 44 |
| 13.9 | 57 |
| 15.9 | 9 |
| 16.7 | 10 |
| 17.1 | 34 |
| 17.4 | 40 |
| 20.1 | 8 |
| 21.4 | 12 |
| 22.6 | 4 |
| 23.2 | 18 |
| 24.6 | 41 |
| 25.6 | 7 |
| 26.3 | 5 |
| 26.7 | 7 |
| 27.8 | 42 |
| 27.9 | 76 |
| 29.2 | 20 |
| 31.2 | 4 |
| 35.0 | 12 |
| 36.6 | 2 |
| 37.4 | 2 |

In some embodiments, Formula I, Form I, is characterized by a DSC curve comprising an endothermic transition with an onset at about 192° C. In some embodiments, Formula I, Form I, is characterized by a DSC curve substantially as shown in FIG. 2.

In some embodiments, Formula I, Form I is unsolvated. In some embodiments, Formula I, Form I, is characterized by a TGA curve substantially as shown in FIG. 3.

In some embodiments, Formula I, Form I, is characterized by a DVS curve substantially as shown in FIG. 4. In some embodiments, Formula I, Form I, absorbs about 0.15% of water up to 95% RH at 25° C.

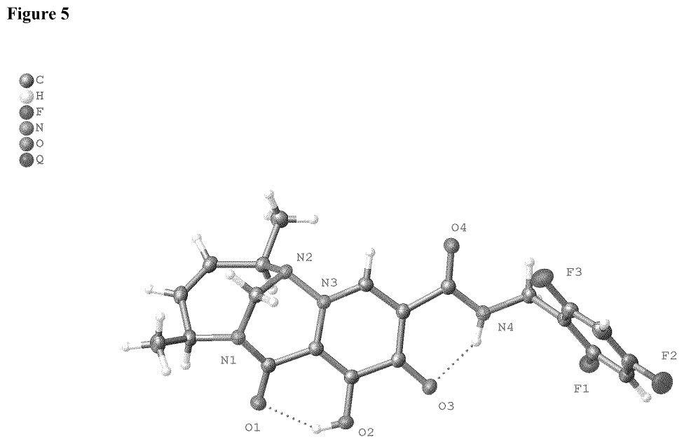

The single crystal data collected on Formula I, Form I are summarized in Table 1 below and also shown in FIG. 5. The crystal system of Formula I, Form I is monoclinic and the space group is P2₁. The cell parameters and calculated volume are: a=8.9146(2) Å, b=8.6717(2) Å, c=12.7101(3) Å, α=90°, β=93.3170(10)°, γ=90°, V=980.95(4) Å³. The molecular weight is 448.40 g mol⁻¹ with Z=2, resulting in a calculated density of 1.518 g cm⁻³.

TABLE 1
Crystal Data and Data Collection Parameters for Formula I, Form I

| | |
|---|---|
| Empirical formula | C₂₁H₁₉F₃N₄O₄ |
| Formula weight (g mol⁻¹) | 448.40 |
| Temperature (K) | 100 |
| Wavelength (Å) | 1.54184 |
| Crystal system | monoclinic |
| Space group | P2₁ |
| Unit cell parameters | |
| a = 8.9146(2) Å | α = 90° |
| b = 8.6717(2) Å | β = 93.3170(10)° |
| c = 12.7107(3) Å | γ = 90° |
| Unit cell volume (Å³) | 980.95(4) |
| Cell formula units, Z | 2 |
| Calculated density (g cm⁻³) | 1.518 |
| Absorption coefficient (mm⁻¹) | 1.081 |
| F(000) | 464 |
| Crystal size (mm³) | 0.23 × 0.18 × 0.16 |
| Total reflections collected | 18571 |
| Index ranges | −10 ≤ h ≤ 10; −10 ≤ k ≤ 10; −15 ≤ l ≤ 15 |
| θ range for data collection | θₘᵢₙ = 3.483°, θₘₐₓ = 71.009° |
| Completeness to θ_{full} = 67.679° | 100.0% |
| Absorption correction | semi-empirical form equivalents |
| Max. and min. transmission | 0.7534 and 0.6029 |
| Refinement method | full matrix least-squares on F² |
| Independent reflections | 3768 [Rᵢₙₜ = 0.0262] |
| Reflections/restraints/parameters | 3768/2/297 |
| Goodness-of-fit on F² | S = 1.028 |
| Final residuals [I > 2σ(I)] | R = 0.0224, R_{w} = 0.0603 |
| Final residuals [all reflections] | R = 0.0225, R_{w} = 0.0605 |
| Absolute structure parameter | 0.05(3) |
| Extinction coefficient | 0.0044(5) |
| Largest diff. peak and hole (eÅ−3) | 0.155 and −0.122 |

### Compound of Formula I, Form II

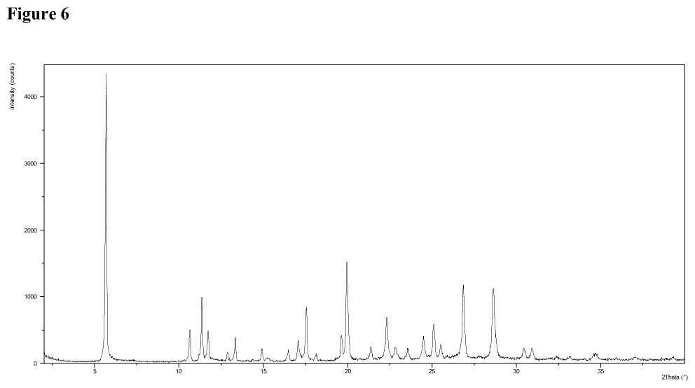

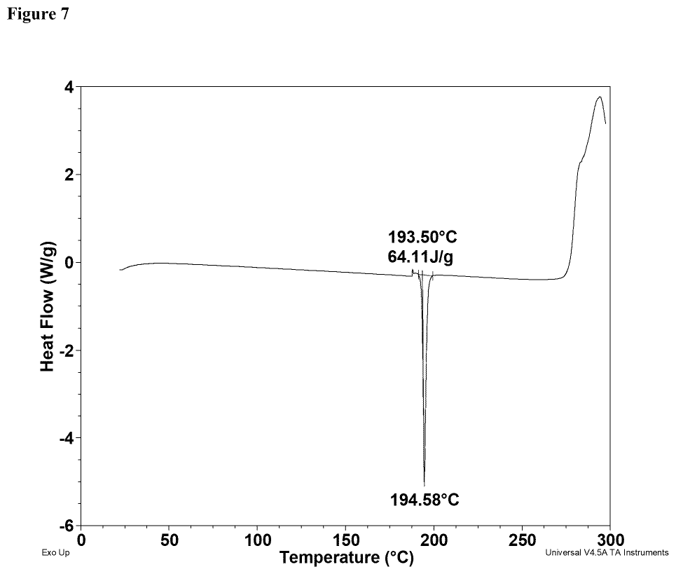

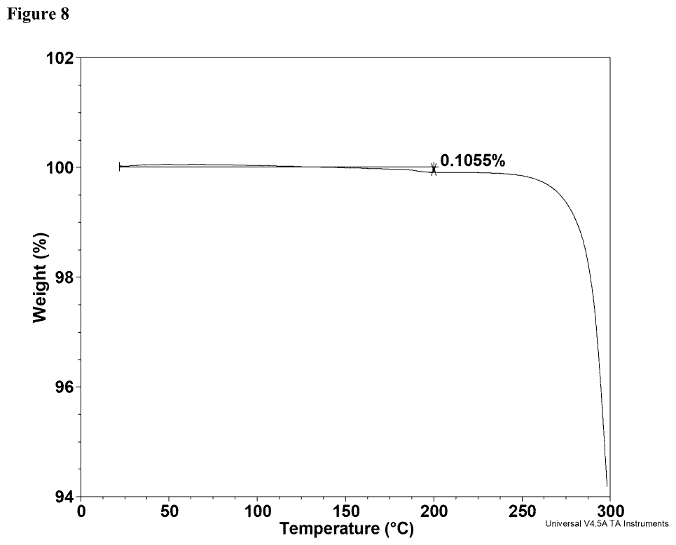

In some embodiments, provided here is crystalline Form II of the compound of Formula I (“Formula I, Form II” or “compound of Formula I, Form II”). In some embodiments, the crystal structure of the Compound of Formula I, Form II exhibits an XRPD pattern substantially as shown in FIG. 6. Formula I, Form II may exhibit a DSC thermogram substantially as shown in FIG. 7. Formula II, Form I may exhibit a TGA graph substantially as shown in FIG. 8.

In some embodiments of Formula I, Form II, at least one, at least two, at least three, or all of the following (a)-(c) apply: (a) Formula I, Form II has an XRPD pattern substantially as shown in FIG. 6; (b) Formula I, Form II has a DSC thermogram substantially as shown in FIG. 7; and (c) Formula I, Form II has a TGA curve substantially as shown in FIG. 8.

In some embodiments, Formula I, Form II has the following properties:
(a) an XRPD pattern substantially as shown in FIG. 6;
(b) a DSC thermogram substantially as shown in FIG. 7; and
(c) a TGA curve substantially as shown in FIG. 8.

In some embodiments, Formula I, Form II has an XRPD pattern displaying at least two, at least three, at least four, at least five, or at least six of the degree 2θ-reflections with the greatest intensity as the XRPD pattern substantially as shown in FIG. 6.

In some embodiments, Formula I, Form II has an XRPD pattern comprising 2θ-reflections (+/−0.2 degrees 2θ) at 5.7°, 19.9°, and 26.8°. In some embodiments, crystalline Formula I, Form II has an XRPD pattern comprising 2θ-reflections (+/−0.2 degrees 2θ) at 5.7°, 19.9°, and 26.8°, and one, two or three of the θ-reflections (+/−0.2 degrees 2θ) at 11.4°, 17.6°, and 28.6°. In some embodiments, crystalline Formula I, Form II has an XRPD pattern comprising 2θ-reflections (+/−0.2 degrees 2θ) at 5.7°, 19.9°, and 26.8°, and one or two of the 20-reflections (+/−0.2 degrees 2θ) at 11.4°, 17.6°, and 28.6°. In some embodiments, crystalline Formula I, Form II has an XRPD pattern comprising 2θ-reflections (+/−0.2 degrees 2θ) at 5.7°, 19.9°, and 26.8°, and one of the 2θ-reflections (+/−0.2 degrees 2θ) at 11.4°, 17.6°, and 28.6°. In some embodiments, crystalline Formula I, Form II has an XRPD pattern comprising 2θ-reflections (+/−0.2 degrees 2θ) at 5.7°, 19.9°, and 26.8°, and two of the 2θ-reflections (+/−0.2 degrees 2θ) at 11.4°, 17.6°, and 28.6°. In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°. In some embodiments, crystalline Formula I, Form II has an XRPD pattern comprising any three 2θ-reflections (+/−0.2 degrees 2θ) selected from the group consisting of 5.7°, 11.4°, 17.6°, 19.9°, 26.8°, and 28.6°.

In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°, and one, two or three of the 2θ-reflections (+/−0.2 degrees 2θ) at 10.7°, 22.3°, and 25.1°. In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°, and one or two of the 2θ-reflections (+/−0.2 degrees 2θ) at 10.7°, 22.3°, and 25.1°. In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°, and one of the 2θ-reflections (+/−0.2 degrees 2θ) at 10.7°, 22.3°, and 25.1°. In some embodiments, crystalline Formula I, Form II has an XRPD pattern comprising 2θ-reflections (+/−0.2 degrees 2θ) at 5.7°, 11.4°, 17.6°, 19.9°, 26.8°, and 28.6°, and two of the 20-reflections (+/−0.2 degrees 2θ) at 10.7°, 22.3°, and 25.1°. In some embodiments, crystalline Formula I, Form II has an XRPD pattern comprising any three 2θ-reflections (+/−0.2 degrees 2θ) selected from the group consisting of 5.7°, 10.7°, 11.4°, 17.6°, 19.9°, 22.3°, 25.10, 26.8°, and 28.6°. In some embodiments, crystalline Formula I, Form II has an XRPD pattern comprising 2θ-reflections (+/−0.2 degrees 2θ) at 5.7°, 10.7°, 11.4°, 17.6°, 19.9°, 22.3°, 25.1°, 26.8°, and 28.60.

In some embodiments, crystalline Formula I, Form II has an XRPD pattern comprising at least three of 2θ-reflections (+/−0.2 degrees 2θ) selected from the group consisting of 5.7°, 10.7°, 11.4°, 17.6°, 19.9°, 22.3°, 25.1°, 26.8°, and 28.6°. In some embodiments, crystalline Formula I, Form II has an XRPD pattern comprising at least four of 2θ-reflections (+/−0.2 degrees 2θ) selected from the group consisting of 5.7°, 10.7°, 11.4°, 17.6°, 19.9°, 22.3°, 25.1°, 26.8°, and 28.6°. In some embodiments, crystalline Formula I, Form II has an XRPD pattern comprising at least five of 2θ-reflections (+/−0.2 degrees 2θ) selected from the group consisting of 5.70, 10.70, 11.40, 17.60, 19.90, 22.30, 25.10, 26.80, and 28.60.

In some embodiments, crystalline Formula I, Form II has an XRPD pattern comprising at least three of 2θ-reflections (+/−0.2 degrees 2θ) selected from the group consisting of 5.7°, 10.7°, 11.4°, 11.7°, 17.6°, 19.9°, 22.3°, 25.1°, 26.8°, and 28.6°. In some embodiments, crystalline Formula I, Form II has an XRPD pattern comprising at least four of 2θ-reflections (+/−0.2 degrees 2θ) selected from the group consisting of 5.7°, 10.7°, 11.4°, 11.7°, 17.6°, 19.9°, 22.3°, 25.1°, 26.8°, and 28.6°. In some embodiments, crystalline Formula I, Form II has an XRPD pattern comprising at least five of 2θ-reflections (+/−0.2 degrees 2θ) selected from the group consisting of 5.7°, 10.7°, 11.4°, 11.7°, 17.6°, 19.9°, 22.3°, 25.1°, 26.8°, and 28.6°.

In some embodiments, Formula I, Form II has an XRPD pattern comprising peaks at:

| Pos. | Rel. Int. |
|---|---|
| [°2Th.] | [%] |
| 5.7 | 100 |
| 10.7 | 11 |
| 11.4 | 23 |
| 11.7 | 11 |
| 13.3 | 8 |
| 14.9 | 4 |
| 16.5 | 3 |
| 17.1 | 7 |
| 17.6 | 19 |
| 19.6 | 9 |
| 19.9 | 35 |
| 21.4 | 4 |
| 22.3 | 14 |
| 22.9 | 3 |
| 23.6 | 3 |
| 24.5 | 7 |
| 25.1 | 12 |
| 25.5 | 5 |
| 26.8 | 25 |
| 28.6 | 25 |
| 30.5 | 4 |
| 30.9 | 4 |
| 34.7 | 2 |

In some embodiments, Formula I, Form II, is characterized by a DSC curve comprising an endothermic transition with an onset at about 194° C. In some embodiments, Formula I, Form II, is characterized by a DSC curve substantially as shown in FIG. 7.

In some embodiments, Formula I, Form II is unsolvated. In some embodiments, Formula I, Form II, is characterized by a TGA curve substantially as shown in FIG. 8.

### Compound of Formula I, Sodium Salt

In some embodiments, the compound of Formula I can be isolated as a sodium salt which can be amorphous or crystalline. In some embodiments, the sodium salt of the compound of Formula I is crystalline.

### Compound of Formula I, Sodium Salt, Form I

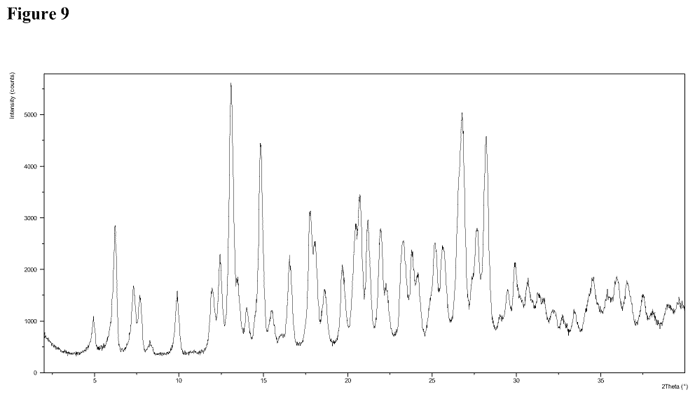

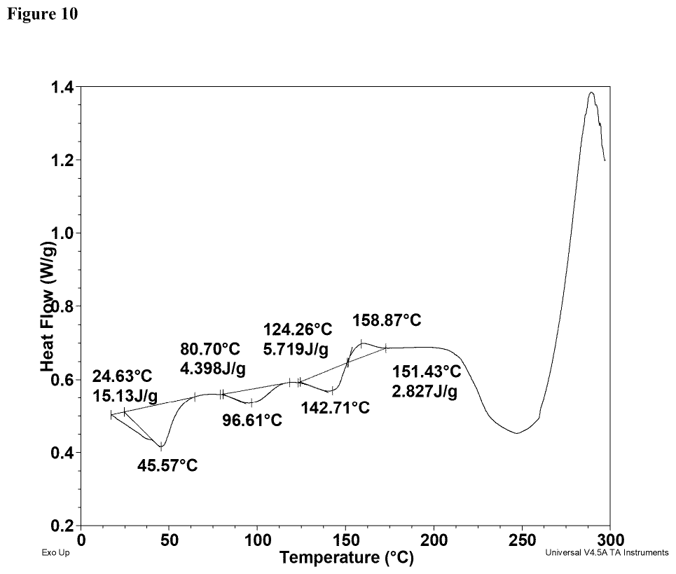

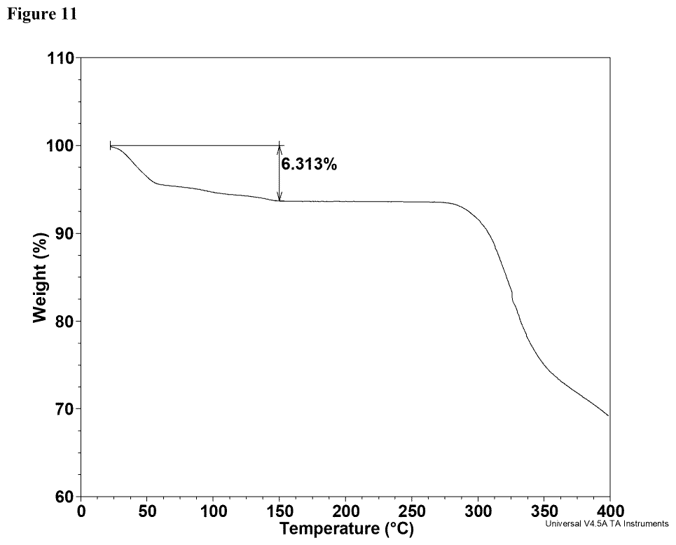

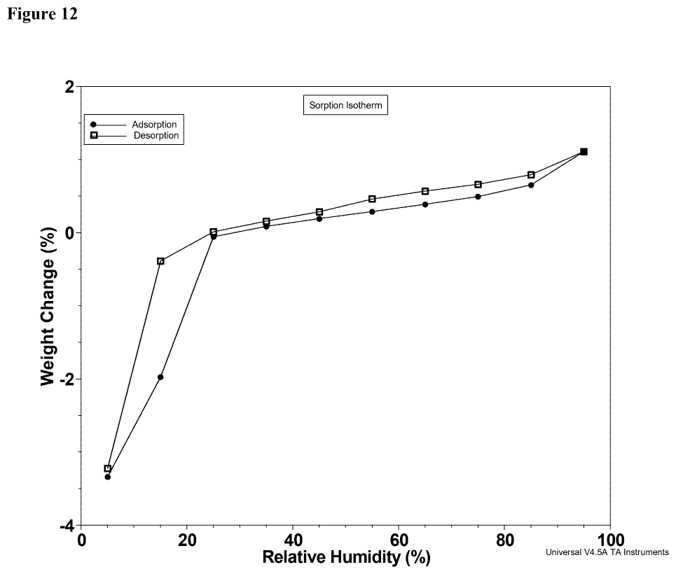

In some embodiments, the crystalline sodium salt of the compound of Formula I is a Form I (“compound of Formula I, sodium salt, Form I” or “Formula I, sodium salt, Form I”). In some embodiments, Compound of Formula I, sodium salt, Form I has an XRPD profile substantially as shown in FIG. 9. Compound of Formula I, sodium salt, Form I may exhibit a DSC thermogram substantially as shown in FIG. 10. Compound of Formula I, sodium salt, Form I may exhibit a TGA graph substantially as shown in FIG. 11. Compound of Formula I, sodium salt, Form I may exhibit a DVS curve substantially as shown in FIG. 12.

In some embodiments of Compound of Formula I, sodium salt, Form I, at least one, at least two, at least three, or all of the following (a)-(d) apply: (a) Compound of Formula I, sodium salt, Form I has an XRPD pattern substantially as shown in FIG. 9; (b) Compound of Formula I, sodium salt, Form I has a DSC thermogram substantially as shown in FIG. 10; (c) Compound of Formula I, sodium salt, Form I has a TGA curve substantially as shown in FIG. 11; (d) Compound of Formula I, sodium salt, Form I has a DVS curve substantially as shown in FIG. 12.

In some embodiments, Compound of Formula I, sodium salt, Form I has the following properties:
(a) an XRPD pattern substantially as shown in FIG. 9;
(b) a DSC thermogram substantially as shown in FIG. 10;
(c) a TGA curve substantially as shown in FIG. 11; and
(d) a DVS curve substantially as shown in FIG. 12.

In some embodiments, Formula I, sodium salt, Form I has an XRPD pattern displaying at least two, at least three, at least four, at least five, or at least six of the degree 2θ-reflections with the greatest intensity as the XRPD pattern substantially as shown in FIG. 9.

In some embodiments, the Formula I, sodium salt, Form I has an XRPD pattern comprising 2θ-reflections (+/−0.2 degrees 2θ) at 13.10, 14.8°, and 26.8°. In some embodiments, the Formula I, sodium salt, Form I has an XRPD pattern comprising 2θ-reflections (+/−0.2 degrees 2θ) at 13.10, 14.8°, and 26.8°, and one, two or three of the 2θ-reflections (+/−0.2 degrees 2θ) at 17.8°, 20.7°, and 28.2°. In some embodiments, the Formula I, sodium salt, Form I has an XRPD pattern comprising 2θ-reflections (+/−0.2 degrees 2θ) at 13.10, 14.8°, and 26.8°, and one or two of the 2θ-reflections (+/−0.2 degrees 2θ) at 17.8°, 20.7°, and 28.2°. In some embodiments, the Formula I, sodium salt, Form I has an XRPD pattern comprising 2θ-reflections (+/−0.2 degrees 2θ) at 13.10, 14.8°, and 26.8°, and one of the 2θ-reflections (+/−0.2 degrees 2θ) at 17.8°, 20.7°, and 28.2. In some embodiments, the Formula I, sodium salt, Form I has an XRPD pattern comprising 2θ-reflections (+/−0.2 degrees 2θ) at 13.10, 14.8°, and 26.8°, and two of the 2θ-reflections (+/−0.2 degrees 2θ) at 17.8°, 20.7°, and 28.2. In some embodiments, the Formula I, sodium salt, Form I has an XRPD pattern comprising 2θ-reflections (+/−0.2 degrees 2θ) at 13.10, 14.8°, 17.8°, 20.7°, 26.8°, and 28.2°. In some embodiments, the Formula I, sodium salt, Form I has an XRPD pattern comprising any three of the 2θ-reflections (+/−0.2 degrees 2θ) at 13.10, 14.8°, 17.8°, 20.7°, 26.8°, and 28.2°.

In some embodiments, the Formula I, sodium salt, Form I has an XRPD pattern comprising 2θ-reflections (+/−0.2 degrees 2θ) at 13.10, 14.8°, 17.8°, 20.7°, 26.8°, and 28.2°, and one, two or three of the 2θ-reflections (+/−0.2 degrees 2θ) at 6.2°, 21.2°, and 23.3°. In some embodiments, the Formula I, sodium salt, Form I has an XRPD pattern comprising 2θ-reflections (+/−0.2 degrees 2θ) at 13.10, 14.8°, 17.8°, 20.7°, 26.8°, and 28.2°, and one or two of the 20-reflections (+/−0.2 degrees 2θ) at 6.2°, 21.2°, and 23.3°. In some embodiments, the Formula I, sodium salt, Form I has an XRPD pattern comprising 2θ-reflections (+/−0.2 degrees 2θ) at 13.10, 14.8°, 17.8°, 20.7°, 26.8°, and 28.2°, and one of the 2θ-reflections (+/−0.2 degrees 2θ) at 6.2°, 21.2°, and 23.3°. In some embodiments, the Formula I, sodium salt, Form I has an XRPD pattern comprising 2θ-reflections (+/−0.2 degrees 2θ) at 13.10, 14.8°, 17.8°, 20.7°, 26.8°, and 28.2°, and two of the 2θ-reflections (+/−0.2 degrees 2θ) at 6.2°, 21.2°, and 23.3°. In some embodiments, the Formula I, sodium salt, Form I has an XRPD pattern comprising 2θ-reflections (+/−0.2 degrees 2θ) at 6.2°, 13.10, 14.8°, 17.8°, 20.7°, 21.2°, 23.3°, 26.8°, and 28.2°. In some embodiments, the Formula I, sodium salt, Form I has an XRPD pattern comprising any three of the 2θ-reflections (+/−0.2 degrees 2θ) at 6.2°, 13.10, 14.8°, 17.8°, 20.7°, 21.2°, 23.3°, 26.8°, and 28.2°.

In some embodiments, the Formula I, sodium salt, Form I has an XRPD pattern comprising at least three of the 2θ-reflections (+/−0.2 degrees 2θ) at 6.2°, 13.10, 14.8°, 17.8°, 20.7°, 21.2°, 23.3°, 26.8°, and 28.2°. In some embodiments, the Formula I, sodium salt, Form I has an XRPD pattern comprising at least four of the 2θ-reflections (+/−0.2 degrees 2θ) at 6.2°, 13.10, 14.8°, 17.8°, 20.7°, 21.2°, 23.3°, 26.8°, and 28.2°. In some embodiments, the Formula I, sodium salt, Form I has an XRPD pattern comprising at least five of the 2θ-reflections (+/−0.2 degrees 2θ) at 6.2°, 13.10, 14.8°, 17.8°, 20.7°, 21.2°, 23.3°, 26.8°, and 28.2°.

In some embodiments, the Formula I, sodium salt, Form I has an XRPD pattern comprising at least three of the 2θ-reflections (+/−0.2 degrees 2θ) at 6.2°, 12.4°, 13.10, 14.8°, 16.6°, 17.8°, 20.7°, 21.2°, 21.9°, 23.3°, 25.2°, 26.8°, and 28.2°. In some embodiments, the Formula I, sodium salt, Form I has an XRPD pattern comprising at least four of the 20-reflections (+/−0.2 degrees 2θ) at 6.2°, 12.4°, 13.10, 14.8°, 16.6°, 17.8°, 20.7°, 21.2°, 21.9°, 23.3°, 25.2°, 26.8°, and 28.2°. In some embodiments, the Formula I, sodium salt, Form I has an XRPD pattern comprising at least five of the 2θ-reflections (+/−0.2 degrees 2θ) at 6.2°, 12.4°, 13.1°, 14.8°, 16.6°, 17.8°, 20.7°, 21.2°, 21.9°, 23.3°, 25.2°, 26.8°, and 28.2°.

In some embodiments, the Formula I, sodium salt, Form I has an XRPD pattern comprising peaks at:

| Pos. | Rel. Int. |
|---|---|
| [°2Th.] | [%] |
| 4.9 | 11 |
| 6.2 | 47 |
| 7.7 | 21 |
| 9.9 | 22 |
| 11.9 | 22 |
| 12.4 | 34 |
| 13.1 | 100 |
| 14.8 | 76 |
| 16.6 | 32 |
| 17.8 | 50 |
| 18.7 | 20 |
| 19.7 | 27 |
| 20.7 | 54 |
| 21.2 | 43 |
| 21.9 | 40 |
| 23.3 | 35 |
| 25.2 | 32 |
| 25.6 | 28 |
| 26.8 | 79 |
| 28.2 | 73 |
| 29.9 | 23 |
| 36.6 | 15 |

In some embodiments, the Formula I, sodium salt, Form I is characterized by a DSC thermogram substantially as shown in FIG. 10.

In some embodiments, the Formula I, sodium salt, Form I is characterized by a DSC thermogram having one or more of (i) an endothermic transition at 25° C., (ii) an endothermic transition at 81° C., (iii) an endothermic transition at 124° C., and (iv) an exothermic transition at 151° C. In some embodiments, the Formula I, sodium salt, Form I is characterized by a DSC thermogram having (i) an endothermic transition at 25° C., (ii) an endothermic transition at 81° C., (iii) an endothermic transition at 124° C., and (iv) an exothermic transition at 151° C.

In some embodiments, the Formula I, sodium salt, Form I is characterized by a TGA curve substantially as shown in FIG. 11.

In some embodiments, the Formula I, sodium salt, Form I is characterized by a DVS curve substantially as shown in shown in FIG. 12.

In some embodiments, the Formula I, sodium salt, Form I absorbs about 4% of water up to 95% RH at 25° C.

### Compound of Formula I, Sodium Salt, Form II

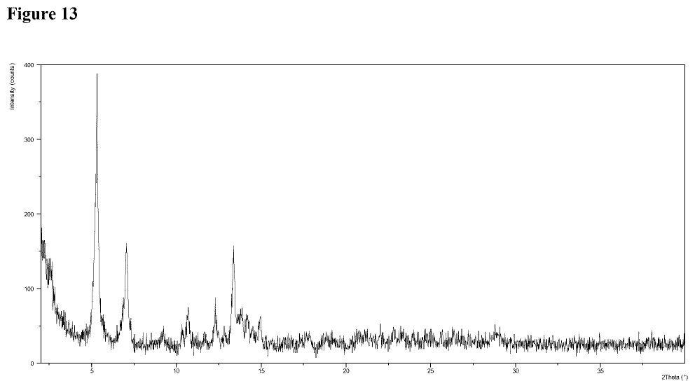

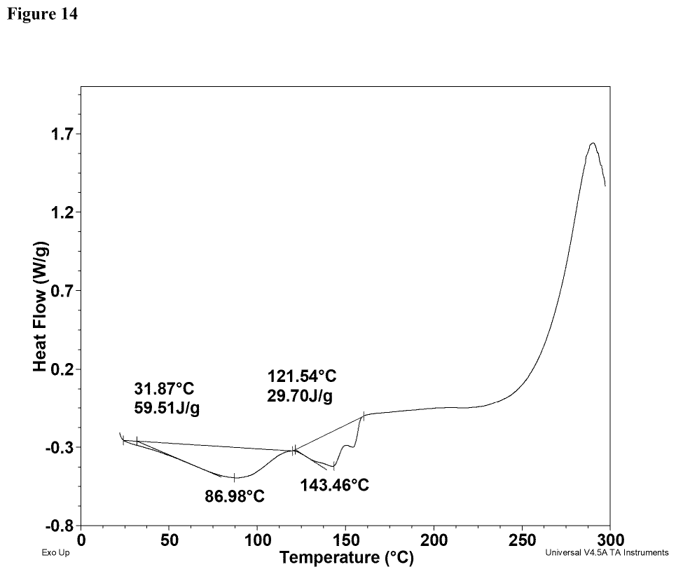

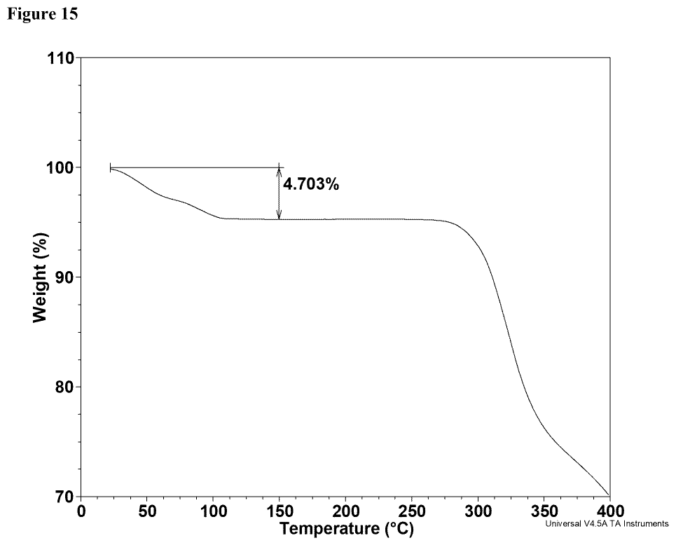

In some embodiments, the crystalline sodium salt of the compound of Formula I is a Form II (“compound of Formula I, sodium salt, Form II” or “Formula I, sodium salt, Form II”). In some embodiments, Compound of Formula I, sodium salt, Form II has an XRPD profile substantially as shown in FIG. 13. Compound of Formula I, sodium salt, Form II may exhibit a DSC thermogram substantially as shown in FIG. 14. Compound of Formula I, sodium salt, Form II may exhibit a TGA graph substantially as shown in FIG. 15.

In some embodiments of Compound of Formula I, sodium salt, Form II, at least one, at least two, at least three, or all of the following (a)-(c) apply: (a) Compound of Formula I, sodium salt, Form II has an XRPD pattern substantially as shown in FIG. 13; (b) Compound of Formula I, sodium salt, Form II has a DSC thermogram substantially as shown in FIG. 14; (c) Compound of Formula I, sodium salt, Form II has a TGA curve substantially as shown in FIG. 14.

In some embodiments, Compound of Formula I, sodium salt, Form II has the following properties:
(a) an XRPD pattern substantially as shown in FIG. 13;
(b) a DSC thermogram substantially as shown in FIG. 14; and
(c) a TGA curve substantially as shown in FIG. 15.

In some embodiments, the Formula I, sodium salt, Form II has an XRPD pattern comprising 2θ-reflections (+/−0.2 degrees 2θ) at 5.3°, 7.1°, and 13.4°. In some embodiments, the Formula I, sodium salt, Form II has an XRPD pattern comprising 2θ-reflections (+/−0.2 degrees 2θ) at 5.3°, 7.1°, 10.7°, and 13.4°.

In some embodiments, the Formula I, sodium salt, Form II has an XRPD pattern comprising peaks at:

| Pos. | Rel. Int. |
|---|---|
| [°2Th.] | [%] |
| 5.3 | 100 |
| 7.1 | 44 |
| 10.7 | 15 |
| 13.4 | 41 |

In some embodiments, the Formula I, sodium salt, Form II is characterized by a DSC thermogram substantially as shown in FIG. 14.

In some embodiments, the Formula I, sodium salt, Form II is characterized by a DSC thermogram having one or both of (i) an endothermic transition at about 32° C. and (ii) an endothermic transition at about 122° C. In some embodiments, the Formula I, sodium salt, Form I is characterized by a DSC thermogram having (i) an endothermic transition at about 32° C. and (ii) an endothermic transition at about 122° C.

In some embodiments, the Formula I, sodium salt, Form II is characterized by a TGA curve substantially as shown in FIG. 15.

In some embodiments, the Formula I, sodium salt, Form II loses mass starting at about ambient temperature.

### Compound of Formula I, Sodium Salt, Form III

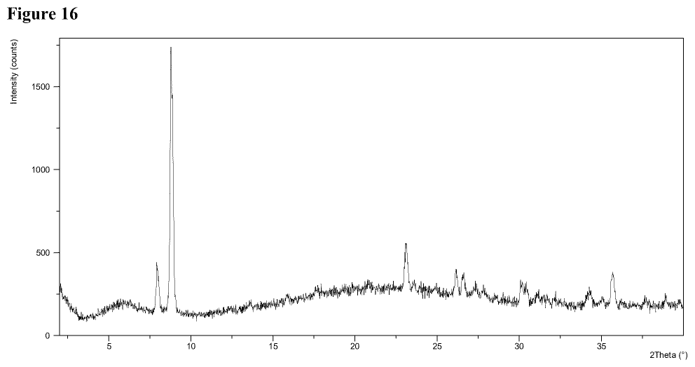

In some embodiments, the crystalline sodium salt of the compound of Formula I is a Form III (“compound of Formula I, sodium salt, Form III” or “Formula I, sodium salt, Form III”). In some embodiments, Compound of Formula I, sodium salt, Form III has an XRPD profile substantially as shown in FIG. 16.

In some embodiments, Formula I, sodium salt, Form III has an XRPD pattern displaying at least two, at least three, at least four, at least five, or at least six of the degree 2θ-reflections with the greatest intensity as the XRPD pattern substantially as shown in FIG. 16.

In some embodiments, the Formula I, sodium salt, Form III has an XRPD pattern comprising 2θ-reflections (+/−0.2 degrees 2θ) at 7.9°, 8.8°, and 23.1°. In some embodiments, the Formula I, sodium salt, Form III has an XRPD pattern comprising 2θ-reflections (+/−0.2 degrees 2θ) at 7.9°, 8.8°, and 23.1, and one, two or three of the 2θ-reflections (+/−0.2 degrees 2θ) at 26.2°, 26.6°, and 30.3°. In some embodiments, the Formula I, sodium salt, Form III has an XRPD pattern comprising 2θ-reflections (+/−0.2 degrees 2θ) at 7.9°, 8.8°, and 23.1°, and one or two of the 2θ-reflections (+/−0.2 degrees 2θ) at 26.2°, 26.6°, and 30.3°. In some embodiments, the Formula I, sodium salt, Form III has an XRPD pattern comprising 2θ-reflections (+/−0.2 degrees 2θ) at 7.9°, 8.8°, and 23.1°, and one of the 2θ-reflections (+/−0.2 degrees 2θ) at 26.2°, 26.6°, and 30.3°. In some embodiments, the Formula I, sodium salt, Form III has an XRPD pattern comprising 2θ-reflections (+/−0.2 degrees 2θ) at 7.9°, 8.8°, and 23.1°, and two of the 20-reflections (+/−0.2 degrees 2θ) at 26.2°, 26.6°, and 30.3°. In some embodiments, the Formula I, sodium salt, Form III has an XRPD pattern comprising 2θ-reflections (+/−0.2 degrees 2θ) at 7.9°, 8.8°, 23.1°, 26.2°, 26.6°, and 30.3°. In some embodiments, the Formula I, sodium salt, Form I has an XRPD pattern comprising any three of the 2θ-reflections (+/−0.2 degrees 2θ) at 7.9°, 8.8°, 23.1°, 26.2°, 26.6°, and 30.3°.

In some embodiments, the Formula I, sodium salt, Form III has an XRPD pattern comprising at least three of the 2θ-reflections (+/−0.2 degrees 2θ) at 7.9°, 8.8°, 23.1°, 26.2°, 26.6°, and 30.3°. In some embodiments, the Formula I, sodium salt, Form III has an XRPD pattern comprising at least four of the 2θ-reflections (+/−0.2 degrees 2θ) at 7.9°, 8.8°, 23.1°, 26.2°, 26.6°, and 30.3°. In some embodiments, the Formula I, sodium salt, Form III has an XRPD pattern comprising at least five of the 2θ-reflections (+/−0.2 degrees 2θ) at 7.9° 8.8°, 23.1°, 26.2°, 26.6°, and 30.3°.

In some embodiments, the Formula I, sodium salt, Form III has an XRPD pattern comprising peaks at:

| Pos. | Rel. Int. |
|---|---|
| [°2Th.] | [%] |
| 7.9 | 18 |
| 8.8 | 100 |
| 23.1 | 17 |
| 26.2 | 9 |
| 26.6 | 7 |
| 30.3 | 5 |

### Compound of Formula I, Sodium Salt, Form IV

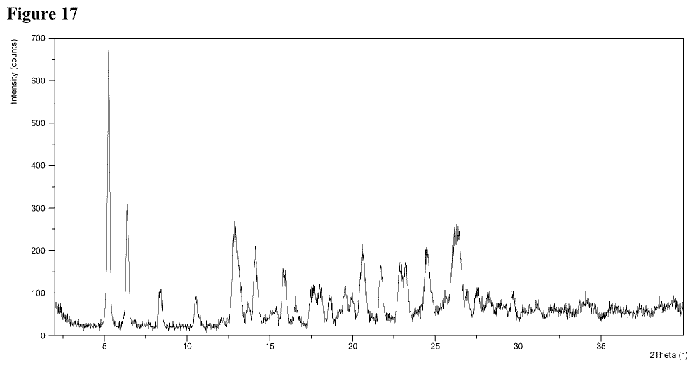

In some embodiments, the crystalline sodium salt of the compound of Formula I is a Form IV (“compound of Formula I, sodium salt, Form IV” or “Formula I, sodium salt, Form IV”). In some embodiments, the compound of Formula I, sodium salt, Form IV has an XRPD profile substantially as shown in FIG. 17.

In some embodiments, Formula I, sodium salt, Form IV has an XRPD pattern displaying at least two, at least three, at least four, at least five, or at least six of the degree 2θ-reflections with the greatest intensity as the XRPD pattern substantially as shown in FIG. 17.

In some embodiments, the Formula I, sodium salt, Form IV has an XRPD pattern comprising 2θ-reflections (+/−0.2 degrees 2θ) at 5.3°, 6.4°, and 12.8°. In some embodiments, the Formula I, sodium salt, Form IV has an XRPD pattern comprising 2θ-reflections (+/−0.2 degrees 2θ) at 5.3°, 6.4°, and 12.8°, and one, two or three of the 2θ-reflections (+/−0.2 degrees 2θ) at 14.1°, 24.5°, and 26.4°. In some embodiments, the Formula I, sodium salt, Form IV has an XRPD pattern comprising 2θ-reflections (+/−0.2 degrees 2θ) at 5.3°, 6.4°, and 12.8°, and one or two of the 2θ-reflections (+/−0.2 degrees 2θ) at 14.1°, 24.5°, and 26.4°. In some embodiments, the Formula I, sodium salt, Form IV has an XRPD pattern comprising 2θ-reflections (+/−0.2 degrees 2θ) at 5.3°, 6.4°, and 12.8°, and one of the 2θ-reflections (+/−0.2 degrees 2θ) at 14.10, 24.5°, and 26.4°. In some embodiments, the Formula I, sodium salt, Form IV has an XRPD pattern comprising 2θ-reflections (+/−0.2 degrees 2θ) at 5.3°, 6.4°, and 12.8°, and two of the 20-reflections (+/−0.2 degrees 2θ) at 14.1°, 24.5°, and 26.4°. In some embodiments, the Formula I, sodium salt, Form IV has an XRPD pattern comprising 2θ-reflections (+/−0.2 degrees 2θ) at 5.3°, 6.4°, 12.8°, 14.1°, 24.5°, and 26.4°. In some embodiments, the Formula I, sodium salt, Form IV has an XRPD pattern comprising any three of the 2θ-reflections (+/−0.2 degrees 2θ) at 5.3°, 6.4°, 12.8°, 14.1°, 24.5°, and 26.4°.

In some embodiments, the Formula I, sodium salt, Form IV has an XRPD pattern comprising 2θ-reflections (+/−0.2 degrees 2θ) at 5.3°, 6.4°, 12.8°, 14.1°, 24.5°, and 26.4°, and one, two, or three of the 2θ-reflections (+/−0.2 degrees 2θ) at 8.4°, 15.8°, and 20.7°. In some embodiments, the Formula I, sodium salt, Form IV has an XRPD pattern comprising 2θ-reflections (+/−0.2 degrees 2θ) at 5.3°, 6.4°, 12.8°, 14.1°, 24.5°, and 26.4°, and one or two of the 2θ-reflections (+/−0.2 degrees 2θ) at 8.4°, 15.8°, and 20.7°. In some embodiments, Formula I, sodium salt, Form IV has an XRPD pattern comprising 2θ-reflections (+/−0.2 degrees 2θ) at 5.3°, 6.4°, 12.8°, 14.1°, 24.5°, and 26.4°, and one of the 2θ-reflections (+/−0.2 degrees 2θ) at 8.4°, 15.8°, and 20.7°. In some embodiments, Formula I, sodium salt, Form IV has an XRPD pattern comprising 2θ-reflections (+/−0.2 degrees 2θ) at 5.3°, 6.4°, 12.8°, 14.1°, 24.5°, and 26.4°, and two of the 2θ-reflections (+/−0.2 degrees 2θ) at 8.4°, 15.8°, and 20.7°. In some embodiments, the Formula I, sodium salt, Form IV has an XRPD pattern comprising 2θ-reflections (+/−0.2 degrees 2θ) at 5.3°, 6.4°, 8.4°, 12.8°, 14.10, 15.8°, 20.7°, 24.5°, and 26.4°. In some embodiments, the Formula I, sodium salt, Form IV has an XRPD pattern comprising any three of the 2θ-reflections (+/−0.2 degrees 2θ) at 5.3°, 6.4°, 8.4°, 12.8°, 14.10, 15.8°, 20.7°, 24.5°, and 26.4°.

In some embodiments, the Formula I, sodium salt, Form IV has an XRPD pattern comprising at least three of the 2θ-reflections (+/−0.2 degrees 2θ) at 5.3°, 6.4°, 8.4°, 12.8°, 14.1°, 15.8°, 20.7°, 24.5°, and 26.4°. In some embodiments, the Formula I, sodium salt, Form IV has an XRPD pattern comprising at least four of the 2θ-reflections (+/−0.2 degrees 2θ) at 5.3°, 6.4°, 8.4°, 12.8°, 14.10, 15.8°, 20.7°, 24.5°, and 26.4°. In some embodiments, the Formula I, sodium salt, Form IV has an XRPD pattern comprising at least five of the 2θ-reflections (+/−0.2 degrees 2θ) at 5.3°, 6.4°, 8.4°, 12.8°, 14.10, 15.8°, 20.7°, 24.5°, and 26.4°.

In some embodiments, the Formula I, sodium salt, Form IV has an XRPD pattern comprising at least three of the 2θ-reflections (+/−0.2 degrees 2θ) 5.3°, 6.4°, 8.4°, 12.8°, 14.10, 15.8°, 20.7°, 21.7°, 22.8°, 23.2°, 24.5°, and 26.4°. In some embodiments, the Formula I, sodium salt, Form IV has an XRPD pattern comprising at least four of the 2θ-reflections (+/−0.2 degrees 2θ) at 5.3° 6.4°, 8.4°, 12.8°, 14.1°, 15.8°, 20.7°, 21.7°, 22.8°, 23.2°, 24.5°, and 26.4°. In some embodiments, the Formula I, sodium salt, Form IV has an XRPD pattern comprising at least five of the 2θ-reflections (+/−0.2 degrees 2θ) at 5.3°, 6.4°, 8.4°, 12.8°, 14.10, 15.8°, 20.7°, 21.7°, 22.8°, 23.2°, 24.5°, and 26.4°.

In some embodiments, the Formula I, sodium salt, Form IV has an XRPD pattern comprising peaks at:

| Pos. | Rel. Int. |
|---|---|
| [°2Th.] | [%] |
| 5.3 | 100 |
| 6.4 | 41 |
| 8.4 | 13 |
| 10.5 | 11 |
| 12.8 | 30 |
| 14.1 | 24 |
| 15.8 | 18 |
| 17.5 | 11 |
| 18.6 | 8 |
| 19.4 | 6 |
| 20.7 | 21 |
| 21.7 | 17 |
| 22.8 | 17 |
| 23.2 | 17 |
| 24.5 | 23 |
| 26.4 | 27 |
| 27.5 | 8 |
| 29.7 | 7 |

### Compound of Formula I, Potassium Salt

In some embodiments, the compound of Formula I can be isolated as a potassium salt which can be amorphous or crystalline. In some embodiments, the potassium salt of the compound of Formula I is crystalline.

### Compound of Formula I, Potassium Salt, Form I

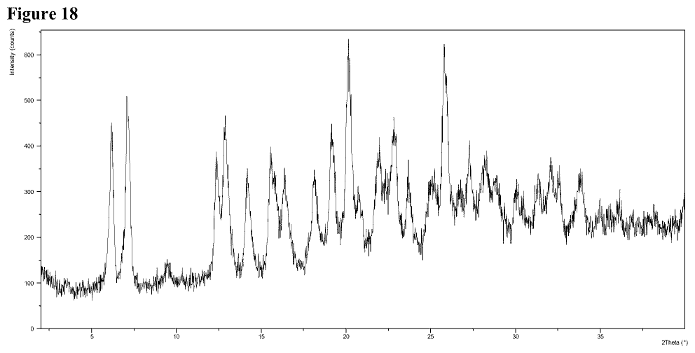

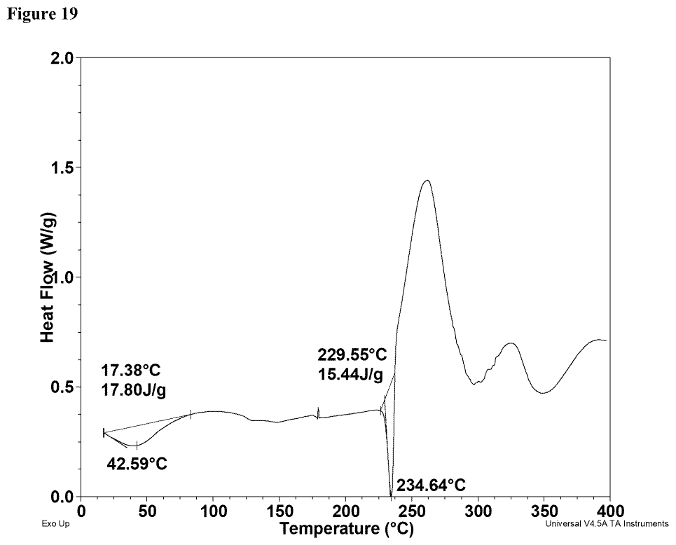

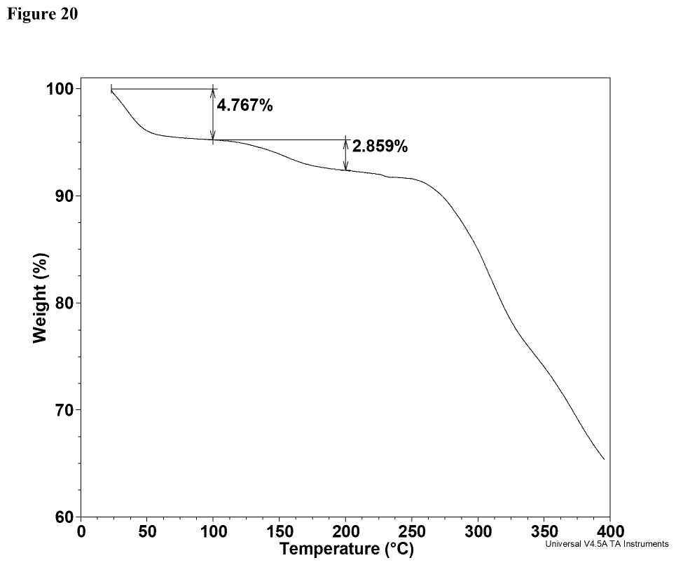

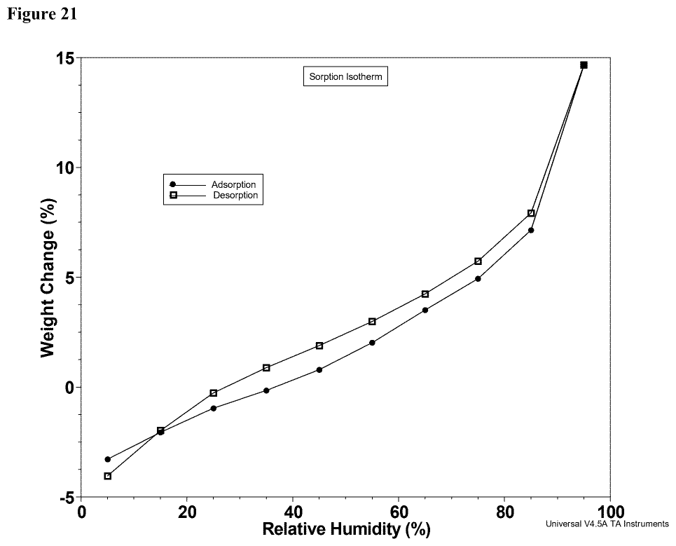

In some embodiments, the crystalline potassium salt of the compound of Formula I is the Form I (“compound of Formula I, potassium salt, Form I” or “Formula I, potassium salt, Form I”). In some embodiments, Formula I, potassium salt, Form I has an XRPD profile substantially as shown in FIG. 18. Formula I, potassium salt, Form I may exhibit a DSC thermogram substantially as shown in FIG. 19. Formula I, potassium salt, Form I may exhibit a TGA graph substantially as shown in FIG. 20. Formula I, potassium salt, Form I may exhibit a DVS curve substantially as shown in FIG. 21.

In some embodiments of Formula I, potassium salt, Form I, at least one, at least two, at least three, or all of the following (a)-(d) apply: (a) Compound of Formula I, potassium salt, Form I has an XRPD pattern substantially as shown in FIG. 18; (b) Compound of Formula I, potassium salt, Form I has a DSC thermogram substantially as shown in FIG. 19; (c) Compound of Formula I, potassium salt, Form I has a TGA curve substantially as shown in FIG. 20; (d) Compound of Formula I, potassium salt, Form I has a DVS curve substantially as shown in FIG. 21.

In some embodiments, Formula I, potassium salt, Form I has an XRPD pattern displaying at least two, at least three, at least four, at least five, or at least six of the degree 2θ-reflections with the greatest intensity as the XRPD pattern substantially as shown in FIG. 18.

In some embodiments, Formula I, potassium salt, Form I has an XRPD pattern comprising 2θ-reflections (+/−0.2 degrees 2θ) at 7.1°, 20.1°, and 25.8°. In some embodiments, Formula I, potassium salt, Form I has an XRPD pattern comprising 2θ-reflections (+/−0.2 degrees 2θ) at 7.1°, 20.1°, and 25.8°, and one, two or three of the 2θ-reflections (+/−0.2 degrees 2θ) at 6.2°, 13.0°, and 19.2°. In some embodiments, Formula I, potassium salt, Form I has an XRPD pattern comprising 2θ-reflections (+/−0.2 degrees 2θ) at 7.1°, 20.1°, and 25.8°, and one or two of the 2θ-reflections (+/−0.2 degrees 2θ) at 6.2°, 13.0°, and 19.2°. In some embodiments, Formula I, potassium salt, Form I has an XRPD pattern comprising 2θ-reflections (+/−0.2 degrees 2θ) at 7.1°, 20.1°, and 25.8°, and one of the 2θ-reflections (+/−0.2 degrees 2θ) at 6.2°, 13.0°, and 19.2°. In some embodiments, Formula I, potassium salt, Form I has an XRPD pattern comprising 2θ-reflections (+/−0.2 degrees 2θ) at 7.1°, 20.1°, and 25.8°, and two of the 20-reflections (+/−0.2 degrees 2θ) at 6.2°, 13.0°, and 19.2°. In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°. In some embodiments, Formula I, potassium salt, Form I has an XRPD pattern comprising any three of the 2θ-reflections (+/−0.2 degrees 2θ) at 6.2°, 7.1°, 13.0°, 19.2°, 20.1°, and 25.8°.

In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°, and one, two or three of the 2θ-reflections (+/−0.2 degrees 2θ) at 14.2°, 15.5°, and 22.9°. In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°, and one or two of the 20-reflections (+/−0.2 degrees 2θ) at 14.2°, 15.5°, and 22.9°. In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°, and one of the 2θ-reflections (+/−0.2 degrees 2θ) at 14.2°, 15.5°, and 22.9°. In some embodiments, Formula I, potassium salt, Form I has an XRPD pattern comprising 2θ-reflections (+/−0.2 degrees 2θ) at 6.2°, 7.1°, 13.0°, 19.2°, 20.1°, and 25.8°, and two of the 2θ-reflections (+/−0.2 degrees 2θ) at 14.2°, 15.5°, and 22.9°. In some embodiments, Formula I, potassium salt, Form I has an XRPD pattern comprising 2θ-reflections (+/−0.2 degrees 2θ) at 6.2°, 7.1°, 13.0°, 14.2°, 15.5°, 19.2°, 20.1°, 22.9°, and 25.8°. In some embodiments, Formula I, potassium salt, Form I has an XRPD pattern comprising any three of the 2θ-reflections (+/−0.2 degrees 2θ) at 6.2°, 7.1°, 13.0°, 14.2°, 15.5°, 19.2°, 20.1°, 22.9°, and 25.8°.

In some embodiments, Formula I, potassium salt, Form I has an XRPD pattern comprising at least three of the 2θ-reflections (+/−0.2 degrees 2θ) at 6.2°, 7.1°, 13.0°, 14.2°, 15.5°, 16.4°, 18.10, 19.2°, 20.1°, 21.9°, 22.9°, and 25.8°. In some embodiments, Formula I, potassium salt, Form I has an XRPD pattern comprising at least four of the 2θ-reflections (+/−0.2 degrees 2θ) at 6.2°, 7.1°, 13.0°, 14.2° 15.5°, 16.4°, 18.1°, 19.2°, 20.1°, 21.9°, 22.9°, and 25.8°. In some embodiments, Formula I, potassium salt, Form I has an XRPD pattern comprising at least five of the 2θ-reflections (+/−0.2 degrees 2θ) at 6.2°, 7.1°, 13.0°, 14.2°, 15.5°, 16.4°, 18.10, 19.2°, 20.1°, 21.9°, 22.9°, and 25.8°. 101.471 In some embodiments, Formula I, potassium salt, Form I has an XRPD pattern comprising peaks at:

| Pos. | Rel. Int. |
|---|---|
| [°2Th.] | [%] |
| 6.2 | 85 |
| 7.1 | 100 |
| 13.0 | 60 |
| 14.2 | 47 |
| 15.5 | 57 |
| 16.4 | 45 |
| 18.1 | 45 |
| 19.2 | 63 |
| 20.1 | 98 |
| 21.9 | 45 |
| 22.9 | 55 |
| 23.7 | 28 |
| 25.8 | 90 |
| 28.6 | 21 |
| 32.1 | 29 |
| 33.8 | 25 |

In some embodiments, Formula I, potassium salt, Form I is characterized by a DSC thermogram substantially as shown in FIG. 19.

In some embodiments, Formula I, potassium salt, Form I is characterized by a DSC thermogram having one or both of (i) an endothermic transition at about 17° C. and (ii) an endothermic transition at about 230° C. In some embodiments, Formula I, potassium salt, Form I is characterized by a DSC thermogram having (i) an endothermic transition at about 17° C. and (ii) comprises an endothermic transition at about 230° C.

In some embodiments, Formula I, potassium salt, Form I is characterized by a TGA curve substantially as shown in FIG. 20. In some embodiments, Formula I, potassium salt, Form I loses mass in multiple stages with one stage starting at about ambient temperature and the next stage starting at about 100° C.

In some embodiments, Formula I, potassium salt, Form I is characterized by a DVS curve substantially as shown in shown in FIG. 21. In some embodiments, Formula I, potassium salt, Form I absorbs more than about 18% of water up to 95% RH at 25° C.

In some embodiments, Formula I, potassium salt, Form I has the following properties:
(a) an XRPD pattern substantially as shown in FIG. 18;
(b) a DSC thermogram substantially as shown in FIG. 19;
(c) a TGA curve substantially as shown in FIG. 20; and
(d) a DVS curve substantially as shown in FIG. 21.

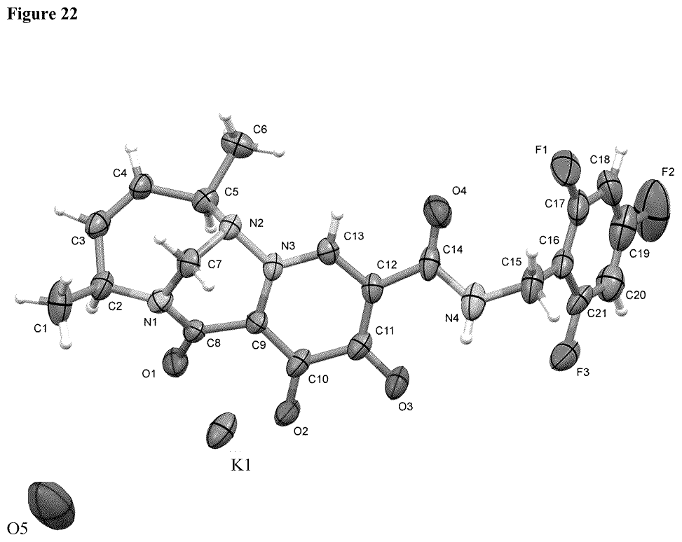

The single crystal data collected on Formula I, potassium salt, Form I are summarized in Table 2 below and also shown in FIG. 22. The crystal system is trigonal and the space group is P3221. The cell parameters and calculated volume are: a=28.6496(4) Å, b=28.6496(4) Å, c=6.89340(10) Å, α=90°, β=90°, γ=120°, V=4900.06(15) Å³. The molecular weight is 494.49 g mol⁻¹ with Z=6, resulting in a calculated density of 1.005 g cm⁻³.

TABLE 2
Crystal Data and Data Collection Parameters
for Formula I, Potassium Salt, Form I

| | |
|---|---|
| Empirical formula | C21H18F3KN4O4.50 |
| Formula weight (g mol−1) | 494.49 |
| Temperature (K) | 150(2) |
| Wavelength (Å) | 1.54184 |
| Crystal system | trigonal |
| Space group | P3₂21 |
| Unit cell parameters | |
| | a = 28.6496(4) Å α = 90° |
| | b = 28.6496(4) Å β = 90° |
| | c = 6.89340(10) Å γ = 120° |
| Unit cell volume (Å3) | 4900.06(15) |
| Cell formula units, Z | 6 |
| Calculated density (g cm−3) | 1.005 |
| Absorption coefficient (mm−1) | 1.819 |
| F(000) | 1524 |
| Crystal size (mm3) | 0.37 × 0.1 × 0.02 |
| Reflections used for cell measurement | 16195 |
| θ range for cell measurement | 3.5560°-75.3450° |
| Total reflections collected | 33594 |
| Index ranges | −35 ≤ h ≤ 33 ; −34 ≤ k ≤ 24; −8≤ l ≤ 8 |
| θ range for data collection | θmin = 3.563°, θmax = 76.309° |
| Completeness to θmax | 98.7% |
| Completeness to θfull = 67.684° | 99.9% |
| Absorption correction | multi-scan |
| Transmission coefficient range | 0.692-1.000 |
| Refinement method | full matrix least-squares on F2 |
| Independent reflections | 6746 [Rint = 0.0379, Rσ = 0.0262] |
| Reflections [ I > 2σ(I) ] | 6485 |
| Reflections / restraints / parameters | 6746 / 0 / 305 |
| Goodness-of-fit on F2 | S = 1.06 |
| Final residuals [ I > 2σ(I) ] | R = 0.0672, Rw = 0.1972 |
| Final residuals [ all reflections ] | R = 0.0691, Rw = 0.1998 |
| Largest diff. peak and hole (e Å−3) | 0.359, −0.324 |
| Max/mean shift/standard uncertainty | 0.000 / 0.000 |
| Absolute structure determination | Flack parameter: 0.0167(7) |
| | Hooft parameter: 0.103(5) |
| | Friedel coverage: 97.2% |

### Compound of Formula I, Potassium Salt, Form II

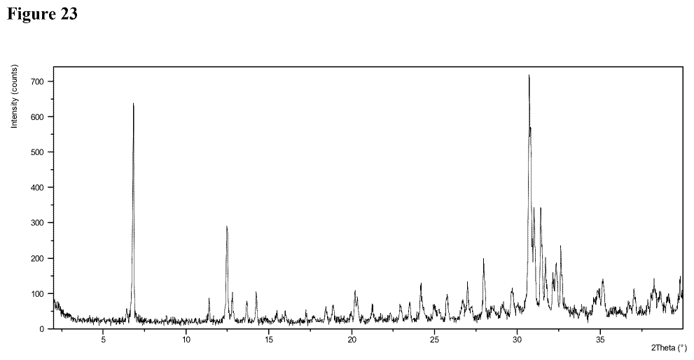

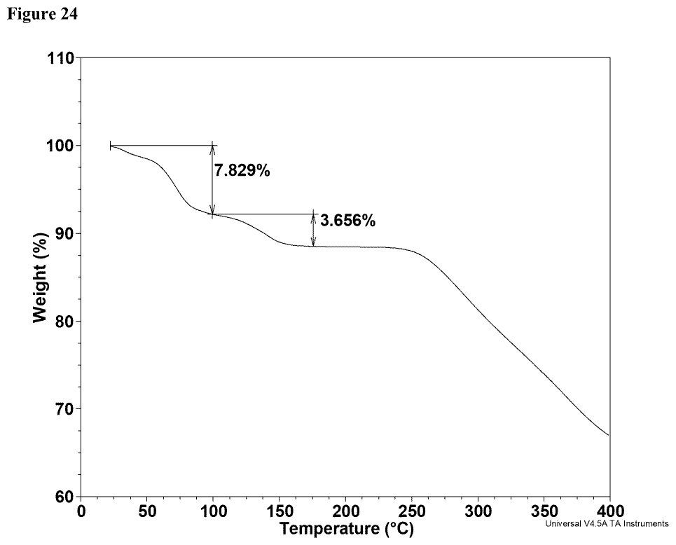

In some embodiments, the crystalline potassium salt of the compound of Formula I is the Form II (“compound of Formula I, potassium salt, Form II” or “Formula I, potassium salt, Form II”). In some embodiments, Formula I, potassium salt, Form II has an XRPD profile substantially as shown in FIG. 23. Compound of Formula I, potassium salt, Form II may exhibit a TGA curve substantially as shown in FIG. 24.

In some embodiments of Compound of Formula I, potassium salt, Form II, one or both of (a) and (b) apply: (a) Compound of Formula I, potassium salt, Form II has an XRPD pattern substantially as shown in FIG. 23; (b) Compound of Formula I, potassium salt, Form II has a TGA curve substantially as shown in FIG. 24.

In some embodiments, Formula I, potassium salt, Form II has an XRPD pattern displaying at least two, at least three, at least four, at least five, or at least six of the degree 20-reflections with the greatest intensity as the XRPD pattern substantially as shown in FIG. 23.

In some embodiments, Formula I, potassium salt, Form II has an XRPD pattern comprising 2θ-reflections (+/−0.2 degrees 2θ) at 6.8°, 30.7°, and 31.4°. In some embodiments, Formula I, potassium salt, Form II has an XRPD pattern comprising 2θ-reflections (+/−0.2 degrees 2θ) at 6.8°, 30.7°, and 31.4°, and one, two or three of the 2θ-reflections (+/−0.2 degrees 2θ) at 12.5°, 28.0°, and 32.6°. In some embodiments, Formula I, potassium salt, Form II has an XRPD pattern comprising 2θ-reflections (+/−0.2 degrees 2θ) at 6.8°, 30.7°, and 31.4°, and one or two of the 2θ-reflections (+/−0.2 degrees 2θ) at 12.5°, 28.0°, and 32.6°. In some embodiments, Formula I, potassium salt, Form II has an XRPD pattern comprising 2θ-reflections (+/−0.2 degrees 2θ) at 6.8°, 30.7°, and 31.4°, and one of the degree 2θ-reflections (+/−0.2 degrees 2θ) at 12.5°, 28.0°, and 32.6°. In some embodiments, Formula I, potassium salt, Form II has an XRPD pattern comprising degree 2θ-reflections (+/−0.2 degrees 2θ) at 6.8°, 30.7°, and 31.4°, and two of the 2θ-reflections (+/−0.2 degrees 2θ) at 12.5°, 28.0°, and 32.6°. In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°. In some embodiments, Formula I, potassium salt, Form II has an XRPD pattern comprising any three of the 2θ-reflections (+/−0.2 degrees 2θ) at 6.8°, 12.5°, 28.0°, 30.7°, 31.4°, and 32.6°.

In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°, and one, two or three of the 2θ-reflections (+/−0.2 degrees 2θ) at 14.3°, 24.2°, and 25.7°. In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°, and one or two of the 20-reflections (+/−0.2 degrees 2θ) at 14.3°, 24.2°, and 25.7°. In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°, and one of the 2θ-reflections (+/−0.2 degrees 2θ) at 14.3°, 24.2°, and 25.7°. In some embodiments, Formula I, potassium salt, Form II has an XRPD pattern comprising 2θ-reflections (+/−0.2 degrees 2θ) at 6.8°, 12.5°, 28.0°, 30.7°, 31.4°, and 32.6°, and two of the 2θ-reflections (+/−0.2 degrees 2θ) at 14.3°, 24.2°, and 25.7°. In some embodiments, Formula I, potassium salt, Form II has an XRPD pattern comprising 2θ-reflections (+/−0.2 degrees 2θ) at 6.8°, 12.5°, 14.3°, 24.2°, 25.7°, 28.0°, 30.7°, 31.4°, and 32.6°. In some embodiments, Formula I, potassium salt, Form II has an XRPD pattern comprising any three of the 2θ-reflections (+/−0.2 degrees 2θ) at 6.8°, 12.5°, 14.3°, 24.2°, 25.7°, 28.0°, 30.7°, 31.4°, and 32.60.

In some embodiments, Formula I, potassium salt, Form II has an XRPD pattern comprising at least three of the 2θ-reflections (+/−0.2 degrees 2θ) at 6.8°, 12.5°, 14.3°, 24.2°, 25.7°, 28.0°, 29.7°, 30.7°, 31.4°, 32.3°, and 32.6°. In some embodiments, Formula I, potassium salt, Form II has an XRPD pattern comprising at least four of the 2θ-reflections (+/−0.2 degrees 2θ) at 6.8°, 12.5°, 14.3°, 24.2°, 25.7°, 28.0°, 29.7°, 30.7°, 31.4°, 32.3°, and 32.6°. In some embodiments, Formula I, potassium salt, Form II has an XRPD pattern comprising at least five of the 2θ-reflections (+/−0.2 degrees 2θ) at 6.8°, 12.5°, 14.3°, 24.2°, 25.7°, 28.0°, 29.7°, 30.7°, 31.4°, 32.3°, and 32.6°.

In some embodiments, Formula I, potassium salt, Form II has an XRPD pattern comprising peaks at:

| Pos. | Rel. Int. |
|---|---|
| [°2Th.] | [%] |
| 6.8 | 94 |
| 12.5 | 41 |
| 14.3 | 11 |
| 20.3 | 8 |
| 24.2 | 14 |
| 25.7 | 10 |
| 26.9 | 6 |
| 28.0 | 21 |
| 29.7 | 10 |
| 30.7 | 100 |
| 31.4 | 43 |
| 32.3 | 12 |
| 32.6 | 23 |
| 35.0 | 9 |

In some embodiments, Formula I, potassium salt, Form II is characterized by a TGA curve substantially as shown in FIG. 24. In some embodiments, Formula I, potassium salt, Form II loses mass in multiple stages with one stage starting at about ambient temperature and the next stage starting at about 100° C.

In some embodiments, Formula I, potassium salt, Form II has the following properties:
(a) an XRPD pattern substantially as shown in FIG. 23; and
(b) a TGA curve substantially as shown in FIG. 24.

### Compound of Formula I, Potassium Salt, Form III

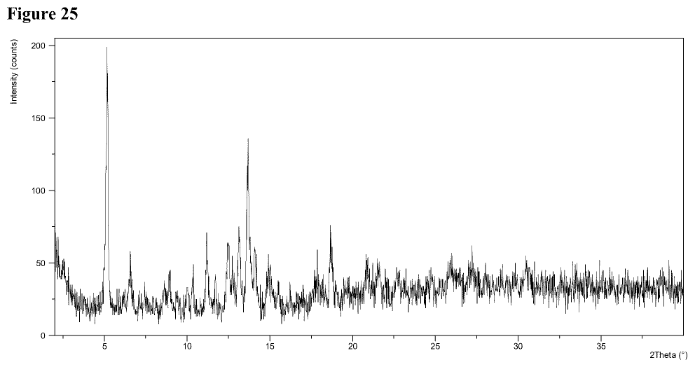

In some embodiments, the crystalline potassium salt of the compound of Formula I is the Form III (“compound of Formula I, potassium salt, Form III” or “Formula I, potassium salt, Form III”). In some embodiments, Formula I, potassium salt, Form III has an XRPD profile substantially as shown in FIG. 25.

In some embodiments, Formula I, potassium salt, Form III has an XRPD pattern displaying at least two, at least three, at least four, at least five, or at least six of the degree 20-reflections with the greatest intensity as the XRPD pattern substantially as shown in FIG. 25.

In some embodiments, Formula I, potassium salt, Form III has an XRPD pattern comprising 2θ-reflections (+/−0.2 degrees 2θ) at 5.2°, 13.2°, and 13.7°. In some embodiments, Formula I, potassium salt, Form III has an XRPD pattern comprising 2θ-reflections (+/−0.2 degrees 2θ) at 5.2°, 13.2°, and 13.7°, and one, two, or three of the 2θ-reflections (+/−0.2 degrees 2θ) at 11.2°, 12.4°, and 18.7°. In some embodiments, Formula I, potassium salt, Form III has an XRPD pattern comprising 2θ-reflections (+/−0.2 degrees 2θ) at 5.2°, 13.2°, and 13.7°, and one or two of the 2θ-reflections (+/−0.2 degrees 2θ) at 11.2°, 12.4°, and 18.7°. In some embodiments, Formula I, potassium salt, Form III has an XRPD pattern comprising 2θ-reflections (+/−0.2 degrees 2θ) at 5.2°, 13.2°, and 13.7°, and one of the degree 2θ-reflections (+/−0.2 degrees 2θ) at 11.2°, 12.4°, and 18.7°. In some embodiments, Formula I, potassium salt, Form III has an XRPD pattern comprising degree 2θ-reflections (+/−0.2 degrees 2θ) at 5.2°, 13.2°, and 13.7°, and two of the 2θ-reflections (+/−0.2 degrees 2θ) at 11.2°, 12.4°, and 18.7°. In some embodiments, Formula I, potassium salt, Form III has an XRPD pattern comprising 2θ-reflections (+/−0.2 degrees 2θ) at 5.2°, 11.2°, 12.4°, 13.2°, 13.7°, and 18.7°. In some embodiments, Formula I, potassium salt, Form III has an XRPD pattern comprising any three of the 2θ-reflections (+/−0.2 degrees 2θ) at 5.2°, 11.2°, 12.4°, 13.2°, 13.7°, and 18.7°.

In some embodiments, Formula I, potassium salt, Form III has an XRPD pattern comprising 2θ-reflections (+/−0.2 degrees 2θ) at 5.2°, 11.2°, 12.4°, 13.2°, 13.7°, and 18.7°, and one or two of the 2θ-reflections (+/−0.2 degrees 2θ) at 6.5° and 15.0°. In some embodiments, Formula I, potassium salt, Form III has an XRPD pattern comprising 2θ-reflections (+/−0.2 degrees 2θ) at 5.2°, 11.2°, 12.4°, 13.2°, 13.7°, and 18.7°, and one of the 2θ-reflections (+/−0.2 degrees 2θ) at 6.5° and 15.0°. In some embodiments, Formula I, potassium salt, Form III has an XRPD pattern comprising 2θ-reflections (+/−0.2 degrees 2θ) at 5.2°, 6.5°, 11.2°, 12.4°, 13.2°, 13.7°, 15.0°, and 18.7°. In some embodiments, Formula I, potassium salt, Form III has an XRPD pattern comprising any three of the 2θ-reflections (+/−0.2 degrees 2θ) at 5.2°, 6.5°, 11.2°, 12.4°, 13.2°, 13.7°, 15.0°, and 18.7°.

In some embodiments, Formula I, potassium salt, Form III has an XRPD pattern comprising at least three of the 2θ-reflections (+/−0.2 degrees 2θ) at 5.2°, 6.5°, 11.2°, 12.4°, 13.2°, 13.7°, 15.0°, and 18.7°. In some embodiments, Formula I, potassium salt, Form III has an XRPD pattern comprising at least four of the 2θ-reflections (+/−0.2 degrees 2θ) at 5.2°, 6.5°, 11.2°, 12.4°, 13.2°, 13.7°, 15.0°, and 18.7°. In some embodiments, Formula I, potassium salt, Form III has an XRPD pattern comprising at least five of the 2θ-reflections (+/−0.2 degrees 2θ) at 5.2°, 6.5°, 11.2°, 12.4°, 13.2°, 13.7°, 15.0°, and 18.7°.

In some embodiments, Formula I, potassium salt, Form III has an XRPD pattern comprising peaks at:

| Pos. | Rel. Int. |
|---|---|
| [°2Th.] | [%] |
| 5.2 | 100 |
| 6.5 | 13 |
| 11.2 | 27 |
| 12.4 | 25 |
| 13.2 | 31 |
| 13.7 | 58 |
| 15.0 | 14 |
| 18.7 | 22 |

### Compound of Formula I, Diethylamine Salt

In some embodiments, the compound of Formula I can be isolated as a diethylamine salt which can be amorphous or crystalline. In some embodiments, the diethylamine salt of the compound of Formula I is crystalline.

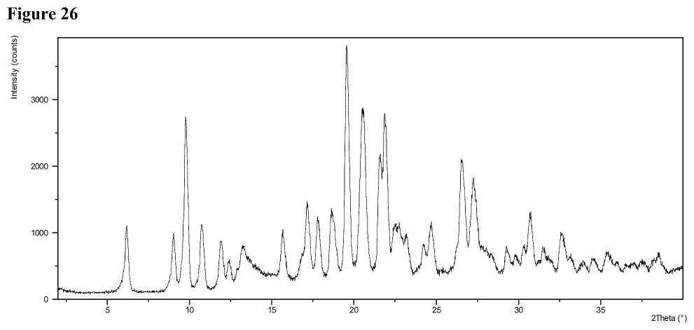

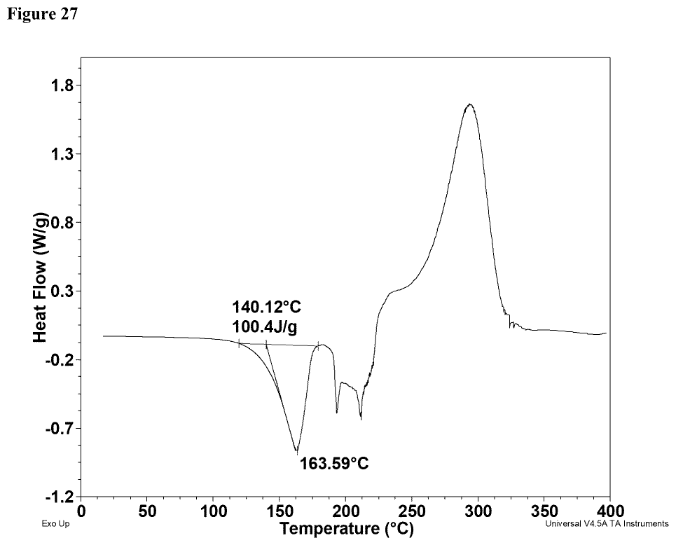

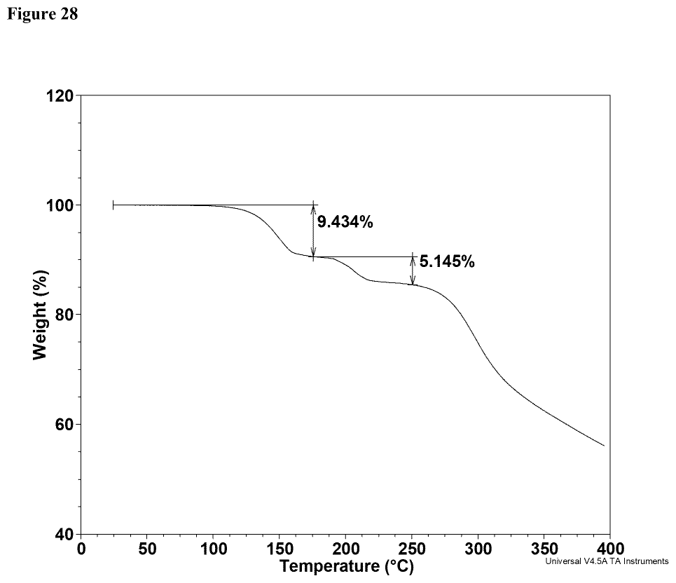

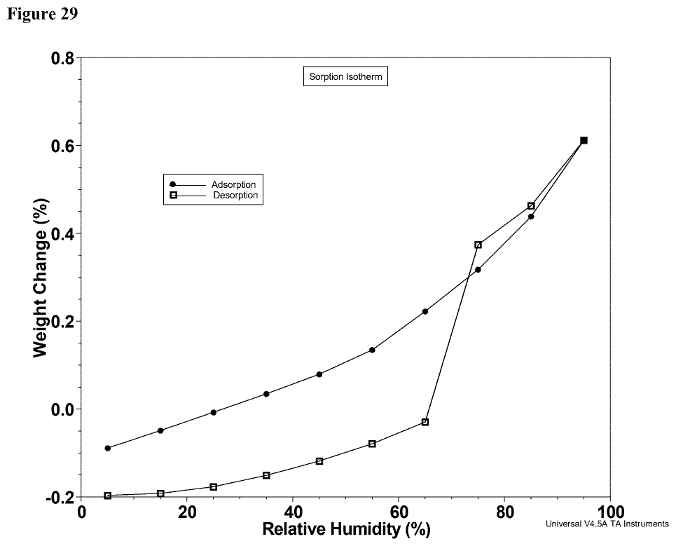

In some embodiments, the crystalline diethylamine salt of the compound of Formula I has an XRPD profile substantially as shown in FIG. 26. The crystalline diethylamine salt of the compound of Formula I may exhibit a DSC thermogram substantially as shown in FIG. 27. The crystalline diethylamine salt of the compound of Formula I may exhibit a TGA graph substantially as shown in FIG. 28. The crystalline diethylamine salt of the compound of Formula I may exhibit a DVS curve substantially as shown in FIG. 29.

In some embodiments of the crystalline diethylamine salt of the compound of Formula I, at least one, at least two, at least three, or all of the following (a)-(d) apply: (a) the crystalline diethylamine salt of the compound of Formula I has an XRPD profile substantially as shown in FIG. 26; (b) the crystalline diethylamine salt of the compound of Formula I exhibits a DSC thermogram substantially as shown in FIG. 27; (c) the crystalline diethylamine salt of the compound of Formula I exhibits a TGA graph substantially as shown in FIG. 28; (d) the crystalline diethylamine salt of the compound of Formula I exhibits a DVS curve substantially as shown in FIG. 29.

In some embodiments, the crystalline diethylamine salt of the compound of Formula I has an XRPD pattern displaying at least two, at least three, at least four, at least five, or at least six of the degree 2θ-reflections with the greatest intensity as the XRPD pattern substantially as shown in FIG. 26.

In some embodiments, the crystalline diethylamine salt of the compound of Formula I has an XRPD pattern comprising 2θ-reflections (+/−0.2 degrees 2θ) at 9.7°, 19.5° and 20.5°. In some embodiments, the crystalline diethylamine salt of the compound of Formula I has an XRPD pattern comprising 2θ-reflections (+/−0.2 degrees 2θ) at 9.7°, 19.5° and 20.5°, and one, two or three of the 2θ-reflections (+/−0.2 degrees 2θ) at 21.9°, 26.5°, and 27.2°. In some embodiments, the crystalline diethylamine salt of the compound of Formula I has an XRPD pattern comprising 2θ-reflections (+/−0.2 degrees 2θ) at 9.7°, 19.5° and 20.5°, and one or two of the 2θ-reflections (+/−0.2 degrees 2θ) at 21.9°, 26.5°, and 27.2°. In some embodiments, the crystalline diethylamine salt of the compound of Formula I has an XRPD pattern comprising 2θ-reflections (+/−0.2 degrees 2θ) at 9.7°, 19.5° and 20.5°, and one of the 2θ-reflections (+/−0.2 degrees 2θ) at 21.9°, 26.5°, and 27.2°. In some embodiments, the crystalline diethylamine salt of the compound of Formula I has an XRPD pattern comprising 2θ-reflections (+/−0.2 degrees 2θ) at 9.7°, 19.5° and 20.5°, and two of the 2θ-reflections (+/−0.2 degrees 2θ) at 21.9°, 26.5°, and 27.2°. In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°. In some embodiments, the crystalline diethylamine salt of the compound of Formula I has an XRPD pattern comprising any three of the 2θ-reflections (+/−0.2 degrees 2θ) at 9.7°, 19.5°, 20.5°, 21.9°, 26.5°, and 27.2°.

In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°, and one, two or three of the 2θ-reflections (+/−0.2 degrees 2θ) at 6.2°, 10.7°, and 17.1°. In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°, and one or two of the 2θ-reflections (+/−0.2 degrees 2θ) at 6.2°, 10.7°, and 17.1°. In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°, and one of the degree 2θ-reflections (+/−0.2 degrees 2θ) at 6.2°, 10.7°, and 17.1°. In some embodiments, the crystalline diethylamine salt of the compound of Formula I has an XRPD pattern comprising 2θ-reflections (+/−0.2 degrees 2θ) at 9.7°, 19.5°, 20.5°, 21.9°, 26.5°, and 27.2°, and two of the 2θ-reflections (+/−0.2 degrees 2θ) at 6.2°, 10.7°, and 17.1°. In some embodiments, the crystalline diethylamine salt of the compound of Formula I has an XRPD pattern comprising 2θ-reflections (+/−0.2 degrees 2θ) at 6.2°, 9.7°, 10.7°, 17.1°, 19.5°, 20.5°, 21.9°, 26.5°, and 27.2°. In some embodiments, the crystalline diethylamine salt of the compound of Formula I has an XRPD pattern comprising any three of the 2θ-reflections (+/−0.2 degrees 2θ) at 6.2°, 9.7°, 10.7°, 17.1°, 19.5°, 20.5°, 21.9°, 26.5°, and 27.2°.

In some embodiments, the crystalline diethylamine salt of the compound of Formula I has an XRPD pattern comprising at least three of the 2θ-reflections (+/−0.2 degrees 2θ) at 6.2°, 9.0°, 9.7°, 10.7°, 17.1°, 17.8°, 18.6°, 19.5°, 20.5°, 21.5°, 21.9°, 26.5°, 27.2°, and 30.7°. In some embodiments, the crystalline diethylamine salt of the compound of Formula I has an XRPD pattern comprising at least four of the 2θ-reflections (+/−0.2 degrees 2θ) at 6.2°, 9.0°, 9.7°, 10.7°, 17.1°, 17.8°, 18.6°, 19.5°, 20.5°, 21.5°, 21.9°, 26.5°, 27.2°, and 30.7°. In some embodiments, the crystalline diethylamine salt of the compound of Formula I has an XRPD pattern comprising at least five of the 2θ-reflections (+/−0.2 degrees 2θ) at 6.2°, 9.0°, 9.7°, 10.7°, 17.1°, 17.8°, 18.6°, 19.5°, 20.5°, 21.5°, 21.9°, 26.5°, 27.2°, and 30.7°.

In some embodiments, the crystalline diethylamine salt of the compound of Formula I has an XRPD pattern comprising peaks at:

| Pos. | Rel. Int. |
|---|---|
| [°2Th.] | [%] |
| 6.2 | 29 |
| 9.0 | 24 |
| 9.7 | 72 |
| 10.7 | 28 |
| 11.9 | 20 |
| 12.4 | 9 |
| 13.2 | 13 |
| 15.7 | 19 |
| 17.1 | 32 |
| 17.8 | 25 |
| 18.6 | 26 |
| 19.5 | 100 |
| 20.5 | 69 |
| 21.5 | 49 |
| 21.9 | 68 |
| 23.2 | 15 |
| 24.7 | 21 |
| 26.5 | 48 |
| 27.2 | 38 |
| 30.7 | 23 |
| 32.5 | 13 |
| 35.4 | 7 |

In some embodiments, the crystalline diethylamine salt of the compound of Formula I is characterized by a DSC thermogram substantially as shown in FIG. 27. In some embodiments, the crystalline diethylamine salt of the compound of Formula I is characterized by a DSC thermogram having an endothermic transition at about 140° C.

In some embodiments, the crystalline diethylamine salt of the compound of Formula I is characterized by a TGA curve substantially as shown in FIG. 28. In some embodiments, the crystalline diethylamine salt of the compound of Formula I loses mass in multiple stages with one stage starting at about 100° C. and the next stage starting at about 175° C.

In some embodiments, the crystalline diethylamine salt of the compound of Formula I is characterized by a DVS curve substantially as shown in shown in FIG. 29. In some embodiments, the crystalline diethylamine salt of the compound of Formula I absorbs less than about 1% of water up to 95% RH at 25° C.

In some embodiments, the crystalline diethylamine salt of the compound of Formula I has the following properties:
(a) an XRPD pattern substantially as shown in FIG. 26;
(b) a DSC thermogram substantially as shown in FIG. 27;
(c) a TGA curve substantially as shown in FIG. 28; and
(d) a DVS curve substantially as shown in FIG. 29.

### Compound of Formula I, Ammonia Salt

In some embodiments, the compound of Formula I can be isolated as an ammonia salt which can be amorphous or crystalline. In some embodiments, the ammonia salt of the compound of Formula I is crystalline.

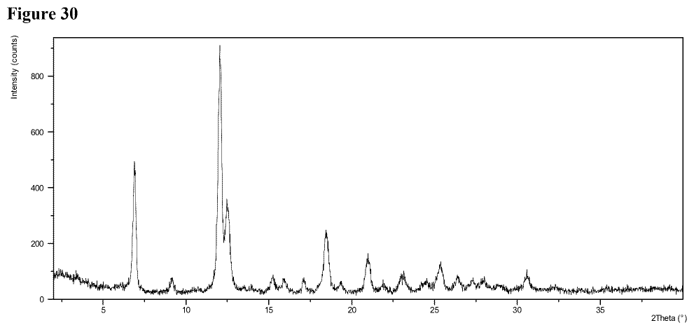

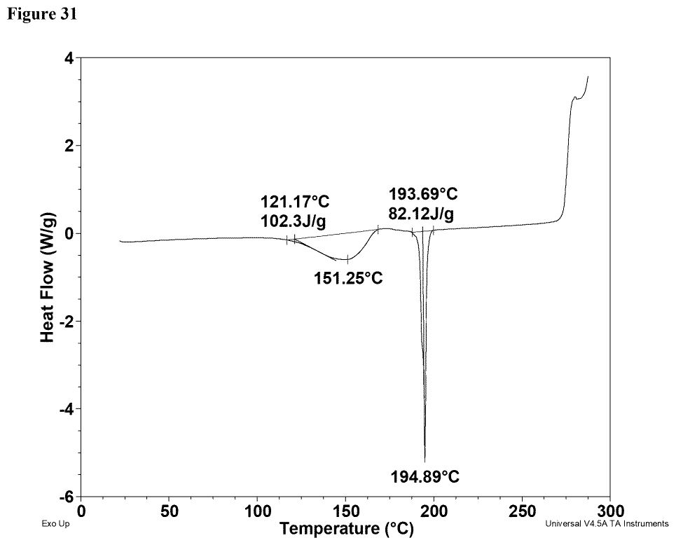

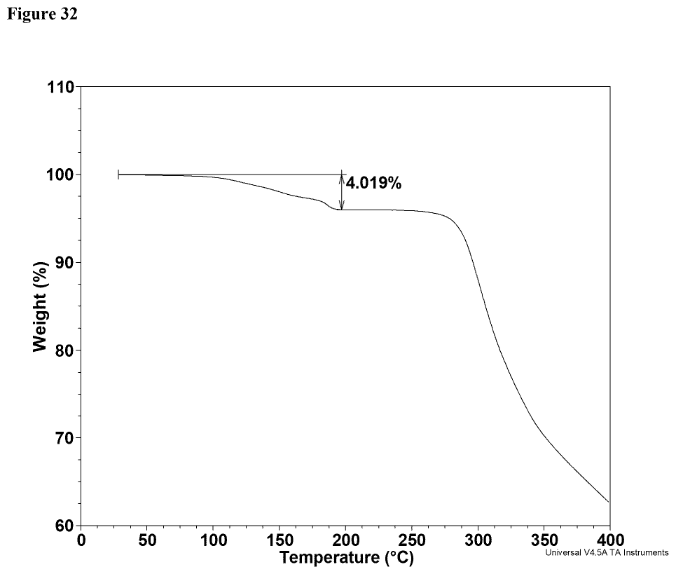

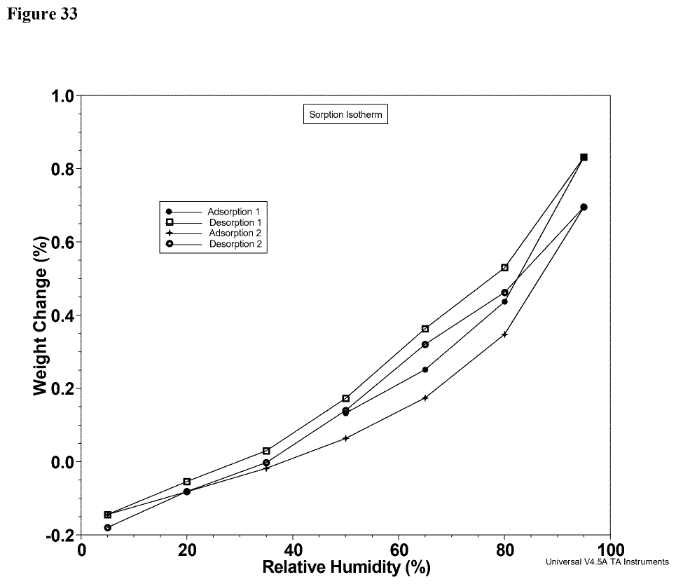

In some embodiments, the crystalline ammonia salt of the compound of Formula I has an XRPD profile substantially as shown in FIG. 30. The crystalline ammonia salt of the compound of Formula I may exhibit a DSC thermogram substantially as shown in FIG. 31. The crystalline ammonia salt of the compound of Formula I may exhibit a TGA graph substantially as shown in FIG. 32. The crystalline ammonia salt of the compound of Formula I may exhibit a DVS curve substantially as shown in FIG. 33.

In some embodiments of the crystalline ammonia salt of the compound of Formula I, at least one, at least two, at least three, or all of the following (a)-(d) apply: (a) the crystalline ammonia salt of the compound of Formula I has an XRPD profile substantially as shown in FIG. 30; (b) the crystalline ammonia salt of the compound of Formula I exhibits a DSC thermogram substantially as shown in FIG. 31; (c) the crystalline ammonia salt of the compound of Formula I exhibits a TGA graph substantially as shown in FIG. 32; (d) the crystalline ammonia salt of the compound of Formula I exhibits a DVS curve substantially as shown in FIG. 33.

In some embodiments, the crystalline ammonia salt of the compound of Formula I has an XRPD pattern displaying at least two, at least three, at least four, at least five, or at least six of the degree 2θ-reflections with the greatest intensity as the XRPD pattern substantially as shown in FIG. 30.

In some embodiments, the crystalline ammonia salt of the compound of Formula I has an XRPD pattern comprising 2θ-reflections (+/−0.2 degrees 2θ) at 6.9°, 12.1°, and 12.5°. In some embodiments, the crystalline ammonia salt of the compound of Formula I has an XRPD pattern comprising 2θ-reflections (+/−0.2 degrees 2θ) at 6.9°, 12.1°, and 12.5°, and one, two or three of the 2θ-reflections (+/−0.2 degrees 2θ) at 18.4°, 21.0°, and 25.4°. In some embodiments, the crystalline ammonia salt of the compound of Formula I has an XRPD pattern comprising 2θ-reflections (+/−0.2 degrees 2θ) at 6.9°, 12.1°, and 12.5°, and one or two of the 2θ-reflections (+/−0.2 degrees 2θ) at 18.4°, 21.0°, and 25.4°. In some embodiments, the crystalline ammonia salt of the compound of Formula I has an XRPD pattern comprising 2θ-reflections (+/−0.2 degrees 2θ) at 6.9°, 12.1°, and 12.5°, and one of the 2θ-reflections (+/−0.2 degrees 2θ) at 18.4°, 21.0°, and 25.4°. In some embodiments, the crystalline ammonia salt of the compound of Formula I has an XRPD pattern comprising 2θ-reflections (+/−0.2 degrees 2θ) at 6.9°, 12.1°, and 12.5°, and two of the 2θ-reflections (+/−0.2 degrees 2θ) at 18.4°, 21.0°, and 25.4°. In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°. In some embodiments, the crystalline ammonia salt of the compound of Formula I has an XRPD pattern comprising any three of the 2θ-reflections (+/−0.2 degrees 2θ) at 6.9°, 12.1°, 12.5°, 18.4°, 21.0°, and 25.4°.

In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°, and one, two or three of the 2θ-reflections (+/−0.2 degrees 2θ) at 9.2°, 23.1°, and 30.6°. In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°, and one or two of the 2θ-reflections (+/−0.2 degrees 2θ) at 9.2°, 23.1°, and 30.6°. In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°, and one of the 2θ-reflections (+/−0.2 degrees 2θ) at 9.2°, 23.1°, and 30.6°. In some embodiments, the crystalline ammonia salt of the compound of Formula I has an XRPD pattern comprising 2θ-reflections (+/−0.2 degrees 2θ) at 6.9°, 12.1°, 12.5°, 18.4°, 21.0°, and 25.4°, and two of the 2θ-reflections (+/−0.2 degrees 2θ) at 9.2°, 23.1°, and 30.6°. In some embodiments, the crystalline ammonia salt of the compound of Formula I has an XRPD pattern comprising 2θ-reflections (+/−0.2 degrees 2θ) at 6.9°, 9.2°, 12.1°, 12.5°, 18.4°, 21.0°, 23.1°, 25.4°, and 30.6°. In some embodiments, the crystalline ammonia salt of the compound of Formula I has an XRPD pattern comprising any three of the 2θ-reflections (+/−0.2 degrees 2θ) at 6.9°, 9.2°, 12.1°, 12.5°, 18.4°, 21.0°, 23.1°, 25.4°, and 30.6°.

In some embodiments, the crystalline ammonia salt of the compound of Formula I has an XRPD pattern comprising at least three of the 2θ-reflections (+/−0.2 degrees 2θ) at 6.9°, 9.2°, 12.1°, 12.5°, 15.6°, 17.1°, 18.4°, 21.0°, 23.1°, 25.4°, 26.4°, and 30.6°. In some embodiments, the crystalline ammonia salt of the compound of Formula I has an XRPD pattern comprising at least four of the 2θ-reflections (+/−0.2 degrees 2θ) at 6.9°, 9.2°, 12.1°, 12.5°, 15.6°, 17.1°, 18.4°, 21.0°, 23.1°, 25.4°, 26.4°, and 30.6°. In some embodiments, the crystalline ammonia salt of the compound of Formula I has an XRPD pattern comprising at least five of the 2θ-reflections (+/−0.2 degrees 2θ) at 6.9°, 9.2° 12.1°, 12.5°, 15.6°, 17.1°, 18.4°, 21.0°, 23.1°, 25.4°, 26.4°, and 30.60.

In some embodiments, the crystalline ammonia salt of the compound of Formula I has an XRPD pattern comprising peaks at:

| Pos. | Rel. Int. |
|---|---|
| [°2Th.] | [%] |
| 6.9 | 56 |
| 9.2 | 6 |
| 12.1 | 100 |
| 12.5 | 38 |
| 15.6 | 2 |
| 17.1 | 4 |
| 18.4 | 25 |
| 21.0 | 15 |
| 23.1 | 6 |
| 25.4 | 11 |
| 26.4 | 5 |
| 30.6 | 6 |

In some embodiments, the crystalline ammonia salt of the compound of Formula I is characterized by a DSC thermogram substantially as shown in FIG. 31. In some embodiments, the crystalline ammonia salt of the compound of Formula I is characterized by a DSC thermogram having one or both of (i) endothermic transition at about 121° C. and (ii) an endothermic transition at about 194° C. In some embodiments, the crystalline ammonia salt of the compound of Formula I is characterized by a DSC thermogram having (i) endothermic transition at about 121° C. and (ii) an endothermic transition at about 194° C.

In some embodiments, the crystalline ammonia salt of the compound of Formula I is characterized by a TGA curve substantially as shown in FIG. 32. In some embodiments, the crystalline ammonia salt of the compound of Formula I loses mass starting at about 80° C.

In some embodiments, the crystalline ammonia salt of the compound of Formula I is characterized by a DVS curve substantially as shown in shown in FIG. 33. In some embodiments, the crystalline ammonia salt of the compound of Formula I absorbs less than about 1% of water up to 95% RH at 25° C.

In some embodiments, the crystalline ammonia salt of the compound of Formula I has the following properties:
(a) an XRPD pattern substantially as shown in FIG. 30;
(b) a DSC thermogram substantially as shown in FIG. 31;
(c) a TGA curve substantially as shown in FIG. 32; and
(d) a DVS curve substantially as shown in FIG. 33.

### Compound of Formula I, Calcium Salt

In some embodiments, the compound of Formula I can be isolated as a calcium salt which can be in any form such as, for example, crystalline or amorphous. In some embodiments, the calcium salt of the compound of Formula I is crystalline.

### Compound of Formula I, Calcium Salt, Form I

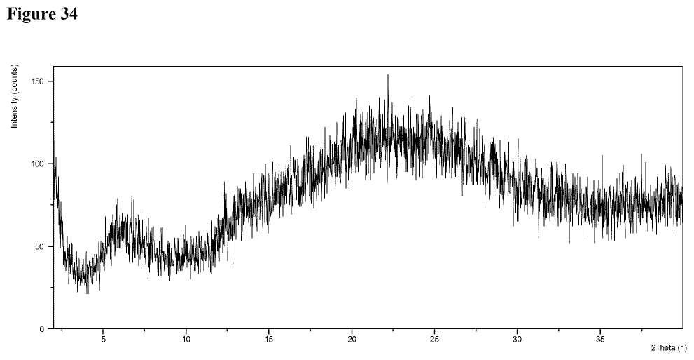

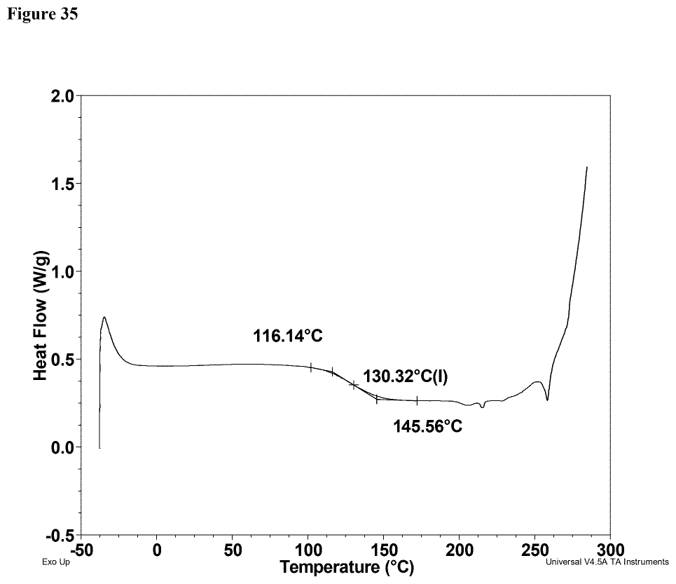

In some embodiments, the crystalline calcium salt of the compound of Formula I is a Form I (“compound of Formula I, calcium salt, Form I” or “Formula I, calcium salt, Form I”) having an XRPD profile substantially as shown in FIG. 34. Compound of Formula I, calcium salt, Form I may exhibit a DSC thermogram substantially as shown in FIG. 35.

In some embodiments of Compound of Formula I, calcium salt, Form I, one or both of the (a) and (b) apply: (a) the Compound of Formula I, calcium salt, Form I has an XRPD profile substantially as shown in FIG. 34; (b) the Compound of Formula I, calcium salt, Form I exhibits a DSC thermogram substantially as shown in FIG. 35.

In some embodiments, the Compound of Formula I, calcium salt, Form I is characterized by a DSC thermogram substantially as shown in FIG. 35. In some embodiments, the Compound of Formula I, calcium salt, Form I is characterized by a DSC thermogram having a glass transition at about 130° C.

In some embodiments, the Compound of Formula I, calcium salt, Form I has the following properties:
(a) an XRPD pattern substantially as shown in FIG. 34; and
(b) a DSC thermogram substantially as shown in FIG. 35.

### Compound of Formula I, Calcium Salt, Form II

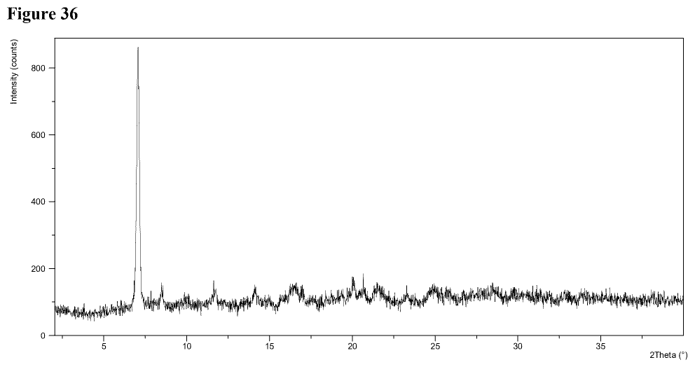

In some embodiments, the crystalline calcium salt of the compound of Formula I is a Form II (“compound of Formula I, calcium salt, Form II” or “Formula I, calcium salt, Form II”). In some embodiments, the compound of Formula I, calcium salt, Form II has an XRPD profile substantially as shown in FIG. 36.

In some embodiments, Formula I, calcium salt, Form II has an XRPD pattern displaying at least two, at least three, at least four, at least five, or at least six of the degree 2θ-reflections with the greatest intensity as the XRPD pattern substantially as shown in FIG. 36.

In some embodiments, the Formula I, calcium salt, Form II has an XRPD pattern comprising 2θ-reflections (+/−0.2 degrees 2θ) at 7.1°, 8.5°, and 11.7°. In some embodiments, the Formula I, calcium salt, Form II has an XRPD pattern comprising 2θ-reflections (+/−0.2 degrees 2θ) at 7.1°, 8.5°, and 11.7°, and one, two or three of the 2θ-reflections (+/−0.2 degrees 2θ) at 14.1°, 16.5°, and 20.1°. In some embodiments, the Formula I, calcium salt, Form II has an XRPD pattern comprising 2θ-reflections (+/−0.2 degrees 2θ) at 7.1°, 8.5°, and 11.7°, and one or two of the 2θ-reflections (+/−0.2 degrees 2θ) at 14.1°, 16.5°, and 20.1°. In some embodiments, the Formula I, calcium salt, Form II has an XRPD pattern comprising 2θ-reflections (+/−0.2 degrees 2θ) at 7.1°, 8.5°, and 11.7°, and one of the 2θ-reflections (+/−0.2 degrees 2θ) at 14.1°, 16.5°, and 20.1°. In some embodiments, the Formula I, calcium salt, Form II has an XRPD pattern comprising 2θ-reflections (+/−0.2 degrees 2θ) at 7.1°, 8.5°, and 11.7°, and two of the 20-reflections (+/−0.2 degrees 2θ) at 14.1°, 16.5°, and 20.1°. In some embodiments, the Formula I, calcium salt, Form II has an XRPD pattern comprising 2θ-reflections (+/−0.2 degrees 2θ) at 7.1°, 8.5°, 11.7°, 14.1°, 16.5°, and 20.1°. In some embodiments, the Formula I, calcium salt, Form II has an XRPD pattern comprising any three of the 2θ-reflections (+/−0.2 degrees 2θ) at 7.1°, 8.5°, 11.7°, 14.1°, 16.5°, and 20.1°.

In some embodiments, the Formula I, calcium salt, Form II has an XRPD pattern comprising at least three of the 2θ-reflections (+/−0.2 degrees 2θ) at 7.1°, 8.5°, 11.7°, 14.10, 16.5°, and 20.1°. In some embodiments, the Formula I, calcium salt, Form II has an XRPD pattern comprising at least four of the 2θ-reflections (+/−0.2 degrees 2θ) at 7.1°, 8.5°, 11.7°, 14.1°, 16.5°, and 20.1°. In some embodiments, the Formula I, calcium salt, Form II has an XRPD pattern comprising at least five of the 2θ-reflections (+/−0.2 degrees 2θ) at 7.1°, 8.5°, 11.7°, 14.1°, 16.5°, and 20.1°.

In some embodiments, the Formula I, calcium salt, Form II has an XRPD pattern comprising peaks at:

| Pos. | Rel. Int. |
|---|---|
| [°2Th.] | [%] |
| 7.1 | 100 |
| 8.5 | 7 |
| 11.7 | 5 |
| 14.1 | 6 |
| 16.5 | 7 |
| 20.1 | 7 |
| 21.5 | 5 |
| 24.8 | 5 |

### Compound of Formula I, Calcium Salt, Form III

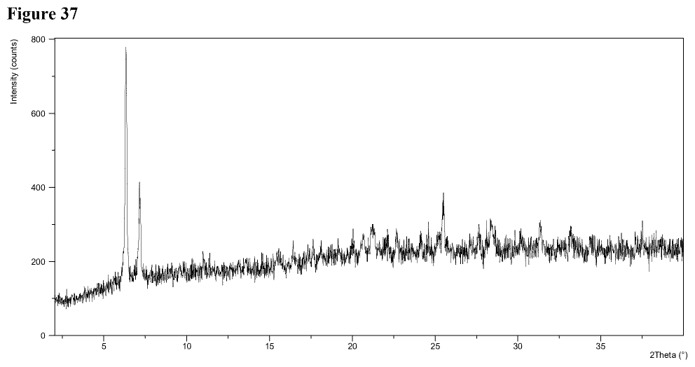

In some embodiments, the crystalline calcium salt of the compound of Formula I is a Form III (“compound of Formula I, calcium salt, Form III” or “Formula I, calcium salt, Form III”). In some embodiments, the compound of Formula I, calcium salt, Form III has an XRPD profile substantially as shown in FIG. 37.

In some embodiments, Formula I, calcium salt, Form III has an XRPD pattern displaying at least two, at least three, at least four, at least five, or at least six of the degree 2θ-reflections with the greatest intensity as the XRPD pattern substantially as shown in FIG. 37.

In some embodiments, the Formula I, calcium salt, Form III has an XRPD pattern comprising 2θ-reflections (+/−0.2 degrees 2θ) at 6.3°, 7.2°, and 25.5°. In some embodiments, the Formula I, calcium salt, Form III has an XRPD pattern comprising 2θ-reflections (+/−0.2 degrees 2θ) at 6.3°, 7.2°, and 25.5°, and one, two or three of the 2θ-reflections (+/−0.2 degrees 2θ) at 21.2°, 28.4°, and 31.4°. In some embodiments, the Formula I, calcium salt, Form III has an XRPD pattern comprising 2θ-reflections (+/−0.2 degrees 2θ) at 6.3°, 7.2°, and 25.5°, and one or two of the 2θ-reflections (+/−0.2 degrees 2θ) at 21.2°, 28.4°, and 31.4°. In some embodiments, the Formula I, calcium salt, Form III has an XRPD pattern comprising 2θ-reflections (+/−0.2 degrees 2θ) at 6.3°, 7.2°, and 25.5°, and one of the 2θ-reflections (+/−0.2 degrees 2θ) at 21.2°, 28.4°, and 31.4°. In some embodiments, the Formula I, calcium salt, Form III has an XRPD pattern comprising 2θ-reflections (+/−0.2 degrees 2θ) at 6.3°, 7.2°, and 25.5°, and two of the 20-reflections (+/−0.2 degrees 2θ) at 21.2°, 28.4°, and 31.4°. In some embodiments, the Formula I, calcium salt, Form III has an XRPD pattern comprising 2θ-reflections (+/−0.2 degrees 2θ) at 6.3°, 7.2°, 21.2°, 25.5°, 28.4°, and 31.4°. In some embodiments, the Formula I, calcium salt, Form III has an XRPD pattern comprising any three of the 2θ-reflections (+/−0.2 degrees 2θ) at 6.3°, 7.2°, 21.2°, 25.5°, 28.4°, and 31.4°.

In some embodiments, the Formula I, calcium salt, Form III has an XRPD pattern comprising at least three of the 2θ-reflections (+/−0.2 degrees 2θ) at 6.3°, 7.2°, 21.2°, 25.5°, 28.4°, and 31.4°. In some embodiments, the Formula I, calcium salt, Form III has an XRPD pattern comprising at least four of the 2θ-reflections (+/−0.2 degrees 2θ) at 6.3°, 7.2°, 21.2°, 25.5°, 28.4°, and 31.4°. In some embodiments, the Formula I, calcium salt, Form III has an XRPD pattern comprising at least five of the 2θ-reflections (+/−0.2 degrees 2θ) at 6.3°, 7.2°, 21.2°, 25.5°, 28.4°, and 31.4°.

In some embodiments, the Formula I, calcium salt, Form III has an XRPD pattern comprising peaks at:

| Pos. | Rel. Int. |
|---|---|
| [°2Th.] | [%] |
| 6.3 | 100 |
| 7.2 | 35 |
| 21.2 | 11 |
| 25.5 | 22 |
| 28.4 | 11 |
| 31.4 | 9 |
| 6.3 | 100 |
| 7.2 | 35 |

### Compound of Formula I, Magnesium Salt

In some embodiments, the compound of Formula I can be isolated as a magnesium salt which can be in any form such as, for example, crystalline or amorphous. In some embodiments, the magnesium salt of the compound of Formula I is crystalline.

### Compound of Formula I, Magnesium Salt, Form I

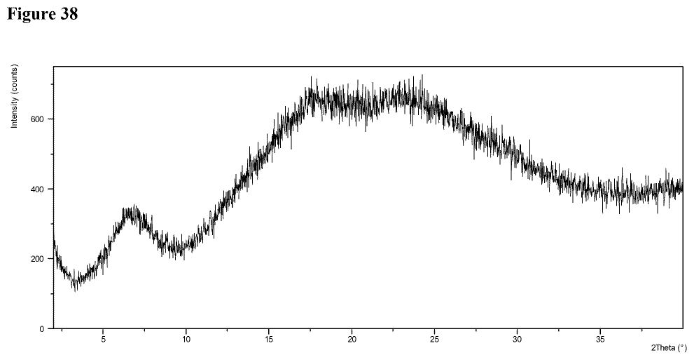

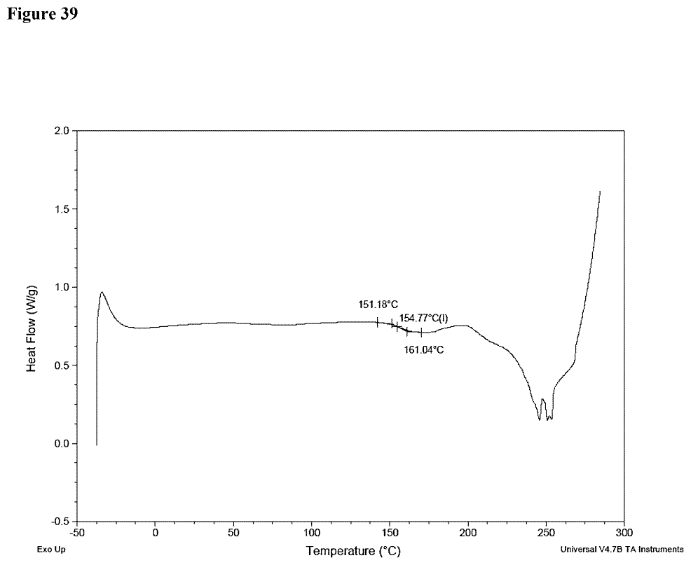

In some embodiments, the crystalline magnesium salt of the compound of Formula I is a Form I (“compound of Formula I, magnesium salt, Form I” or “Formula I, magnesium salt, Form I). In some embodiments, Compound of Formula I, magnesium salt, Form I has an XRPD profile substantially as shown in FIG. 38. The Formula I, magnesium salt, Form I may exhibit a DSC thermogram substantially as shown in FIG. 39.

In some embodiments of Formula I, magnesium salt, Form I, one or both of the (a) and (b) apply: (a) Formula I, magnesium salt, Form I has an XRPD profile substantially as shown in FIG. 38; (b) Formula I, magnesium salt, Form I exhibits a DSC thermogram substantially as shown in FIG. 39.

In some embodiments, Formula I, magnesium salt, Form I is characterized by a DSC thermogram substantially as shown in FIG. 39. In some embodiments, Formula I, magnesium salt, Form I is characterized by a DSC thermogram having a glass transition at about 155° C.

In some embodiments, the Formula I, magnesium salt, Form I has the following properties:
(a) an XRPD pattern substantially as shown in FIG. 38; and
(b) a DSC thermogram substantially as shown in FIG. 39.

### Compound of Formula I, Magnesium Salt, Form II

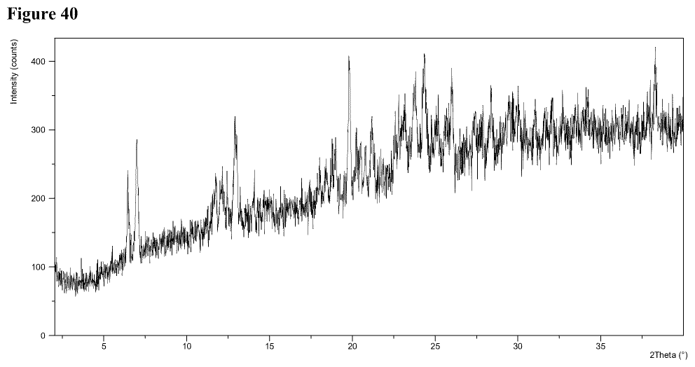

In some embodiments, the crystalline magnesium salt of the compound of Formula I is a Form II (“compound of Formula I, magnesium salt, Form II” or “Formula I, magnesium salt, Form II). In some embodiments, Compound of Formula I, magnesium salt, Form II has an XRPD profile substantially as shown in FIG. 40.

In some embodiments, Formula I, magnesium salt, Form II has an XRPD pattern displaying at least two, at least three, at least four, at least five, or at least six of the degree 20-reflections with the greatest intensity as the XRPD pattern substantially as shown in FIG. 40.

In some embodiments, the Formula I, magnesium salt, Form II has an XRPD pattern comprising 2θ-reflections (+/−0.2 degrees 2θ) at 7.0°, 19.8°, and 24.3°. In some embodiments, the Formula I, magnesium salt, Form II has an XRPD pattern comprising 2θ-reflections (+/−0.2 degrees 2θ) at 7.0°, 19.8°, and 24.3°, and one, two or three of the 2θ-reflections (+/−0.2 degrees 2θ) at 6.5°, 13.0°, and 21.2°. In some embodiments, the Formula I, magnesium salt, Form II has an XRPD pattern comprising 2θ-reflections (+/−0.2 degrees 2θ) at 7.0°, 19.8°, and 24.3°, and one or two of the 2θ-reflections (+/−0.2 degrees 2θ) at 6.5°, 13.0°, and 21.2°. In some embodiments, the Formula I, magnesium salt, Form II has an XRPD pattern comprising 2θ-reflections (+/−0.2 degrees 2θ) at 7.0°, 19.8°, and 24.3°, and one of the 2θ-reflections (+/−0.2 degrees 2θ) at 6.5°, 13.0°, and 21.2°. In some embodiments, the Formula I, magnesium salt, Form II has an XRPD pattern comprising 2θ-reflections (+/−0.2 degrees 2θ) at 7.0°, 19.8°, and 24.3°, and two of the 2θ-reflections (+/−0.2 degrees 2θ) at 6.5°, 13.0°, and 21.2°. In some embodiments, the Formula I, magnesium salt, Form II has an XRPD pattern comprising 2θ-reflections (+/−0.2 degrees 2θ) at 6.5°, 7.0°, 13.0°, 19.8°, 21.2°, and 24.3°. In some embodiments, the Formula I, magnesium salt, Form II has an XRPD pattern comprising any three of the 2θ-reflections (+/−0.2 degrees 2θ) at 6.5°, 7.0°, 13.0°, 19.8°, 21.2°, and 24.3°.

In some embodiments, the Formula I, magnesium salt, Form II has an XRPD pattern comprising at least three of the 2θ-reflections (+/−0.2 degrees 2θ) at 6.5°, 7.0°, 13.0°, 19.8°, 21.2°, and 24.3°. In some embodiments, the Formula I, magnesium salt, Form II has an XRPD pattern comprising at least four of the 2θ-reflections (+/−0.2 degrees 2θ) at 6.5°, 7.0°, 13.0°, 19.8°, 21.2°, and 24.3°. In some embodiments, the Formula I, magnesium salt, Form II has an XRPD pattern comprising at least five of the 2θ-reflections (+/−0.2 degrees 2θ) at 6.5°, 7.0°, 13.0°, 19.8°, 21.2°, and 24.3°.

In some embodiments, the Formula I, magnesium salt, Form II has an XRPD pattern comprising peaks at:

| Pos. | Rel. Int. |
|---|---|
| [°2Th.] | [%] |
| 6.5 | 51 |
| 7.0 | 88 |
| 13.0 | 65 |
| 19.8 | 100 |
| 21.2 | 44 |
| 24.3 | 77 |
| 25.9 | 38 |

### Compound of Formula I, N-butylamine Salt

In some embodiments, the compound of Formula I can be isolated as a N-butylamine salt which can be amorphous or crystalline. In some embodiments, the N-butylamine salt of the compound of Formula I is crystalline.

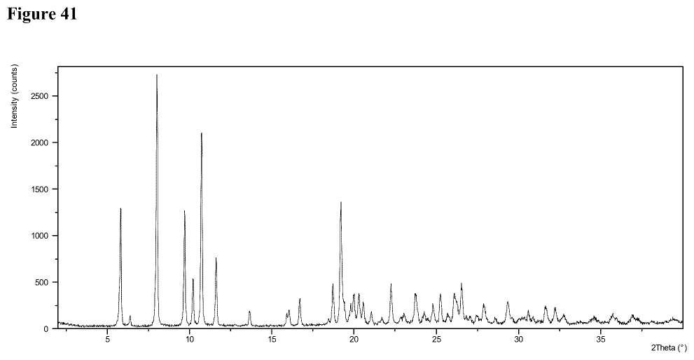

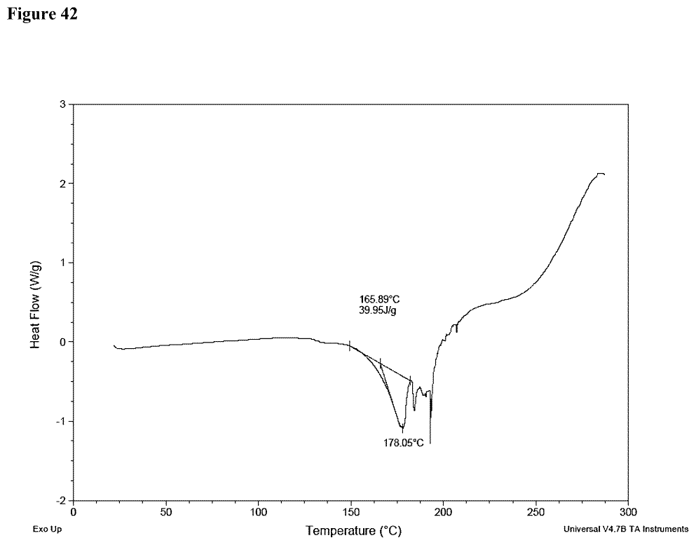

In some embodiments, the crystalline N-butylamine salt of the compound of Formula I has an XRPD profile substantially as shown in FIG. 41. The crystalline N-butylamine salt of the compound of Formula I may exhibit a DSC thermogram substantially as shown in FIG. 42.

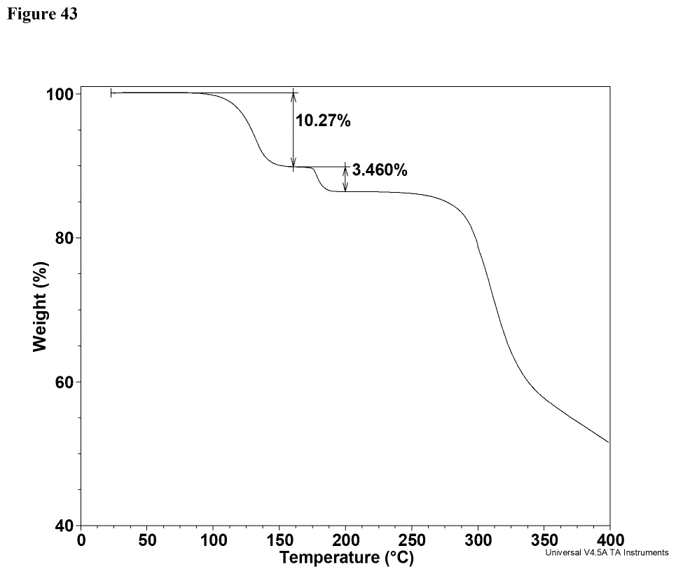

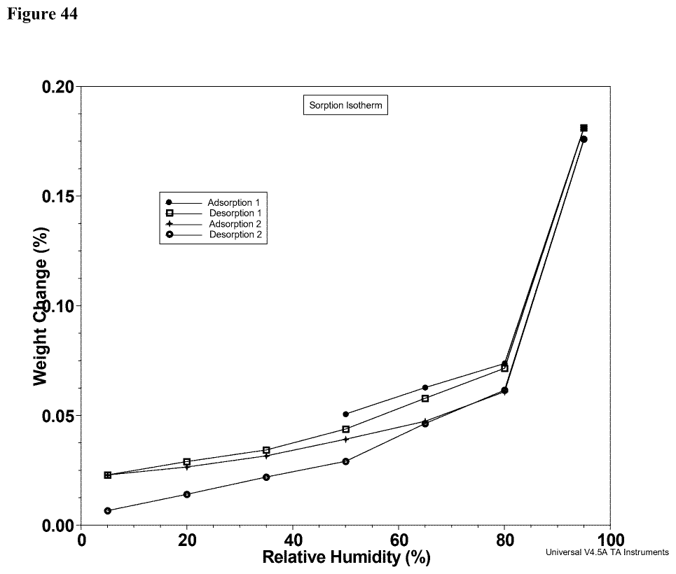

The crystalline N-butylamine salt of the compound of Formula I may exhibit a TGA graph substantially as shown in FIG. 43. The crystalline N-butylamine salt of the compound of Formula I may exhibit a DVS curve substantially as shown in FIG. 44.

In some embodiments of the crystalline N-butylamine salt of the compound of Formula I, at least one, at least two, at least three, or all of the following (a)-(d) apply: (a) the crystalline N-butylamine salt of the compound of Formula I has an XRPD profile substantially as shown in FIG. 41; (b) the crystalline N-butylamine salt of the compound of Formula I exhibits a DSC thermogram substantially as shown in FIG. 42; (c) the crystalline N-butylamine salt of the compound of Formula I exhibits a TGA graph substantially as shown in FIG. 43; (d) the crystalline N-butylamine salt of the compound of Formula I exhibits a DVS curve substantially as shown in FIG. 44.

In some embodiments, the crystalline N-butylamine salt of the compound of Formula I has an XRPD pattern displaying at least two, at least three, at least four, at least five, or at least six of the degree 2θ-reflections with the greatest intensity as the XRPD pattern substantially as shown in FIG. 41.

In some embodiments, the crystalline N-butylamine salt of the compound of Formula I has an XRPD pattern comprising 2θ-reflections (+/−0.2 degrees 2θ) at 5.8°, 8.0°, and 10.7°. In some embodiments, the crystalline N-butylamine salt of the compound of Formula I has an XRPD pattern comprising 2θ-reflections (+/−0.2 degrees 2θ) at 5.8°, 8.0°, and 10.7°, and one, two or three of the 2θ-reflections (+/−0.2 degrees 2θ) at 9.7°, 11.6°, and 19.2°. In some embodiments, the crystalline N-butylamine salt of the compound of Formula I has an XRPD pattern comprising 2θ-reflections (+/−0.2 degrees 2θ) at 5.8°, 8.0°, and 10.7°, and one or two of the 2θ-reflections (+/−0.2 degrees 2θ) at 9.7°, 11.6°, and 19.2°. In some embodiments, the crystalline N-butylamine salt of the compound of Formula I has an XRPD pattern comprising 2θ-reflections (+/−0.2 degrees 2θ) at 5.8°, 8.0°, and 10.7°, and one of the 2θ-reflections (+/−0.2 degrees 2θ) at 9.7°, 11.6°, and 19.2°. In some embodiments, the crystalline N-butylamine salt of the compound of Formula I has an XRPD pattern comprising 2θ-reflections (+/−0.2 degrees 2θ) at 5.8°, 8.0°, and 10.7°, and two of the 2θ-reflections (+/−0.2 degrees 2θ) at 9.7°, 11.6°, and 19.2°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°. In some embodiments, the crystalline N-butylamine salt of the compound of Formula I has an XRPD pattern comprising any three of the 2θ-reflections (+/−0.2 degrees 2θ) at 5.8°, 8.0°, 9.7°, 10.7°, 11.6°, and 19.2°.

In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°, and one, two or three of the 2θ-reflections (+/−0.2 degrees 2θ) at 18.7°, 22.3°, and 26.5°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°, and one or two of the 2θ-reflections (+/−0.2 degrees 2θ) at 18.7°, 22.3°, and 26.5°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°, and one of the 2θ-reflections (+/−0.2 degrees 2θ) at 18.7°, 22.3°, and 26.5°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, and 19.2°, and two of the 2θ-reflections (+/−0.2 degrees 2θ) at 18.7°, 22.3°, and 26.5°. In some embodiments, the crystalline N-butylamine salt of the compound of Formula I has an XRPD pattern comprising 2θ-reflections (+/−0.2 degrees 2θ) at 5.8°, 8.0°, 9.7°, 10.7°, 11.6°, 18.7°, 19.2°, 22.3°, and 26.5°. In some embodiments, the crystalline N-butylamine salt of the compound of Formula I has an XRPD pattern comprising any three of the 2θ-reflections (+/−0.2 degrees 2θ) at 5.8°, 8.0°, 9.7°, 10.7°, 11.6°, 18.7°, 19.2°, 22.3°, and 26.5°.

In some embodiments, the crystalline N-butylamine salt of the compound of Formula I has an XRPD pattern comprising at least three of the 2θ-reflections (+/−0.2 degrees 2θ) at 5.8°, 8.0°, 9.7° 10.2°, 10.7°, 11.6°, 16.7°, 18.7°, 19.2°, 20.3°, 22.3°, 23.7°, 25.3°, 26.1°, and 26.5°. In some embodiments, the crystalline N-butylamine salt of the compound of Formula I has an XRPD pattern comprising at least four of the 2θ-reflections (+/−0.2 degrees 2θ) at 5.8°, 8.0°, 9.7° 10.2°, 10.7°, 11.6°, 16.7°, 18.7°, 19.2°, 20.3°, 22.3°, 23.7°, 25.3°, 26.1°, and 26.5°. In some embodiments, the crystalline N-butylamine salt of the compound of Formula I has an XRPD pattern comprising at least five of the 2θ-reflections (+/−0.2 degrees 2θ) at 5.8°, 8.0°, 9.7°, 10.2°, 10.7°, 11.6°, 16.7°, 18.7°, 19.2°, 20.3°, 22.3°, 23.7°, 25.3°, 26.1°, and 26.5°. In some embodiments, the crystalline N-butylamine salt of the compound of Formula I has an XRPD pattern comprising peaks at:

| Pos. | Rel. Int. |
|---|---|
| [°2Th.] | [%] |
| 5.8 | 48 |
| 8.0 | 100 |
| 9.7 | 45 |
| 10.2 | 17 |
| 10.7 | 78 |
| 11.6 | 26 |
| 16.0 | 5 |
| 16.7 | 10 |
| 18.7 | 16 |
| 19.2 | 45 |
| 20.3 | 11 |
| 22.3 | 13 |
| 23.0 | 3 |
| 23.7 | 11 |
| 25.3 | 10 |
| 26.1 | 10 |
| 26.5 | 14 |
| 27.9 | 7 |
| 31.6 | 6 |

In some embodiments, the crystalline N-butylamine salt of the compound of Formula I is characterized by a DSC thermogram substantially as shown in FIG. 42.

In some embodiments, the crystalline N-butylamine salt of the compound of Formula I is characterized by a DSC thermogram having an endothermic transition with an onset at about 166° C.

In some embodiments, the crystalline N-butylamine salt of the compound of Formula I is characterized by a TGA curve substantially as shown in FIG. 43. In some embodiments, the crystalline N-butylamine salt of the compound of Formula I loses mass in multiples stages starting at about 80° C. and another stage starting at about 175° C.

In some embodiments, the crystalline N-butylamine salt of the compound of Formula I is characterized by a DVS curve substantially as shown in shown in FIG. 44. In some embodiments, the crystalline N-butylamine salt of the compound of Formula I absorbs less than about 0.2% of water up to 95% RH at 25° C.

In some embodiments, the crystalline N-butylamine salt of the compound of Formula I has the following properties:
(a) an XRPD pattern substantially as shown in FIG. 41;
(b) a DSC thermogram substantially as shown in FIG. 42;
(c) a TGA curve substantially as shown in FIG. 43; and
(d) a DVS curve substantially as shown in FIG. 44.

### Compound of Formula I, Diethanolamine Salt

In some embodiments, the compound of Formula I can be isolated as a diethanolamine salt which can be amorphous or crystalline. In some embodiments, the diethanolamine salt of the compound of Formula I is crystalline.

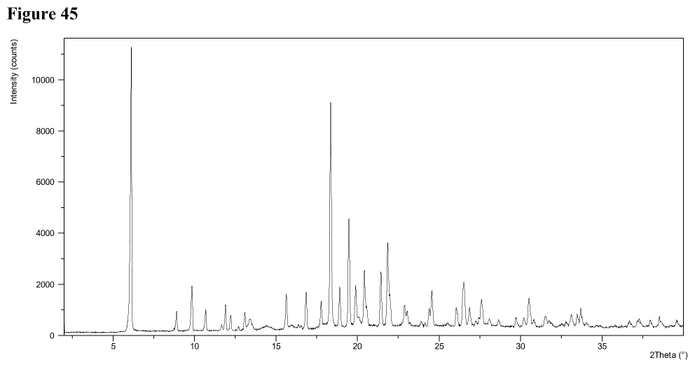

In some embodiments, the crystalline diethanolamine salt of the compound of Formula I has an XRPD profile substantially as shown in FIG. 45. In some embodiments, the crystalline diethanolamine salt of the compound of Formula I has an XRPD pattern displaying at least two, at least three, at least four, at least five, or at least six of the degree 2θ-reflections with the greatest intensity as the XRPD pattern substantially as shown in FIG. 45.

In some embodiments, the crystalline diethanolamine salt of the compound of Formula I has an XRPD pattern comprising 2θ-reflections (+/−0.2 degrees 2θ) at 6.1°, 18.4°, and 19.5°. In some embodiments, the crystalline diethanolamine salt of the compound of Formula I has an XRPD pattern comprising 2θ-reflections (+/−0.2 degrees 2θ) at 6.1°, 18.4°, and 19.5°, and one, two or three of the 2θ-reflections (+/−0.2 degrees 2θ) at 9.9°, 20.4°, and 21.8°. In some embodiments, the crystalline diethanolamine salt of the compound of Formula I has an XRPD pattern comprising 2θ-reflections (+/−0.2 degrees 2θ) at 6.1°, 18.4°, and 19.5°, and one or two of the 2θ-reflections (+/−0.2 degrees 2θ) at 9.9°, 20.4°, and 21.8°. In some embodiments, the crystalline diethanolamine salt of the compound of Formula I has an XRPD pattern comprising 2θ-reflections (+/−0.2 degrees 2θ) at 6.1°, 18.4°, and 19.5°, and one of the 2θ-reflections (+/−0.2 degrees 2θ) at 9.9°, 20.4°, and 21.8°. In some embodiments, the crystalline diethanolamine salt of the compound of Formula I has an XRPD pattern comprising 2θ-reflections (+/−0.2 degrees 2θ) at 6.1°, 18.4°, and 19.5°, and two of the 2θ-reflections (+/−0.2 degrees 2θ) at 9.9°, 20.4°, and 21.8°. In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°. In some embodiments, the crystalline diethanolamine salt of the compound of Formula I has an XRPD pattern comprising any three of the 2θ-reflections (+/−0.2 degrees 2θ) at 6.1°, 9.9°, 18.4°, 19.5°, 20.4°, and 21.8°.

In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°, and one, two or three of the 2θ-reflections (+/−0.2 degrees 2θ) at 16.9°, 18.9°, and 26.5°. In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°, and one or two of the 2θ-reflections (+/−0.2 degrees 2θ) at 16.9°, 18.9°, and 26.5°. In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°, and one of the 2θ-reflections (+/−0.2 degrees 2θ) at 16.9°, 18.9°, and 26.5°. In some embodiments, the crystalline diethanolamine salt of the compound of Formula I has an XRPD pattern comprising 2θ-reflections (+/−0.2 degrees 2θ) at 6.1°, 9.9°, 18.4°, 19.5°, 20.4°, and 21.8°, and two of the 2θ-reflections (+/−0.2 degrees 2θ) at 16.9°, 18.9°, and 26.5°. In some embodiments, the crystalline diethanolamine salt of the compound of Formula I has an XRPD pattern comprising 2θ-reflections (+/−0.2 degrees 2θ) at 6.1°, 9.9°, 16.9°, 18.4°, 18.9°, 19.5°, 20.4°, 21.8°, and 26.5°. In some embodiments, the crystalline diethanolamine salt of the compound of Formula I has an XRPD pattern comprising any three of the 2θ-reflections (+/−0.2 degrees 2θ) at 6.1°, 9.9°, 16.9°, 18.4°, 18.9°, 19.5°, 20.4°, 21.8°, and 26.50.

In some embodiments, the crystalline diethanolamine salt of the compound of Formula I has an XRPD pattern comprising at least three of the 2θ-reflections (+/−0.2 degrees 2θ) at 6.1°, 9.9°, 16.9°, 18.4°, 18.9°, 19.5°, 20.4°, 21.8°, and 26.5°. In some embodiments, the crystalline diethanolamine salt of the compound of Formula I has an XRPD pattern comprising at least four of the 2θ-reflections (+/−0.2 degrees 2θ) at 6.1°, 9.9°, 16.9°, 18.4°, 18.9°, 19.5°, 20.4°, 21.8°, and 26.5°. In some embodiments, the crystalline diethanolamine salt of the compound of Formula I has an XRPD pattern comprising at least five of the 2θ-reflections (+/−0.2 degrees 2θ) at 6.1°, 9.90, 16.90, 18.40, 18.90, 19.50, 20.40, 21.80, and 26.50.

In some embodiments, the crystalline diethanolamine salt of the compound of Formula I has an XRPD pattern comprising peaks at:

| Pos. | Rel. Int. |
|---|---|
| [°2Th.] | [%] |
| 6.1 | 100 |
| 9.9 | 16 |
| 11.9 | 9 |
| 13.2 | 1 |
| 15.6 | 12 |
| 16.9 | 13 |
| 18.4 | 79 |
| 18.9 | 14 |
| 19.5 | 38 |
| 20.4 | 20 |
| 21.4 | 19 |
| 21.8 | 29 |
| 22.9 | 5 |
| 24.6 | 11 |
| 26.5 | 15 |
| 30.5 | 10 |
| 33.5 | 2 |

### Compound of Formula I, Ethylenediamine Salt

In some embodiments, the compound of Formula I can be isolated as an ethylenediamine salt which can be amorphous or crystalline. In some embodiments, the ethylenediamine salt of the compound of Formula I is crystalline.

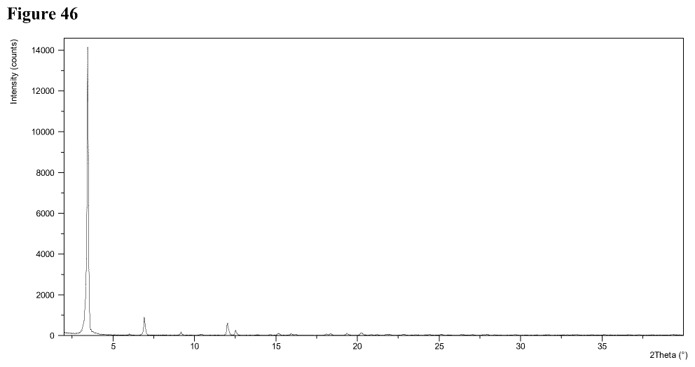

In some embodiments, the crystalline ethylenediamine salt of the compound of Formula I has an XRPD profile substantially as shown in FIG. 46. In some embodiments, the crystalline ethylenediamine salt of the compound of Formula I has an XRPD pattern displaying at least two, at least three, at least four, at least five, or at least six of the degree 2θ-reflections with the greatest intensity as the XRPD pattern substantially as shown in FIG. 46.

In some embodiments, the crystalline ethylenediamine salt of the compound of Formula I has an XRPD pattern comprising 2θ-reflections (+/−0.2 degrees 2θ) at 3.5°, 6.9°, and 12.0°. In some embodiments, the crystalline ethylenediamine salt of the compound of Formula I has an XRPD pattern comprising 2θ-reflections (+/−0.2 degrees 2θ) at 3.5°, 6.9°, and 12.0°, and one, two or three of the 2θ-reflections (+/−0.2 degrees 2θ) at 9.2°, 12.5°, and 20.2°. In some embodiments, the crystalline ethylenediamine salt of the compound of Formula I has an XRPD pattern comprising 2θ-reflections (+/−0.2 degrees 2θ) at 3.5°, 6.9°, and 12.0°, and one or two of the 2θ-reflections (+/−0.2 degrees 2θ) at 9.2°, 12.5°, and 20.2°. In some embodiments, the crystalline ethylenediamine salt of the compound of Formula I has an XRPD pattern comprising 2θ-reflections (+/−0.2 degrees 2θ) at 3.5°, 6.9°, and 12.0°, and one of the 2θ-reflections (+/−0.2 degrees 2θ) at 9.2°, 12.5°, and 20.2°. In some embodiments, the crystalline ethylenediamine salt of the compound of Formula I has an XRPD pattern comprising 2θ-reflections (+/−0.2 degrees 2θ) at 3.5°, 6.9°, and 12.0°, and two of the 2θ-reflections (+/−0.2 degrees 2θ) at 9.2°, 12.5°, and 20.2°. In some embodiments, the crystalline ethylenediamine salt of the compound of Formula I has an XRPD pattern comprising 2θ-reflections (+/−0.2 degrees 2θ) at 3.5°, 6.9°, 9.2°, 12.0°, 12.5°, and 20.2°. In some embodiments, the crystalline ethylenediamine salt of the compound of Formula I has an XRPD pattern comprising any three of the 2θ-reflections (+/−0.2 degrees 2θ) at 3.5°, 6.9°, 9.2°, 12.0°, 12.5°, and 20.2°.

In some embodiments, the crystalline ethylenediamine salt of the compound of Formula I has an XRPD pattern comprising at least three of the 2θ-reflections (+/−0.2 degrees 2θ) at 3.5°, 6.9°, 9.2°, 12.0°, 12.5°, and 20.2°. In some embodiments, the crystalline ethylenediamine salt of the compound of Formula I has an XRPD pattern comprising at least four of the 2θ-reflections (+/−0.2 degrees 2θ) at 3.5°, 6.9°, 9.2°, 12.0°, 12.5°, and 20.2°. In some embodiments, the crystalline ethylenediamine salt of the compound of Formula I has an XRPD pattern comprising at least five of the 2θ-reflections (+/−0.2 degrees 2θ) at 3.5°, 6.9°, 9.2°, 12.0°, 12.5°, and 20.2°.

In some embodiments, the crystalline ethylenediamine salt of the compound of Formula I has an XRPD pattern comprising peaks at:

| Pos. | Rel. Int. |
|---|---|
| [°2Th.] | [%] |
| 3.5 | 100 |
| 6.9 | 6 |
| 9.2 | 1 |
| 12.0 | 4 |
| 12.5 | 1 |
| 15.2 | 1 |
| 19.4 | 1 |
| 20.2 | 1 |

### Compound of Formula I, Morpholine Salt

In some embodiments, the compound of Formula I can be isolated as a morpholine salt which can be amorphous or crystalline. In some embodiments, the morpholine salt of the compound of Formula I is crystalline.

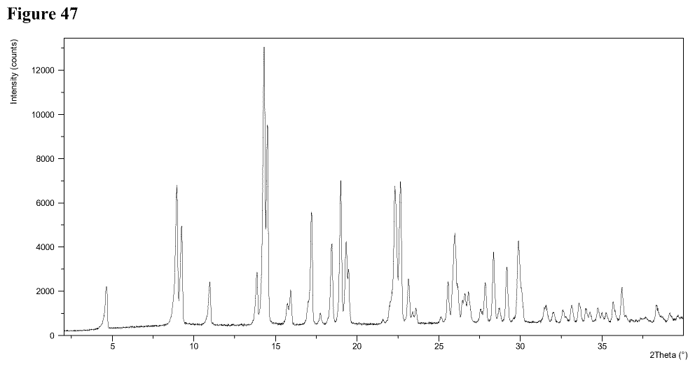

In some embodiments, the crystalline morpholine salt of the compound of Formula I has an XRPD profile substantially as shown in FIG. 47. In some embodiments, the crystalline morpholine salt of the compound of Formula I has an XRPD pattern displaying at least two, at least three, at least four, at least five, or at least six of the degree 2θ-reflections with the greatest intensity as the XRPD pattern substantially as shown in FIG. 47.

In some embodiments, the crystalline morpholine salt of the compound of Formula I has an XRPD pattern comprising 2θ-reflections (+/−0.2 degrees 2θ) at 14.3°, 19.0°, and 22.7°. In some embodiments, the crystalline morpholine salt of the compound of Formula I has an XRPD pattern comprising 2θ-reflections (+/−0.2 degrees 2θ) at 14.3°, 19.0°, and 22.7°, and one, two or three of the 2θ-reflections (+/−0.2 degrees 2θ) at 9.0°, 14.5°, and 22.3°. In some embodiments, the crystalline morpholine salt of the compound of Formula I has an XRPD pattern comprising 2θ-reflections (+/−0.2 degrees 2θ) at 14.3°, 19.0°, and 22.7°, and one or two of the 2θ-reflections (+/−0.2 degrees 2θ) at 9.0°, 14.5°, and 22.3°. In some embodiments, the crystalline morpholine salt of the compound of Formula I has an XRPD pattern comprising 2θ-reflections (+/−0.2 degrees 2θ) at 14.3°, 19.0°, and 22.7°, and one of the 2θ-reflections (+/−0.2 degrees 2θ) at 9.0°, 14.5°, and 22.3°. In some embodiments, the crystalline morpholine salt of the compound of Formula I has an XRPD pattern comprising 2θ-reflections (+/−0.2 degrees 2θ) at 14.3°, 19.0°, and 22.7°, and two of the 2θ-reflections (+/−0.2 degrees 2θ) at 9.0°, 14.5°, and 22.3°. In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°. In some embodiments, the crystalline morpholine salt of the compound of Formula I has an XRPD pattern comprising any three of the 2θ-reflections (+/−0.2 degrees 2θ) at 9.0°, 14.3°, 14.5°, 19.0°, 22.3°, and 22.7°.

In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°, and one, two or three of the 2θ-reflections (+/−0.2 degrees 2θ) at 4.7°, 17.2°, and 26.0°. In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°, and one or two of the 2θ-reflections (+/−0.2 degrees 2θ) at 4.7°, 17.2°, and 26.0°. In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°, and one of the 2θ-reflections (+/−0.2 degrees 2θ) at 4.7°, 17.2°, and 26.0°. In some embodiments, the crystalline morpholine salt of the compound of Formula I has an XRPD pattern comprising 2θ-reflections (+/−0.2 degrees 2θ) at 9.0°, 14.3°, 14.5°, 19.0°, 22.3°, and 22.7°, and two of the 2θ-reflections (+/−0.2 degrees 2θ) at 4.7°, 17.2°, and 26.0°. In some embodiments, the crystalline morpholine salt of the compound of Formula I has an XRPD pattern comprising 2θ-reflections (+/−0.2 degrees 2θ) at 4.7°, 9.0°, 14.3°, 14.5°, 17.2°, 19.0°, 22.3°, 22.7°, and 26.0°. In some embodiments, the crystalline morpholine salt of the compound of Formula I has an XRPD pattern comprising any three of the 2θ-reflections (+/−0.2 degrees 2θ) at 4.7°, 9.0°, 14.3°, 14.5°, 17.2°, 19.0°, 22.3°, 22.7°, and 26.0°.

In some embodiments, the crystalline morpholine salt of the compound of Formula I has an XRPD pattern comprising at least three of the 2θ-reflections (+/−0.2 degrees 2θ) at 4.7°, 9.0°, 14.3°, 14.5°, 17.2°, 19.0°, 22.3°, 22.7°, and 26.0°. In some embodiments, the crystalline morpholine salt of the compound of Formula I has an XRPD pattern comprising at least four of the 2θ-reflections (+/−0.2 degrees 2θ) at 4.7°, 9.0°, 14.3°, 14.5°, 17.2°, 19.0°, 22.3°, 22.7°, and 26.0°. In some embodiments, the crystalline morpholine salt of the compound of Formula I has an XRPD pattern comprising at least five of the 2θ-reflections (+/−0.2 degrees 2θ) at 4.7°, 9.0°, 14.3°, 14.5°, 17.2°, 19.0°, 22.3°, 22.7°, and 26.0°.

In some embodiments, the crystalline morpholine salt of the compound of Formula I has an XRPD pattern comprising peaks at:

| Pos. | Rel. Int. |
|---|---|
| [°2Th.] | [%] |
| 4.7 | 15 |
| 9.0 | 47 |
| 9.3 | 32 |
| 11.0 | 15 |
| 13.9 | 18 |
| 14.3 | 100 |
| 14.5 | 69 |
| 17.2 | 40 |
| 18.5 | 28 |
| 19.0 | 49 |
| 19.3 | 28 |
| 22.3 | 47 |
| 22.7 | 50 |
| 26.0 | 31 |
| 28.4 | 24 |
| 29.2 | 19 |
| 29.9 | 28 |
| 36.2 | 11 |

### Compound of Formula I, L-Arginine Salt

In some embodiments, the compound of Formula I can be isolated as an L-arginine salt which can be amorphous or crystalline. In some embodiments, the L-arginine salt of the compound of Formula I is crystalline.

### Compound of Formula I, L-Arginine Salt, Form I

In some embodiments, the crystalline L-arginine salt of the compound of Formula I is a Form I (“compound of Formula I, L-arginine salt, Form I” or “Formula I, L-arginine, Form I”).

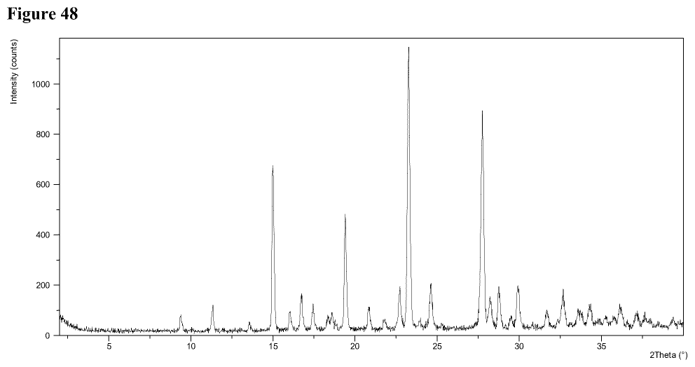

In some embodiments, Formula I, L-arginine salt, Form I has an XRPD profile substantially as shown in FIG. 48. In some embodiments, Formula I, L-arginine salt, Form I has an XRPD pattern displaying at least two, at least three, at least four, at least five, or at least six of the degree 2θ-reflections with the greatest intensity as the XRPD pattern substantially as shown in FIG. 48.

In some embodiments, Formula I, L-arginine salt, Form I has an XRPD pattern comprising 2θ-reflections (+/−0.2 degrees 2θ) at 15.0°, 23.3°, and 27.7°. In some embodiments, Formula I, L-arginine salt, Form I has an XRPD pattern comprising 2θ-reflections (+/−0.2 degrees 2θ) at 15.0°, 23.3°, and 27.7°, and one, two or three of the 2θ-reflections (+/−0.2 degrees 2θ) at 19.4°, 24.6°, and 29.9°. In some embodiments, Formula I, L-arginine salt, Form I has an XRPD pattern comprising 2θ-reflections (+/−0.2 degrees 2θ) at 15.0°, 23.3°, and 27.7°, and one or two of the 2θ-reflections (+/−0.2 degrees 2θ) at 19.4°, 24.6°, and 29.9°. In some embodiments, Formula I, L-arginine salt, Form I has an XRPD pattern comprising 2θ-reflections (+/−0.2 degrees 2θ) at 15.0°, 23.3°, and 27.7°, and one of the 2θ-reflections (+/−0.2 degrees 2θ) at 19.4°, 24.6°, and 29.9°. In some embodiments, Formula I, L-arginine salt, Form I has an XRPD pattern comprising 2θ-reflections (+/−0.2 degrees 2θ) at 15.0°, 23.3°, and 27.7°, and two of the 2θ-reflections (+/−0.2 degrees 2θ) at 19.4°, 24.6°, and 29.9°. In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°. In some embodiments, Formula I, L-arginine salt, Form I has an XRPD pattern comprising any three of the 2θ-reflections (+/−0.2 degrees 2θ) at 15.0°, 19.4°, 23.3°, 24.6°, 27.7°, and 29.9°.

In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°, and one, two or three of the 2θ-reflections (+/−0.2 degrees 2θ) at 11.3°, 16.7°, and 22.7°. In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°, and one or two of the 20-reflections (+/−0.2 degrees 2θ) at 11.3°, 16.7°, and 22.7°. In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°, and one of the 2θ-reflections (+/−0.2 degrees 2θ) at 11.3°, 16.7°, and 22.7°. In some embodiments, Formula I, L-arginine salt, Form I has an XRPD pattern comprising 2θ-reflections (+/−0.2 degrees 2θ) at 15.0°, 19.4°, 23.3°, 24.6°, 27.7°, and 29.9°, and two of the 2θ-reflections (+/−0.2 degrees 2θ) at 11.3°, 16.7°, and 22.7°. In some embodiments, Formula I, L-arginine salt, Form I has an XRPD pattern comprising 2θ-reflections (+/−0.2 degrees 2θ) at 11.3°, 15.0°, 16.7°, 19.4°, 22.7°, 23.3°, 24.6°, 27.7°, and 29.9°. In some embodiments, Formula I, L-arginine salt, Form I has an XRPD pattern comprising any three of the 2θ-reflections (+/−0.2 degrees 2θ) at 11.3°, 15.0°, 16.7°, 19.4°, 22.7°, 23.3°, 24.6°, 27.7°, and 29.9°.

In some embodiments, Formula I, L-arginine salt, Form I has an XRPD pattern comprising at least three of the 2θ-reflections (+/−0.2 degrees 2θ) at 11.3°, 15.0°, 16.7°, 19.4°, 22.7°, 23.3°, 24.6°, 27.7°, and 29.9°. In some embodiments, Formula I, L-arginine salt, Form I has an XRPD pattern comprising at least four of the 2θ-reflections (+/−0.2 degrees 2θ) at 11.3°, 15.0°, 16.7°, 19.4°, 22.7°, 23.3°, 24.6°, 27.7°, and 29.9°. In some embodiments, Formula I, L-arginine salt, Form I has an XRPD pattern comprising at least five of the 2θ-reflections (+/−0.2 degrees 2θ) at 11.3°, 15.0°, 16.7°, 19.4°, 22.7°, 23.3°, 24.6°, 27.7°, and 29.9°.

In some embodiments, Formula I, L-arginine salt, Form I has an XRPD pattern comprising peaks at:

| Pos. | Rel. Int. |
|---|---|
| [°2Th.] | [%] |
| 11.3 | 8 |
| 15.0 | 54 |
| 16.0 | 7 |
| 16.7 | 12 |
| 17.4 | 8 |
| 18.3 | 4 |
| 19.4 | 35 |
| 20.8 | 8 |
| 22.7 | 13 |
| 23.3 | 100 |
| 24.6 | 15 |
| 27.7 | 58 |
| 28.7 | 14 |
| 29.9 | 15 |
| 32.7 | 11 |
| 33.6 | 4 |
| 36.1 | 6 |

### Compound of Formula I, L-Arginine Salt, Form II

In some embodiments, the crystalline L-arginine salt of the compound of Formula I is a Form II (“compound of Formula I, L-arginine salt, Form II” or “Formula I, L-arginine, Form II”).

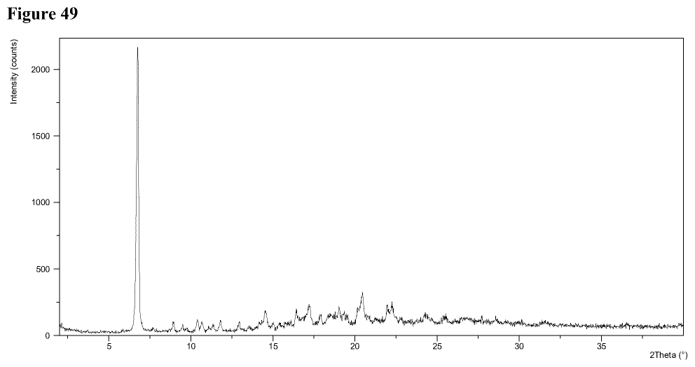

In some embodiments, Formula I, L-arginine salt, Form II has an XRPD profile substantially as shown in FIG. 49. In some embodiments, Formula I, L-arginine salt, Form II has an XRPD pattern displaying at least two, at least three, at least four, at least five, or at least six of the degree 2θ-reflections with the greatest intensity as the XRPD pattern substantially as shown in FIG. 49.

In some embodiments, Formula I, L-arginine salt, Form II has an XRPD pattern comprising 2θ-reflections (+/−0.2 degrees 2θ) at 6.8°, 14.5°, and 20.5°. In some embodiments, Formula I, L-arginine salt, Form II has an XRPD pattern comprising 2θ-reflections (+/−0.2 degrees 2θ) at 6.8°, 14.5°, and 20.5°, and one, two or three of the 2θ-reflections (+/−0.2 degrees 2θ) at 8.9°, 11.8°, and 17.2°. In some embodiments, Formula I, L-arginine salt, Form II has an XRPD pattern comprising 2θ-reflections (+/−0.2 degrees 2θ) at 6.8°, 14.5°, and 20.5°, and one or two of the 2θ-reflections (+/−0.2 degrees 2θ) at 8.9°, 11.8°, and 17.2°. In some embodiments, Formula I, L-arginine salt, Form II has an XRPD pattern comprising 2θ-reflections (+/−0.2 degrees 2θ) at 6.8°, 14.5°, and 20.5°, and one of the 2θ-reflections (+/−0.2 degrees 2θ) at 8.9°, 11.8°, and 17.2°. In some embodiments, Formula I, L-arginine salt, Form II has an XRPD pattern comprising 2θ-reflections (+/−0.2 degrees 2θ) at 6.8°, 14.5°, and 20.5°, and two of the 20-reflections (+/−0.2 degrees 2θ) at 8.9°, 11.8°, and 17.2°. In some embodiments, Formula I, L-arginine salt, Form II has an XRPD pattern comprising 2θ-reflections (+/−0.2 degrees 2θ) at 6.8°, 8.9°, 11.8°, 14.5°, 17.2°, and 20.5°. In some embodiments, Formula I, L-arginine salt, Form II has an XRPD pattern comprising any three of the 2θ-reflections (+/−0.2 degrees 2θ) at 6.8°, 8.9°, 11.8°, 14.5°, 17.2°, and 20.5°.

In some embodiments, Formula I, L-arginine salt, Form II has an XRPD pattern comprising at least three of the 2θ-reflections (+/−0.2 degrees 2θ) at 6.8°, 8.9°, 11.8°, 14.5°, 17.2°, and 20.5°. In some embodiments, Formula I, L-arginine salt, Form II has an XRPD pattern comprising at least four of the 2θ-reflections (+/−0.2 degrees 2θ) at 6.8°, 8.9°, 11.8°, 14.5°, 17.2°, and 20.5°. In some embodiments, Formula I, L-arginine salt, Form II has an XRPD pattern comprising at least five of the 2θ-reflections (+/−0.2 degrees 2θ) at 6.8°, 8.9°, 11.8°, 14.5°, 17.2°, and 20.5°.

In some embodiments, Formula I, L-arginine salt, Form II has an XRPD pattern comprising peaks at:

| Pos. | Rel. Int. |
|---|---|
| [°2Th.] | [%] |
| 6.8 | 100 |
| 8.9 | 3 |
| 10.5 | 2 |
| 11.8 | 4 |
| 12.9 | 3 |
| 14.5 | 6 |
| 17.2 | 7 |
| 17.9 | 3 |
| 19.2 | 2 |
| 20.5 | 11 |
| 22.1 | 4 |
| 24.4 | 2 |
| 25.5 | 2 |

### Compound of Formula I, L-Arginine Salt, Form III

In some embodiments, the crystalline L-arginine salt of the compound of Formula I is a Form III (“compound of Formula I, L-arginine salt, Form III” or “Formula I, L-arginine, Form III”).

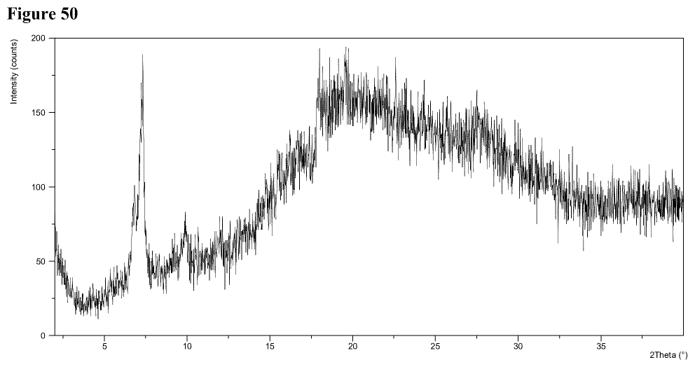

In some embodiments, Formula I, L-arginine salt, Form III has an XRPD profile substantially as shown in FIG. 50. In some embodiments, Formula I, L-arginine salt, Form III has an XRPD pattern displaying at least two, at least three, at least four, at least five, or at least six of the degree 2θ-reflections with the greatest intensity as the XRPD pattern substantially as shown in FIG. 50.

In some embodiments, Formula I, L-arginine salt, Form III has an XRPD pattern comprising a 2θ-reflection (+/−0.2 degrees 2θ) at 7.3°. In some embodiments, Formula I, L-arginine salt, Form III has an XRPD pattern comprising 2θ-reflections (+/−0.2 degrees 2θ) at 7.3° and 9.6°.

In some embodiments, Formula I, L-arginine salt, Form II has an XRPD pattern comprising peaks at:

| Pos. | Rel. Int. |
|---|---|
| [°2Th.] | [%] |
| 7.3 | 100 |
| 9.6 | 10 |

### Compound of Formula I, Co-Crystal

In some embodiments, the compound of Formula I can be isolated as a co-crystal. In some embodiments, the compound of Formula I co-crystal is crystalline.

### Compound of Formula I, Trans-Ferulic Co-Crystal

In some embodiments, the compound of Formula I can be isolated as a trans-ferulic acid salt or co-crystal. In some embodiments, the compound of Formula I can be isolated as a trans-ferulic acid co-crystal (“compound of Formula I trans-ferulic co-crystal” or “Formula I trans-ferulic co-crystal”).

In some embodiments, the compound of Formula I trans-ferulic co-crystal is crystalline.

### Compound of Formula I, Trans-Ferulic Co-Crystal, Form I

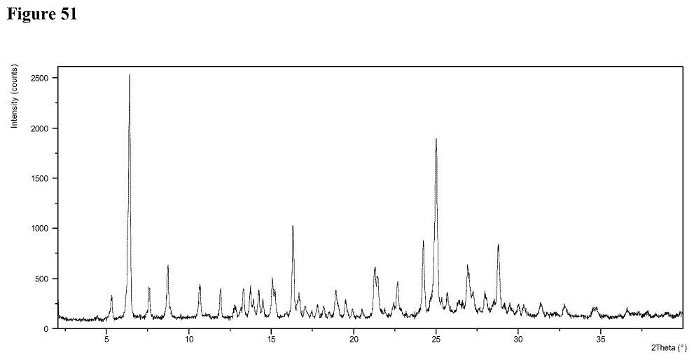

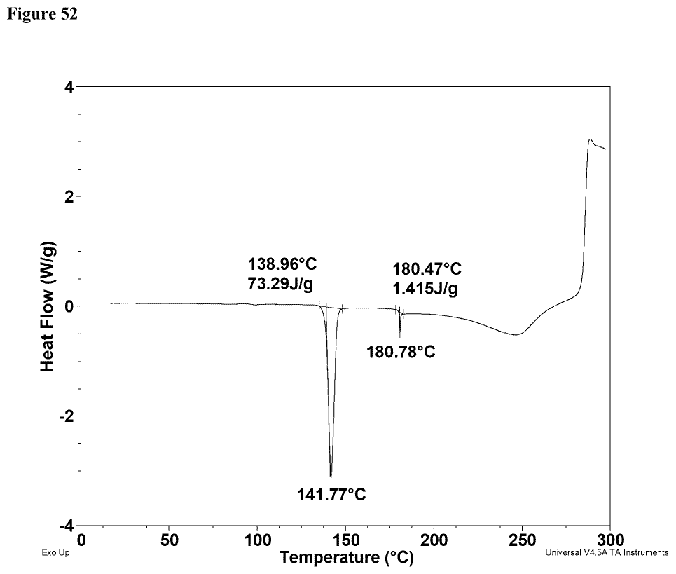

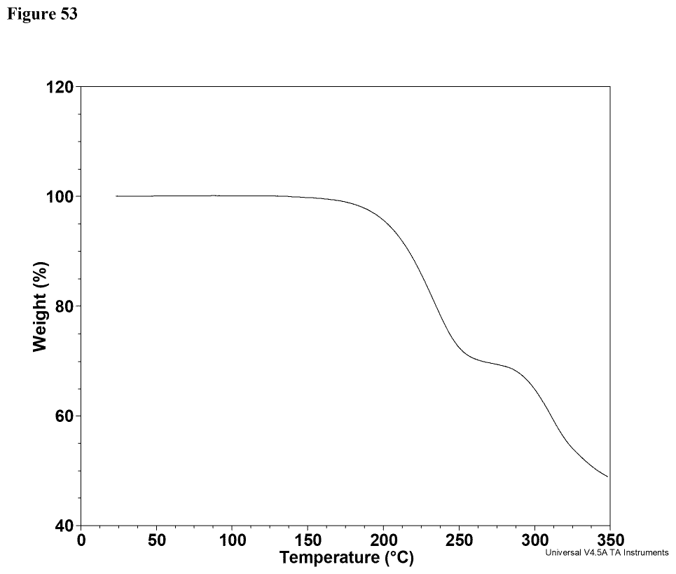

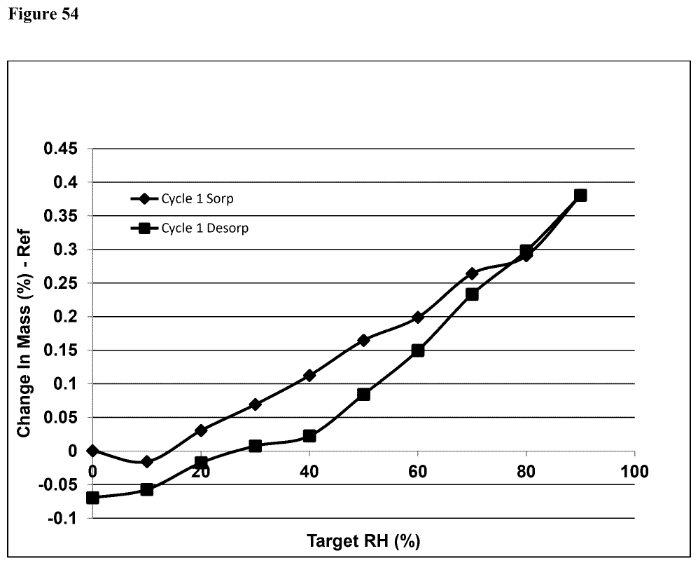

In some embodiments, the crystalline compound of Formula I, trans-ferulic co-crystal is a Form I (“compound of Formula I, trans-ferulic co-crystal, Form I” or “Formula I trans-ferulic co-crystal, Form I”). In some embodiments, compound of Formula I, trans-ferulic co-crystal, Form I has an XRPD profile substantially as shown in FIG. 51. The Formula I trans-ferulic co-crystal, Form I may exhibit a DSC thermogram substantially as shown in FIG. 52. The Formula I trans-ferulic co-crystal, Form I may exhibit a TGA graph substantially as shown in FIG. 53. The Formula I trans-ferulic co-crystal, Form I may exhibit a DVS curve substantially as shown in FIG. 54.

In some embodiments of the Formula I trans-ferulic co-crystal, Form I, at least one, at least two, at least three, or all of the following (a)-(d) apply: (a) the Formula I trans-ferulic co-crystal, Form I has an XRPD profile substantially as shown in FIG. 51; (b) the Formula I trans-ferulic co-crystal, Form I exhibits a DSC thermogram substantially as shown in FIG. 52; (c) the Formula I trans-ferulic co-crystal, Form I exhibits a TGA graph substantially as shown in FIG. 53; (d) the Formula I trans-ferulic co-crystal, Form I exhibits a DVS curve substantially as shown in FIG. 54.

In some embodiments, the Formula I trans-ferulic co-crystal, Form I has an XRPD pattern displaying at least two, at least three, at least four, at least five, or at least six of the degree 2θ-reflections with the greatest intensity as the XRPD pattern substantially as shown in FIG. 51.

In some embodiments, the Formula I trans-ferulic co-crystal, Form I has an XRPD pattern comprising 2θ-reflections (+/−0.2 degrees 2θ) at 6.4°, 16.3°, and 25.0°. In some embodiments, the Formula I trans-ferulic co-crystal, Form I has an XRPD pattern comprising 2θ-reflections (+/−0.2 degrees 2θ) at 6.4°, 16.3°, and 25.0°, and one, two or three of the 20-reflections (+/−0.2 degrees 2θ) at 8.7°, 24.2°, and 28.8°. In some embodiments, the Formula I trans-ferulic co-crystal, Form I has an XRPD pattern comprising 2θ-reflections (+/−0.2 degrees 2θ) at 6.4°, 16.3°, and 25.0°, and one or two of the 2θ-reflections (+/−0.2 degrees 2θ) at 8.7°, 24.2°, and 28.8°. In some embodiments, Formula I trans-ferulic co-crystal, Form I has an XRPD pattern comprising 2θ-reflections (+/−0.2 degrees 2θ) at 6.4°, 16.3°, and 25.0°, and one of the 2θ-reflections (+/−0.2 degrees 2θ) at 8.7°, 24.2°, and 28.8°. In some embodiments, Formula I trans-ferulic co-crystal, Form I has an XRPD pattern comprising 2θ-reflections (+/−0.2 degrees 2θ) at 6.4°, 16.3°, and 25.0°, and two of the 2θ-reflections (+/−0.2 degrees 2θ) at 8.7°, 24.2°, and 28.8°. In some embodiments, Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°. In some embodiments, Formula I trans-ferulic co-crystal, Form I has an XRPD pattern comprising any three of the 2θ-reflections (+/−0.2 degrees 2θ) at 6.4°, 8.7°, 16.3°, 24.2°, 25.0°, and 28.8°.

In some embodiments, Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°, and one, two or three of the 2θ-reflections (+/−0.2 degrees 2θ) at 21.3°, 22.7°, and 26.9°. In some embodiments, Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°, and one or two of the 2θ-reflections (+/−0.2 degrees 2θ) at 21.3°, 22.7°, and 26.9°. In some embodiments, Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°, and one of the 2θ-reflections (+/−0.2 degrees 2θ) at 21.3°, 22.7°, and 26.9°. In some embodiments Formula I trans-ferulic co-crystal, Form I has an XRPD pattern comprising 2θ-reflections (+/−0.2 degrees 2θ) at 6.4°, 8.7°, 16.3°, 24.2°, 25.0°, and 28.8°, and two of the 2θ-reflections (+/−0.2 degrees 2θ) at 21.3°, 22.7°, and 26.9°. In some embodiments, Formula I trans-ferulic co-crystal, Form I has an XRPD pattern comprising 2θ-reflections (+/−0.2 degrees 2θ) at 6.4°, 8.7°, 16.3°, 21.3°, 22.7°, 24.2°, 25.0°, 26.9° and 28.8°. In some embodiments, Formula I trans-ferulic co-crystal, Form I has an XRPD pattern comprising any three of the 2θ-reflections (+/−0.2 degrees 2θ) at 6.4°, 8.7°, 16.3°, 21.3°, 22.7°, 24.2°, 25.0°, 26.9° and 28.8°.

In some embodiments, Formula I trans-ferulic co-crystal, Form I has an XRPD pattern comprising at least three of the 2θ-reflections (+/−0.2 degrees 2θ) at 6.4°, 8.7°, 10.7°, 14.2°, 15.10, 16.3°, 18.9°, 21.3°, 22.7°, 24.2°, 25.0°, 26.9° and 28.8°. In some embodiments, Formula I trans-ferulic co-crystal, Form I has an XRPD pattern comprising at least four of the 20-reflections (+/−0.2 degrees 2θ) at 6.4°, 8.7°, 10.7°, 14.2°, 15.10, 16.3°, 18.9°, 21.3°, 22.7°, 24.2°, 25.0°, 26.9° and 28.8°. In some embodiments Formula I trans-ferulic co-crystal, Form I has an XRPD pattern comprising at least five of the 2θ-reflections (+/−0.2 degrees 2θ) at 6.4°, 8.7°, 10.7°, 14.2°, 15.1°, 16.3°, 18.9°, 21.3°, 22.7°, 24.2°, 25.0°, 26.9° and 28.8°.

In some embodiments, Formula I trans-ferulic co-crystal, Form I has an XRPD pattern comprising peaks at:

| Pos. | Rel. Int. |
|---|---|
| [°2Th.] | [%] |
| 6.4 | 100 |
| 7.6 | 9 |
| 8.7 | 23 |
| 10.7 | 14 |
| 13.7 | 9 |
| 14.2 | 11 |
| 15.1 | 14 |
| 16.3 | 38 |
| 18.9 | 10 |
| 19.5 | 6 |
| 21.3 | 20 |
| 22.7 | 15 |
| 24.2 | 31 |
| 25.0 | 74 |
| 26.9 | 19 |
| 28.0 | 7 |
| 28.8 | 28 |

In some embodiments, Formula I trans-ferulic co-crystal, Form I is characterized by a DSC thermogram substantially as shown in FIG. 52.

In some embodiments, Formula I trans-ferulic co-crystal, Form I is characterized by a DSC thermogram having one or both of (i) an endothermic transition at about 139° C. and (ii) an endothermic transition at about 180° C. In some embodiments, Formula I trans-ferulic co-crystal, Form I is characterized by a DSC thermogram having (i) an endothermic transition at about 139° C. and (ii) an endothermic transition at about 180° C.

In some embodiments, Formula I trans-ferulic co-crystal, Form I is characterized by a TGA curve substantially as shown in FIG. 53. In some embodiments, Formula I trans-ferulic co-crystal, Form I loses mass starting at about 115° C.

In some embodiments Formula I trans-ferulic co-crystal, Form I is characterized by a DVS curve substantially as shown in shown in FIG. 54. In some embodiments, Formula I trans-ferulic co-crystal, Form I absorbs less than about 0.35% of water up to 90% RH at 25° C.

In some embodiments, Formula I trans-ferulic co-crystal, Form I has the following properties:
(a) an XRPD pattern substantially as shown in FIG. 51;
(b) a DSC thermogram substantially as shown in FIG. 52;
(c) a TGA curve substantially as shown in FIG. 53; and
(d) a DVS curve substantially as shown in FIG. 54.

### Compound of Formula I, Trans-Ferulic Co-Crystal, Form II

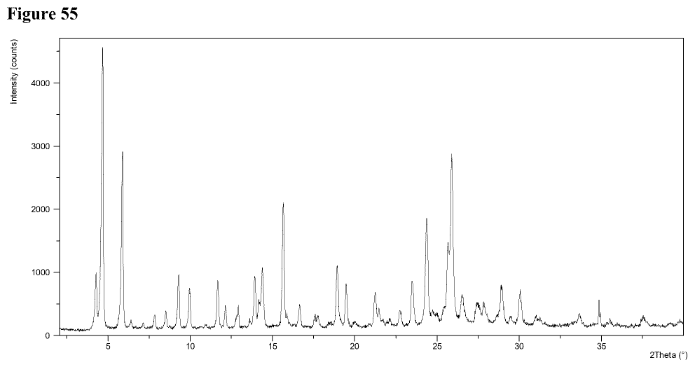

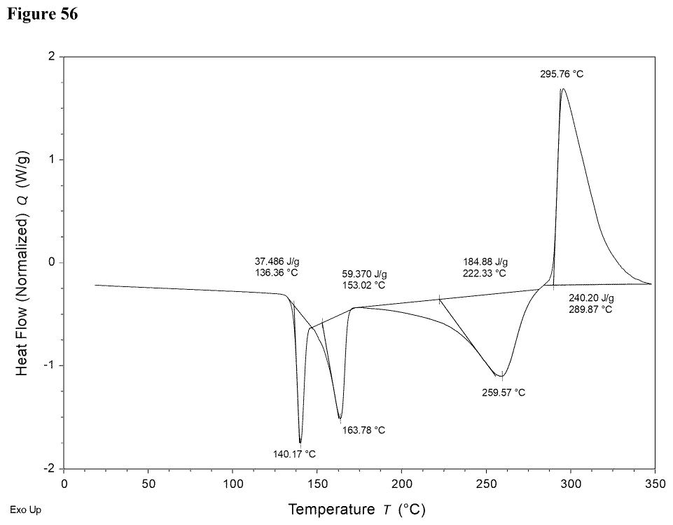

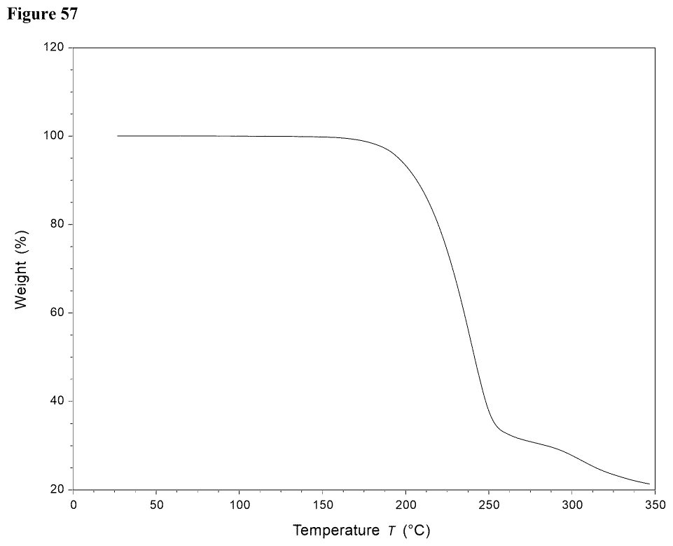

In some embodiments, the crystalline compound of Formula I, trans-ferulic co-crystal is a Form II (“compound of Formula I, trans-ferulic co-crystal, Form II” or “Formula I trans-ferulic co-crystal, Form II”). In some embodiments, compound of Formula I, trans-ferulic co-crystal, Form II has an XRPD profile substantially as shown in FIG. 55. The Formula I trans-ferulic co-crystal, Form II may exhibit a DSC thermogram substantially as shown in FIG. 56. The Formula I trans-ferulic co-crystal, Form II may exhibit a TGA graph substantially as shown in FIG. 57.

In some embodiments of the Formula I trans-ferulic co-crystal, Form II, at least one, at least two, at least three, or all of the following (a)-(c) apply: (a) the Formula I trans-ferulic co-crystal, Form II has an XRPD profile substantially as shown in FIG. 55; (b) the Formula I trans-ferulic co-crystal, Form II exhibits a DSC thermogram substantially as shown in FIG. 56; (c) the Formula I trans-ferulic co-crystal, Form I exhibits a TGA graph substantially as shown in FIG. 57.

In some embodiments, the Formula I trans-ferulic co-crystal, Form II has an XRPD pattern displaying at least two, at least three, at least four, at least five, or at least six of the degree 2θ-reflections with the greatest intensity as the XRPD pattern substantially as shown in FIG. 55.

In some embodiments, the Formula I trans-ferulic co-crystal, Form II has an XRPD pattern comprising 2θ-reflections (+/−0.2 degrees 2θ) at 4.7°, 5.9°, and 25.9°. In some embodiments, the Formula I trans-ferulic co-crystal, Form II has an XRPD pattern comprising 2θ-reflections (+/−0.2 degrees 2θ) at 4.7°, 5.9°, and 25.9°, and one, two or three of the 20-reflections (+/−0.2 degrees 2θ) at 15.7°, 18.9°, and 24.4°. In some embodiments, the Formula I trans-ferulic co-crystal, Form II has an XRPD pattern comprising 2θ-reflections (+/−0.2 degrees 2θ) at 4.7°, 5.9°, and 25.9°, and one or two of the 2θ-reflections (+/−0.2 degrees 2θ) at 15.7°, 18.9°, and 24.4°. In some embodiments, Formula I trans-ferulic co-crystal, Form II has an XRPD pattern comprising 2θ-reflections (+/−0.2 degrees 2θ) at 4.7°, 5.9°, and 25.9°, and one of the 2θ-reflections (+/−0.2 degrees 2θ) at 15.7°, 18.9°, and 24.4°. In some embodiments, Formula I trans-ferulic co-crystal, Form II has an XRPD pattern comprising 2θ-reflections (+/−0.2 degrees 2θ) at 4.7°, 5.9°, and 25.9°, and two of the 2θ-reflections (+/−0.2 degrees 2θ) at 15.7°, 18.9°, and 24.4°. In some embodiments, Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°. In some embodiments, Formula I trans-ferulic co-crystal, Form II has an XRPD pattern comprising any three of the 2θ-reflections (+/−0.2 degrees 2θ) at 4.7°, 5.9°, 15.7°, 18.9°, 24.4°, and 25.9°.

In some embodiments, Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°, and one, two or three of the 2θ-reflections (+/−0.2 degrees 2θ) at 4.3°, 9.3°, and 14.4°. In some embodiments, Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°, and one or two of the 2θ-reflections (+/−0.2 degrees 2θ) at 4.3°, 9.3°, and 14.4°. In some embodiments, Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°, and one of the 2θ-reflections (+/−0.2 degrees 2θ) at 4.3°, 9.3°, and 14.4°. In some embodiments Formula I trans-ferulic co-crystal, Form II has an XRPD pattern comprising 2θ-reflections (+/−0.2 degrees 2θ) at 4.7°, 5.9°, 15.7°, 18.9°, 24.4°, and 25.9°, and two of the 2θ-reflections (+/−0.2 degrees 2θ) at 4.3°, 9.3°, and 14.4°. In some embodiments, Formula I trans-ferulic co-crystal, Form II has an XRPD pattern comprising 2θ-reflections (+/−0.2 degrees 2θ) at 4.3°, 4.7°, 5.9°, 9.3°, 14.4°, 15.7°, 18.9°, 24.4°, and 25.9°. In some embodiments, Formula I trans-ferulic co-crystal, Form II has an XRPD pattern comprising any three of the 2θ-reflections (+/−0.2 degrees 2θ) at 4.3°, 4.7°, 5.9°, 9.3°, 14.4°, 15.7°, 18.9°, 24.4°, and 25.9°.

In some embodiments, Formula I trans-ferulic co-crystal, Form II has an XRPD pattern comprising at least three of the 2θ-reflections (+/−0.2 degrees 2θ) at 4.3°, 4.7°, 5.9°, 9.3°, 14.4°, 15.7°, 18.9°, 24.4°, and 25.9°. In some embodiments, Formula I trans-ferulic co-crystal, Form II has an XRPD pattern comprising at least four of the 2θ-reflections (+/−0.2 degrees 2θ) at 4.3°, 4.7°, 5.9°, 9.3°, 14.4°, 15.7°, 18.9°, 24.4°, and 25.9°. In some embodiments Formula I trans-ferulic co-crystal, Form II has an XRPD pattern comprising at least five of the 2θ-reflections (+/−0.2 degrees 2θ) at 4.3°, 4.7°, 5.9°, 9.3°, 14.4°, 15.7°, 18.9°, 24.4°, and 25.9°.

In some embodiments, Formula I trans-ferulic co-crystal, Form II has an XRPD pattern comprising peaks at:

| Pos. | Rel. Int. |
|---|---|
| [°2Th.] | [%] |
| 4.3 | 20 |
| 4.7 | 100 |
| 5.9 | 63 |
| 9.3 | 19 |
| 11.7 | 17 |
| 14.4 | 21 |
| 15.7 | 45 |
| 16.7 | 8 |
| 18.9 | 22 |
| 19.5 | 15 |
| 21.3 | 6 |
| 23.5 | 16 |
| 24.4 | 37 |
| 25.9 | 60 |
| 27.4 | 6 |
| 28.9 | 13 |
| 30.0 | 11 |

In some embodiments, Formula I trans-ferulic co-crystal, Form II is characterized by a DSC thermogram substantially as shown in FIG. 56.

In some embodiments, Formula I trans-ferulic co-crystal, Form II is characterized by a DSC thermogram having at least one of (i) an endothermic transition at about 136° C., (ii) an endothermic transition at about 153° C., (iii) an endothermic transition at about 222° C., and (iv) an exothermic transition at about 290° C. In some embodiments, Formula I trans-ferulic co-crystal, Form II is characterized by a DSC thermogram having (i) an endothermic transition at about 136° C., (ii) an endothermic transition at about 153° C., (iii) an endothermic transition at about 222° C., and (iv) an exothermic transition at about 290° C.

In some embodiments, Formula I trans-ferulic co-crystal, Form II is characterized by a TGA curve substantially as shown in FIG. 57. In some embodiments, Formula I trans-ferulic co-crystal, Form II is characterized in being unsolvated.

In some embodiments, Formula I trans-ferulic co-crystal, Form II has the following properties:
(a) an XRPD pattern substantially as shown in FIG. 55;
(b) a DSC thermogram substantially as shown in FIG. 56; and
(c) a TGA curve substantially as shown in FIG. 57.

### Compound of Formula I, Tromethamine Co-Crystal

In some embodiments, the compound of Formula I can be isolated as a tromethamine salt or co-crystal. In some embodiments, the compound of Formula I can be isolated as a tromethamine co-crystal (“compound of Formula I tromethamine co-crystal” or “Formula I tromethamine co-crystal”).

In some embodiments, the compound of Formula I tromethamine co-crystal is crystalline.

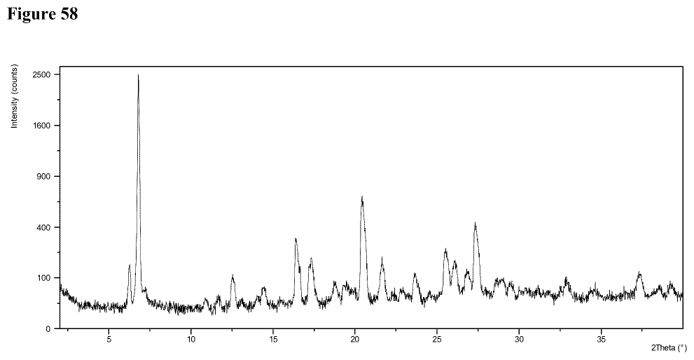

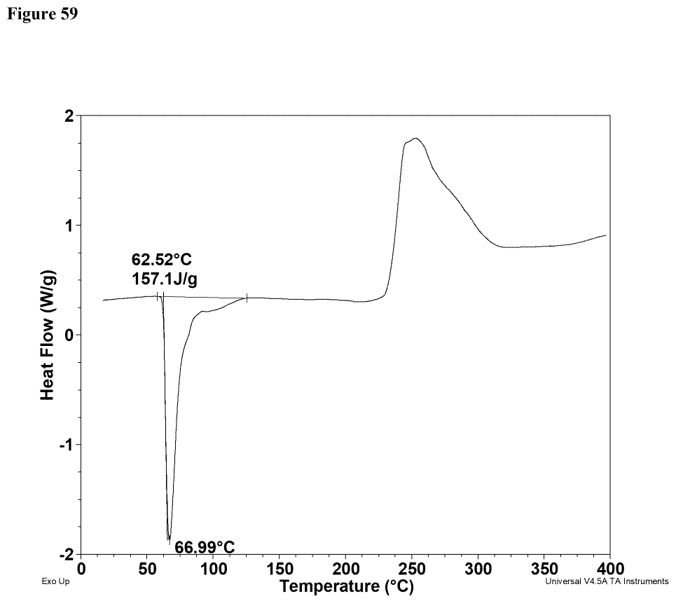

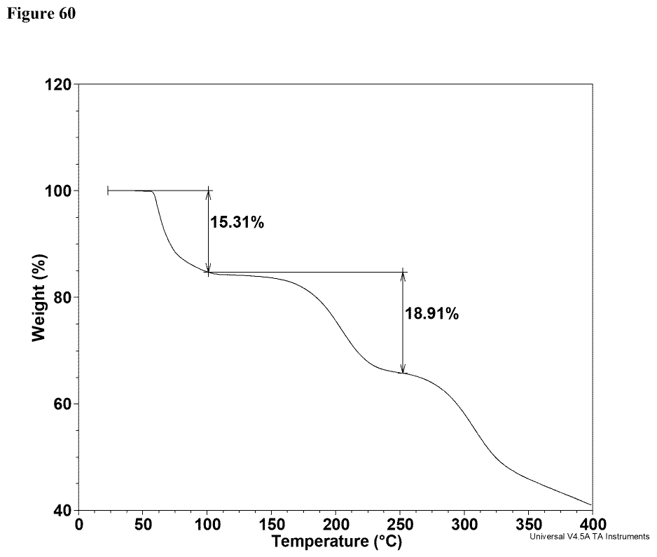

In some embodiments, the crystalline compound of Formula I, tromethamine co-crystal has an XRPD profile substantially as shown in FIG. 58. The crystalline compound of Formula I, tromethamine co-crystal may exhibit a DSC thermogram substantially as shown in FIG. 59. The crystalline compound of Formula I, tromethamine co-crystal may exhibit a TGA graph substantially as shown in FIG. 60.

In some embodiments of the crystalline compound of Formula I, tromethamine co-crystal, at least one, at least two, at least three, or all of the following (a)-(c) apply: (a) the crystalline compound of Formula I, tromethamine co-crystal has an XRPD profile substantially as shown in FIG. 58; (b) the crystalline compound of Formula I, tromethamine co-crystal exhibits a DSC thermogram substantially as shown in FIG. 59; (c) the crystalline compound of Formula I, tromethamine co-crystal exhibits a TGA graph substantially as shown in FIG. 60.

In some embodiments, the crystalline compound of Formula I, tromethamine co-crystal has an XRPD pattern displaying at least two, at least three, at least four, at least five, or at least six of the degree 2θ-reflections with the greatest intensity as the XRPD pattern substantially as shown in FIG. 58.

In some embodiments, the crystalline compound of Formula I, tromethamine co-crystal has an XRPD pattern comprising 2θ-reflections (+/−0.2 degrees 2θ) at 6.8°, 20.4°, and 27.3°. In some embodiments, the crystalline compound of Formula I, tromethamine co-crystal has an XRPD pattern comprising 2θ-reflections (+/−0.2 degrees 2θ) at 6.8°, 20.4°, and 27.3°, and one, two or three of the 2θ-reflections (+/−0.2 degrees 2θ) at 16.4°, 17.3°, and 25.5°. In some embodiments, the crystalline compound of Formula I, tromethamine co-crystal has an XRPD pattern comprising 2θ-reflections (+/−0.2 degrees 2θ) at 6.8°, 20.4°, and 27.3°, and one or two of the 2θ-reflections (+/−0.2 degrees 2θ) at 16.4°, 17.3°, and 25.5°. In some embodiments, the crystalline compound of Formula I, tromethamine co-crystal has an XRPD pattern comprising 2θ-reflections (+/−0.2 degrees 2θ) at 6.8°, 20.4°, and 27.3°, and one of the 2θ-reflections (+/−0.2 degrees 2θ) at 16.4°, 17.3°, and 25.5°. In some embodiments, the crystalline compound of Formula I, tromethamine co-crystal has an XRPD pattern comprising 2θ-reflections (+/−0.2 degrees 2θ) at 6.8°, 20.4°, and 27.3°, and two of the 2θ-reflections (+/−0.2 degrees 2θ) at 16.4°, 17.3°, and 25.5°. In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°. In some embodiments, the crystalline compound of Formula I, tromethamine co-crystal has an XRPD pattern comprising any three of the 2θ-reflections (+/−0.2 degrees 2θ) at 6.8°, 16.4°, 17.3°, 20.4°, 25.5°, and 27.3°.

In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°, and one, two or three of the 2θ-reflections (+/−0.2 degrees 2θ) at 12.6°, 21.6°, and 23.7°. In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°, and one or two of the 2θ-reflections (+/−0.2 degrees 2θ) at 12.6°, 21.6°, and 23.7°. In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°, and one of the 2θ-reflections (+/−0.2 degrees 2θ) at 12.6°, 21.6°, and 23.7°. In some embodiments, the crystalline compound of Formula I, tromethamine co-crystal has an XRPD pattern comprising 2θ-reflections (+/−0.2 degrees 2θ) at 6.8°, 16.4°, 17.3°, 20.4°, 25.5°, and 27.3°, and two of the 2θ-reflections (+/−0.2 degrees 2θ) at 12.6°, 21.6°, and 23.7°. In some embodiments, the crystalline compound of Formula I, tromethamine co-crystal has an XRPD pattern comprising 2θ-reflections (+/−0.2 degrees 2θ) at 6.8°, 12.6°, 16.4°, 17.3°, 20.4°, 21.6°, 23.7°, 25.5°, and 27.3°. In some embodiments, the crystalline compound of Formula I, tromethamine co-crystal has an XRPD pattern comprising any three of the 2θ-reflections (+/−0.2 degrees 2θ) at 6.8°, 12.6°, 16.4°, 17.3°, 20.4°, 21.6°, 23.7°, 25.5°, and 27.3°.

In some embodiments, the crystalline compound of Formula I, tromethamine co-crystal has an XRPD pattern comprising at least three of the 2θ-reflections (+/−0.2 degrees 2θ) at 6.2°, 6.8°, 12.6°, 14.3°, 16.4°, 17.3°, 20.4°, 21.6°, 23.7°, 25.5°, 26.8°, 27.3°, 32.9°, and 37.3°. In some embodiments, the crystalline compound of Formula I, tromethamine co-crystal has an XRPD pattern comprising at least four of the 2θ-reflections (+/−0.2 degrees 2θ) at 6.2°, 6.8°, 12.6°, 14.3°, 16.4°, 17.3°, 20.4°, 21.6°, 23.7°, 25.5°, 26.8°, 27.3°, 32.9°, and 37.3°. In some embodiments, the crystalline compound of Formula I, tromethamine co-crystal has an XRPD pattern comprising at least five of the 2θ-reflections (+/−0.2 degrees 2θ) at 6.2°, 6.8°, 12.6°, 14.3°, 16.4°, 17.3°, 20.4°, 21.6°, 23.7°, 25.5°, 26.8°, 27.3°, 32.9°, and 37.3°.

In some embodiments, the crystalline compound of Formula I, tromethamine co-crystal has an XRPD pattern comprising peaks at:

| Pos | Rel. Int. |
|---|---|
| [°2Th.] | [%] |
| 6.2 | 5 |
| 6.8 | 100 |
| 12.6 | 3 |
| 14.3 | 1 |
| 16.4 | 12 |
| 17.3 | 6 |
| 20.4 | 24 |
| 21.6 | 6 |
| 23.7 | 3 |
| 25.5 | 7 |
| 26.8 | 3 |
| 27.3 | 15 |
| 32.9 | 2 |
| 37.3 | 3 |

In some embodiments, the crystalline compound of Formula I, tromethamine co-crystal is characterized by a DSC thermogram substantially as shown in FIG. 59.

In some embodiments, the crystalline compound of Formula I, tromethamine co-crystal is characterized by a DSC thermogram having endothermic transition with an onset at about 63° C.

In some embodiments, the crystalline compound of Formula I, tromethamine co-crystal is characterized by a TGA curve substantially as shown in FIG. 60. In some embodiments, the crystalline compound of Formula I, tromethamine co-crystal loses mass in multiple stages starting at about 50° C. and another stage starting at about 140° C.

In some embodiments, the crystalline compound of Formula I, tromethamine co-crystal has the following properties:
(a) an XRPD pattern substantially as shown in FIG. 58;
(b) a DSC thermogram substantially as shown in FIG. 59; and
(c) a TGA curve substantially as shown in FIG. 60.

### Methods of Use

In some embodiments, the salts, co-crystals, crystalline forms, and/or amorphous forms disclosed herein are used for treating or preventing an HIV infection in a subject. In some embodiments, the salts, co-crystals, crystalline forms, and/or amorphous forms disclosed herein are used for treating or preventing an HIV infection in a subject at risk for infection. In some embodiments, the salts, co-crystals, crystalline forms, and/or amorphous forms disclosed herein are used for pre-exposure prophylaxis (PrEP) to reduce the risk of sexually acquired HIV-1.

In some embodiments, a method for treating or preventing an HIV infection in a subject (e.g., a human), comprising administering a crystalline form, an amorphous form, a salt, or a co-crystal of the compound of Formula I disclosed herein, to the subject is disclosed.

In some embodiments, a method for inhibiting the replication of the HIV virus, treating AIDS or delaying the onset of AIDS in a subject (e.g., a human), comprising administering a crystalline form, an amorphous form, a salt, or a co-crystal of the compound of Formula I disclosed herein, to the subject is disclosed.

In some embodiments, a method for preventing an HIV infection in a subject (e.g., a human), comprising administering a crystalline form, an amorphous form, a salt, or a co-crystal of the compound of Formula I disclosed herein, to the subject is disclosed. In certain embodiments, the subject is at risk of contracting the HIV virus, such as a subject who has one or more risk factors known to be associated with contracting the HIV virus.

In some embodiments, a method for treating an HIV infection in a subject (e.g., a human), comprising administering a crystalline form, an amorphous form, a salt, or a co-crystal of the compound of Formula I disclosed herein, to the subject is disclosed.

In some embodiments, a method for treating an HIV infection in a subject (e.g., a human), comprising administering to the subject in need thereof a therapeutically effective amount of a crystalline form, an amorphous form, a salt, or a co-crystal of the compound of Formula I disclosed herein, in combination with a therapeutically effective amount of one or more (e.g., one, two, three, or four; or one or two; or one to three; or one to four) additional therapeutic agents selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof is disclosed.

In some embodiments, a method for treating an HIV infection in a subject (e.g., a human), comprising administering to the subject in need thereof a therapeutically effective amount of a crystalline form, an amorphous form, a salt, or a co-crystal of the compound of Formula I disclosed herein, in combination with a therapeutically effective amount of one or more (e.g., one, two, three, or four; or one or two; or one to three; or one to four) additional therapeutic agents selected from the group consisting of combination drug products for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MILK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof is disclosed.

In some embodiments, a method for treating an HIV infection in a subject (e.g., a human), comprising administering to the subject in need thereof a therapeutically effective amount of a pharmaceutically acceptable salt of Compound 1, or a co-crystal or crystalline form thereof, in combination with a therapeutically effective amount of one or more (e.g., one, two, three, or four; or one or two; or one to three; or one to four) additional therapeutic agents selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof is disclosed.

In certain embodiments, a method for treating an HIV infection in a subject (e.g., a human), comprising administering to the subject in need thereof a therapeutically effective amount of a crystalline form, an amorphous form, a salt, or a co-crystal of the compound of Formula I disclosed herein, in combination with a therapeutically effective amount of one or more (e.g., one, two, three, or four; or one or two; or one to three; or one to four) additional therapeutic agents selected from the group consisting of combination drug products for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MILK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof is disclosed.

In certain embodiments, a method for treating an HIV infection in a subject (e.g., a human), comprising administering to the subject in need thereof a therapeutically effective amount of a crystalline form, an amorphous form, a salt, or a co-crystal of the compound of Formula I disclosed herein, in combination with a therapeutically effective amount of one or more (e.g., one, two, three, or four; or one or two; or one to three; or one to four) additional therapeutic agents selected from the group consisting of combination drug products for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, and HIV nucleoside reverse transcriptase translocation inhibitors.

In certain embodiments, a pharmaceutically acceptable salt of a crystalline form, an amorphous form, a salt, or a co-crystal of the compound of Formula I disclosed herein, for use in medical therapy of an HIV infection (e.g., HIV-1 or the replication of the HIV virus (e.g., HIV-1) or AIDS or delaying the onset of AIDS in a subject (e.g., a human)) is disclosed.

In some embodiments, a pharmaceutically acceptable salt of a crystalline form, an amorphous form, a salt, or a co-crystal of the compound of Formula I disclosed herein, for use in medical therapy of an HIV infection (e.g., HIV-1 or the replication of the HIV virus (e.g., HIV-1) or AIDS or delaying the onset of AIDS in a subject (e.g., a human) is disclosed.

In certain embodiments, a pharmaceutically acceptable salt of a crystalline form, an amorphous form, a salt, or a co-crystal of the compound of Formula I disclosed herein, for use in the manufacture of a medicament for treating an HIV infection or the replication of the HIV virus or AIDS or delaying the onset of AIDS in a subject (e.g., a human) is disclosed. One embodiment relates to a pharmaceutically acceptable salt of a crystalline form, an amorphous form, a salt, or a co-crystal of the compound of Formula I disclosed herein, for use in the prophylactic or therapeutic treatment of an HIV infection or AIDS or for use in the therapeutic treatment or delaying the onset of AIDS.

In some embodiments, a pharmaceutically acceptable salt of a crystalline form, an amorphous form, a salt, or a co-crystal of the compound of Formula I disclosed herein for use in the manufacture of a medicament for treating an HIV infection or the replication of the HIV virus or AIDS or delaying the onset of AIDS in a subject (e.g., a human) is disclosed. One embodiment relates to a crystalline form, an amorphous form, a salt, or a co-crystal of the compound of Formula I disclosed herein, for use in the prophylactic or therapeutic treatment of an HIV infection or AIDS or for use in the therapeutic treatment or delaying the onset of AIDS.

In certain embodiments, the use of a crystalline form, an amorphous form, a salt, or a co-crystal of the compound of Formula I disclosed herein, for the manufacture of a medicament for an HIV infection in a subject (e.g., a human) is disclosed. In certain embodiments, a crystalline form, an amorphous form, a salt, or a co-crystal of the compound of Formula I disclosed herein, for use in the prophylactic or therapeutic treatment of an HIV infection is disclosed.

In certain embodiments, in the methods of use, the administration is to a subject (e.g., a human) in need of the treatment. In certain embodiments, in the methods of use, the administration is to a subject (e.g., a human) who is at risk of developing AIDS.

Disclosed herein is a crystalline form, an amorphous form, a salt, or a co-crystal of the compound of Formula I disclosed herein, for use in therapy. In one embodiment, the crystalline form, a salt, or a co-crystal of the compound of Formula I disclosed herein, is for use in a method of treating an HIV infection or the replication of the HIV virus or AIDS or delaying the onset of AIDS in a subject (e.g., a human).

In some embodiments, disclosed herein is a crystalline form, an amorphous form, a salt, or a co-crystal of the compound of Formula I disclosed herein, for use in therapy. In some embodiments, a crystalline form, an amorphous form, a salt, or a co-crystal of the compound of Formula I disclosed herein, is for use in a method of treating an HIV infection or the replication of the HIV virus or AIDS or delaying the onset of AIDS in a subject (e.g., a human).

Also disclosed herein is a crystalline form, an amorphous form, a salt, or a co-crystal of the compound of Formula I disclosed herein, for use in a method of treating or preventing HIV infection in a subject in need thereof. In certain embodiments, a crystalline form, an amorphous form, a salt, or a co-crystal of the compound of Formula I disclosed herein, for use in a method of treating HIV infection in a subject in need thereof is provided. In certain embodiments, the subject in need thereof is a human who has been infected with HIV. In certain embodiments, the subject in need thereof is a human who has been infected with HIV but who has not developed AIDS. In certain embodiments, the subject in need thereof is a subject at risk for developing AIDS. In certain embodiments, the subject in need thereof is a human who has been infected with HIV and who has developed AIDS.

In one embodiment, a crystalline form, an amorphous form, a salt, or a co-crystal of the compound of Formula I disclosed herein, in combination with one or more (e.g. one, two, three, or four; or one or two; or one to three; or one to four) additional therapeutic agents as described herein for use in a method of treating or preventing HIV infection in a subject in need thereof is provided. In one embodiment, said additional therapeutic agents are selected from the group consisting of combination drug products for HIV, other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MILK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, and HIV vaccines, or any combinations thereof. In one embodiment, said additional therapeutic agents are selected from the group consisting of HIV protease inhibiting compounds, HIV non-nucleoside inhibitors of reverse transcriptase, HIV non-nucleotide inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, pharmacokinetic enhancers, and other drugs for treating HIV, and combinations thereof.

In one embodiment, a crystalline form, an amorphous form, a salt, or a co-crystal of the compound of Formula I disclosed herein, in combination with a first additional therapeutic agent selected from the group consisting of tenofovir alafenamide fumarate, tenofovir alafenamide, and tenofovir alafenamide hemifumarate, and a second additional therapeutic agent, wherein the second additional therapeutic agent is emtricitabine, is provided for use in a method of treating or preventing HIV infection in a subject in need thereof. In a particular embodiment, a crystalline form, an amorphous form, a salt, or a co-crystal of the compound of Formula I disclosed herein, in combination with a first additional therapeutic agent selected from the group consisting of tenofovir disoproxil fumarate, tenofovir disoproxil, and tenofovir disoproxil hemifumarate, and a second additional therapeutic agent, wherein the second additional therapeutic agent is emtricitabine, is provided for use in a method of treating or preventing HIV infection in a subject in need thereof.

In some embodiments, a crystalline form, an amorphous form, a salt, or a co-crystal of the compound of Formula I disclosed herein, in combination with a first additional therapeutic agent selected from the group consisting of tenofovir alafenamide fumarate, tenofovir alafenamide, and tenofovir alafenamide hemifumarate, and a second additional therapeutic agent, wherein the second additional therapeutic agent is emtricitabine, is provided for use in a method of treating or preventing HIV infection in a subject in need thereof. In a particular embodiment, a crystalline form, an amorphous form, a salt, or a co-crystal of the compound of Formula I disclosed herein, in combination with a first additional therapeutic agent selected from the group consisting of tenofovir disoproxil fumarate, tenofovir disoproxil, and tenofovir disoproxil hemifumarate, and a second additional therapeutic agent, wherein the second additional therapeutic agent is emtricitabine, is provided for use in a method of treating or preventing HIV infection in a subject in need thereof.

In some embodiments, a crystalline form, an amorphous form, a salt, or a co-crystal of the compound of Formula I disclosed herein, is provided in combination with at least one additional therapeutic agent selected from the group consisting of.
(1) nucleoside reverse transcriptase translocation inhibitors (“NRTTIs”), such as 4′-Ethynyl-2-fluoro-2′-deoxyadenosine triphosphate (also known as MK-8591 and EFdA);
(2) nucleoside or nucleotide reverse transcriptase inhibitors (“NRTIs”), such as tenofovir alafenamide fumarate, tenofovir alafenamide, tenofovir alafenamide hemifumarate, GS-9131, and GS-9148;
(3) non-nucleoside or non-nucleotide reverse transcriptase inhibitors (“NNRTIs”), such as efavirenz, etravirine, rilpivirine, nevirapine, and delavirdine;
(4) protease Inhibitors (“PIs”), such as amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, and TMC-31091;
(5) integrase strand transfer inhibitors (“INSTIs”), such as Bictegravir, cabotegravir, raltegravir, and dolutegravir.

In a particular embodiment, a crystalline form, an amorphous form, a salt, or a co-crystal of the compound of Formula I disclosed herein, is provided for use to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP). Accordingly, in certain embodiments, methods for reducing the risk of acquiring HIV (e.g., HIV-1 and/or HIV-2) are provided. For example, methods for reducing the risk of acquiring HIV (e.g., HIV-1 and/or HIV-2) comprise administration of a crystalline form, an amorphous form, a salt, or a co-crystal of the compound of Formula I disclosed herein. In certain embodiments, methods for reducing the risk of acquiring HIV (e.g., HIV-1 and/or HIV-2) comprise administration of a crystalline form, an amorphous form, a salt, or a co-crystal of the compound of Formula I disclosed herein, in combination with one or more additional therapeutic agents. In certain embodiments, methods for reducing the risk of acquiring HIV (e.g., HIV-1 and/or HIV-2) comprise administration of a crystalline form, an amorphous form, a salt, or a co-crystal of the compound of Formula I disclosed herein, and a pharmaceutically acceptable excipient.

In some embodiments, a crystalline form, an amorphous form, a salt, or a co-crystal of the compound of Formula I disclosed herein, is provided for use to prevent HIV infection from taking hold if the individual is exposed to the virus and/or to keep the virus from establishing a permanent infection and/or to prevent the appearance of symptoms of the disease and/or to prevent the virus from reaching detectable levels in the blood, for example for pre-exposure prophylaxis (PrEP) or post-exposure prophylaxis (PEP). Accordingly, in certain embodiments, methods for reducing the risk of acquiring HIV (e.g., HIV-1 and/or HIV-2) are provided. For example, methods for reducing the risk of acquiring HIV (e.g., HIV-1 and/or HIV-2) comprise administration of a crystalline form, an amorphous form, a salt, or a co-crystal of the compound of Formula I disclosed herein. In certain embodiments, methods for reducing the risk of acquiring HIV (e.g., HIV-1 and/or HIV-2) comprise administration of a crystalline form, an amorphous form, a salt, or a co-crystal of the compound of Formula I disclosed herein, in combination with one or more additional therapeutic agents. In certain embodiments, methods for reducing the risk of acquiring HIV (e.g., HIV-1 and/or HIV-2) comprise administration of a crystalline form, an amorphous form, a salt, or a co-crystal of the compound of Formula I disclosed herein, and a pharmaceutically acceptable excipient.

In certain embodiments, methods for reducing the risk of acquiring HIV (e.g., HIV-1 and/or HIV-2) comprise administration of a crystalline form, an amorphous form, a salt, or a co-crystal of the compound of Formula I disclosed herein, in combination with safer sex practices. In certain embodiments, methods for reducing the risk of acquiring HIV (e.g., HIV-1 and/or HIV-2) comprise administration to an individual at risk of acquiring HIV. Examples of individuals at high risk for acquiring HIV include, without limitation, an individual who is at risk of sexual transmission of HIV.

In some embodiments, methods for reducing the risk of acquiring HIV (e.g., HIV-1 and/or HIV-2) comprise administration of a crystalline form, an amorphous form, a salt, or a co-crystal of the compound of Formula I disclosed herein, in combination with safer sex practices. In certain embodiments, methods for reducing the risk of acquiring HIV (e.g., HIV-1 and/or HIV-2) comprise administration to an individual at risk of acquiring HIV. Examples of individuals at high risk for acquiring HIV include, without limitation, an individual who is at risk of sexual transmission of HIV.

In certain embodiments, the reduction in risk of acquiring HIV is at least about 40%, 50%, 60%, 70%, 80%, 90%, or 95%. In certain embodiments, the reduction in risk of acquiring HIV is at least about 75%. In certain embodiments, the reduction in risk of acquiring HIV is about 80%, 85%, or 90%.

In another embodiment, the use of a crystalline form, an amorphous form, a salt, or a co-crystal of the compound of Formula I disclosed herein, for the manufacture of a medicament for the treatment of an HIV infection in a human being having or at risk of having the infection is disclosed.

In some embodiments, the use of a crystalline form, an amorphous form, a salt, or a co-crystal of the compound of Formula I disclosed herein, for the manufacture of a medicament for the treatment of an HIV infection in a human being having or at risk of having the infection is disclosed.

Also disclosed herein is a crystalline form, an amorphous form, a salt, or a co-crystal of the compound of Formula I disclosed herein, for use in the therapeutic treatment or delaying the onset of AIDS.

In some embodiments, disclosed herein is a crystalline form, an amorphous form, a salt, or a co-crystal of the compound of Formula I disclosed herein, for use in the therapeutic treatment or delaying the onset of AIDS.

Also disclosed herein is a crystalline form, an amorphous form, a salt, or a co-crystal of the compound of Formula I disclosed herein, for use in the prophylactic or therapeutic treatment of an HIV infection.

In some embodiments, disclosed herein is a crystalline form, an amorphous form, a salt, or a co-crystal of the compound of Formula I disclosed herein, for use in the prophylactic or therapeutic treatment of an HIV infection.

In certain embodiments, a crystalline form, an amorphous form, a salt, or a co-crystal of the compound of Formula I disclosed herein, can be used as a research tool.

### Combination Therapy

In certain embodiments, a method for treating an HIV infection is provided, comprising administering to the human a therapeutically effective amount of a crystalline form, amorphous form, salt or co-crystal disclosed herein, in combination with a therapeutically effective amount of one, two, three, or four additional therapeutic agents. In one embodiment, a method for treating an HIV infection is provided, comprising administering to the human a therapeutically effective amount of a crystalline form, amorphous form, salt or co-crystal disclosed herein, or a pharmaceutically acceptable salt thereof, in combination with a therapeutically effective amount of one, two, three, or four additional therapeutic agents.

In one embodiment, pharmaceutical compositions comprising a crystalline form, amorphous form, salt or co-crystal disclosed herein, in combination with one, two, three, or four additional therapeutic agents, and a pharmaceutically acceptable carrier, diluent, or excipient are provided.

In certain embodiments, the present disclosure provides a method for treating an HIV infection, comprising administering to a subject in need thereof a therapeutically effective amount of a crystalline form, amorphous form, salt or co-crystal disclosed herein, in combination with a therapeutically effective amount of one, two, three, or four additional therapeutic agents which are suitable for treating an HIV infection.

In certain embodiments, a crystalline form, amorphous form, salt or co-crystal disclosed herein, is combined with one, two, three, four, or more additional therapeutic agents. In certain embodiments, a crystalline form, amorphous form, salt or co-crystal disclosed herein, is combined with one, two, three, or four additional therapeutic agents. In certain embodiments, a crystalline form, amorphous form, salt or co-crystal disclosed herein, is combined with two additional therapeutic agents. In other embodiments, a crystalline form, amorphous form, salt or co-crystal disclosed herein, is combined with three additional therapeutic agents. In further embodiments, a crystalline form, amorphous form, salt or co-crystal disclosed herein, is combined with four additional therapeutic agents. The one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a crystalline form, amorphous form, salt or co-crystal disclosed herein is administered with one, two, three, or four additional therapeutic agents. Co-administration of a crystalline form, amorphous form, salt or co-crystal disclosed herein disclosed herein with one, two, three, or four additional therapeutic agents generally refers to simultaneous or sequential administration of a crystalline form, amorphous form, salt or co-crystal disclosed herein and one, two, three, or four additional therapeutic agents, such that therapeutically effective amounts of the crystalline form, amorphous form, salt or co-crystal disclosed herein and the one, two, three, or four additional therapeutic agents are both present in the body of the patient. When administered sequentially, the combination may be administered in two or more administrations.

Co-administration includes administration of unit dosages a crystalline form, amorphous form, salt or co-crystal disclosed herein before or after administration of unit dosages of one, two, three, or four additional therapeutic agents. For example, a crystalline form, amorphous form, salt or co-crystal disclosed herein may be administered within seconds, minutes, or hours of the administration of the one, two, three, or four additional therapeutic agents. In some embodiments, a unit dose of a crystalline form, amorphous form, salt or co-crystal disclosed herein is administered first, followed within seconds or minutes by administration of a unit dose of one, two, three, or four additional therapeutic agents. Alternatively, a unit dose of one, two, three, or four additional therapeutic agents is administered first, followed by administration of a unit dose of a crystalline form, amorphous form, salt or co-crystal disclosed herein within seconds or minutes. In other embodiments, a unit dose of a crystalline form, amorphous form, salt or co-crystal disclosed herein is administered first, followed, after a period of hours (e.g., 1-12 hours), by administration of a unit dose of one, two, three, or four additional therapeutic agents. In yet other embodiments, a unit dose of one, two, three, or four additional therapeutic agents is administered first, followed, after a period of hours (e.g., 1-12 hours), by administration of a unit dose of a compound disclosed herein.

In certain embodiments, a kit comprising a crystalline form, amorphous form, salt or co-crystal disclosed herein, in combination with one or more (e.g., one, two, three, or four) additional therapeutic agents is provided.

In a specific embodiment, the kit includes a crystalline form, amorphous form, salt or co-crystal disclosed herein, an HIV nucleoside or nucleotide inhibitor of reverse transcriptase and an HIV capsid inhibitor or an HIV capsid polymerization inhibitor.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drug products for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drug products for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from dolutegravir, cabotegravir, islatravir, darunavir, bictegravir, elsulfavirine, rilpivirine, and lenacapavir, and combinations thereof. In some embodiments, the additional therapeutic agent or agents is lenacapavir.

### HIV Combination Drug Products

Examples of combination drug products include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir), NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911.

Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), doravirine+islatravir (fixed dose combination/oral tablet formulation, HIV-1 infection), elsulfavirine (long acting injectable nanosuspension, HIV infection), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, islatravir, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV non-catalytic site, or allosteric, integrase inhibitors (NCINI) include, but are not limited to, those described in U.S. Patent Application Publication Nos. US2014221356 and US2016016973.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, Cl3hmAb, lipuvirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HIBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CA1, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript IE (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In various embodiments, the crystalline forms, amorphous forms, salts and co-crystals as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRiKE) include, but are not limited to, OXS-3550, HIV-TriKE, and CD16-IL-15-B7H3 TriKe.

Indoleamine-pyrrole-2,3-dioxygenase (IDO1) Inhibitors

In various embodiments, the crystalline forms, amorphous forms, salts and co-crystals as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the crystalline forms, amorphous forms, salts and co-crystals as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, 5-540956, litenimod, MGN-1601, BB-001, B1-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the crystalline forms, amorphous forms, salts and co-crystals described herein are combined with an inhibitor or antagonist of CDK. In some embodiments, the CDK inhibitor or antagonist is selected from the group consisting of VS2-370.

### Sting Agonists, RIG-I and NOD2 Modulators

In some embodiments, the crystalline forms, amorphous forms, salts and co-crystals described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the crystalline forms, amorphous forms, salts, and co-crystals as described herein are combined with an anti-TIM-3 antibody, such as TSR-022, LY-3321367, MBG-453, INCAGN-2390.

In certain embodiments, the crystalline forms, amorphous forms, salts, and co-crystals herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the crystalline forms, amorphous forms, salts and co-crystals described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated Il-15), P-22339, and a IL-15-PD-1 fusion protein N-809; examples of IL-7 include without limitation CYT-107.

Examples of additional immune-based therapies that can be combined with the crystalline forms, amorphous forms, salts, and co-crystals of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acids Res., 42 (Database issue):Dl 133-9 (2014), Mascola et al., Immunol Rev., 254(1):225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue):Dl 133-9 (2014), Mascola et al., Immunol Rev., 254(1):225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, D1D2, 10-1074-LS, Cl3hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the crystalline forms, amorphous forms, salts and co-crystals disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, the crystalline forms, amorphous forms, salts and co-crystals disclosed herein is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, the crystalline forms, amorphous forms, salts and co-crystals disclosed herein are combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the crystalline forms, amorphous forms, salts and co-crystals described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupT1 cell-based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the crystalline forms, amorphous forms, salts and co-crystals disclosed herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the crystalline forms, amorphous forms, salts, and co-crystals disclosed herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the crystalline forms, amorphous forms, salts, and co-crystals disclosed herein are combined with a population of TCR-T-cells. TCR-T-cells are engineered to target HIV derived peptides present on the surface of virus-infected cells, for example, ImmTAV.

### B-Cell Therapy

In certain embodiments, the crystalline forms, amorphous forms, salts, and co-crystals disclosed herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

The crystalline form, amorphous form, salt or co-crystal disclosed herein may be combined with one, two, three, or four additional therapeutic agents in any dosage amount of the crystalline form, amorphous form, salt or co-crystal (e.g., from 1 mg to 1000 mg of compound).

In one embodiment, kits comprising crystalline form, amorphous form, salt, or co-crystal disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MILK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drug products for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long-acting regimens include, but are not limited to, cabotegravir, rilpivirine, any integrase LA, VM-1500 LAI, maraviroc (LAI), tenofovir implant, islatravir implant, doravirine, raltegravir, and long acting dolutegravir.

### Routes of Administration

The crystalline forms, amorphous forms, salts, and co-crystals disclosed herein can be administered by any route appropriate to the condition to be treated. Suitable routes include oral, rectal, nasal, topical (including buccal and sublingual), transdermal, vaginal, and parenteral (including subcutaneous, intramuscular, intravenous, intradermal, intrathecal and epidural), and the like. It will be appreciated that the preferred route may vary with, for example, the condition of the recipient. In certain embodiments, the crystalline forms, amorphous forms, salts and co-crystals disclosed herein can be dosed parenterally. In certain embodiments, crystalline forms, amorphous forms, salts, and co-crystals disclosed herein can be dosed intravenous, subcutaneous, or intramuscular. In certain embodiments, the crystalline forms, amorphous forms, salts, and co-crystals disclosed herein are dosed orally. In certain embodiments, the crystalline forms, amorphous forms, salts, and co-crystals disclosed herein are dosed subcutaneously. In certain embodiments, the crystalline forms, amorphous forms, salts, and co-crystals disclosed herein are dosed intramuscularly.

In some embodiments, the crystalline forms, amorphous forms, salts, and co-crystals disclosed herein may be administered with a syringe suitable for administration of the compound. In some embodiments, the syringe is disposable. In some embodiments, the syringe is reusable. In some embodiments, the syringe is pre-filled with a crystalline form, amorphous form, salt and co-crystal disclosed herein.

In some embodiments, the crystalline forms, amorphous forms, salts, and co-crystals disclosed herein, may be administered via injection, using an injection device. In some embodiments, the injection device is or includes a syringe, which can be employed manually, or as part of a syringe-containing injection device, such as, but not limited to, one with a needle safety shield. A wide variety of injection devices can be used, such as, for example and not limited to, a handheld or wearable autoinjector, a handheld or wearable manual injector, an on-body injector, a syrette, a jet injector, or a pen injector, each of which can be reusable or disposable.

In some embodiments, the crystalline forms, amorphous forms, salts, and co-crystals disclosed herein may be administered with an auto-injector comprising a syringe. In some embodiments, the syringe is disposable. In some embodiments, the syringe is reusable. In some embodiments, the syringe is pre-filled with a crystalline form, amorphous form, salt, or co-crystal disclosed herein.

### Dosing Regimen

The crystalline forms, amorphous forms, salts, and co-crystals disclosed herein, may be administered to a subject in accordance with an effective dosing regimen for a desired period of time or duration, such as at least about one day, at least about one week, at least about one month, at least about 2 months, at least about 3 months, at least about 4 months, at least about 6 months, or at least about 12 months or longer. In one variation, the crystalline forms, amorphous forms, salts, and co-crystals disclosed herein are administered on a daily or intermittent schedule. In one variation, the crystalline forms, amorphous forms, salts, and co-crystals disclosed herein are administered on a monthly schedule. In one variation, the crystalline forms, amorphous forms, salts, and co-crystals disclosed herein are administered every two months. In one variation, the crystalline forms, amorphous forms, salts, and co-crystals disclosed herein are administered every three months. In one variation, the crystalline forms, amorphous forms, salts, and co-crystals disclosed herein are administered every four months. In one variation, the crystalline forms, amorphous forms, salts, and co-crystals disclosed herein are administered every five months. In one variation, the crystalline forms, amorphous forms, salts, and co-crystals disclosed herein are administered every 6 months.

In some embodiments, the crystalline forms, amorphous forms, salts, and co-crystals disclosed herein may be administered to a subject at least about one month, at least about two months, at least about 3 months, at least about 4 months, or at least about 6 months. In some embodiments, the crystalline forms, amorphous forms, salts, and co-crystals disclosed herein may be subcutaneously administered to a subject at least about one month. In some embodiments, the crystalline forms, amorphous forms, salts, and co-crystals disclosed herein may be subcutaneously administered to a subject at least about two months. In some embodiments, the crystalline forms, amorphous forms, salts and co-crystals disclosed herein may be subcutaneously administered to a subject at least about three months.

In some embodiments, the crystalline forms, amorphous forms, salts, and co-crystals disclosed herein may be intramuscularly administered to a subject at least about one month. In some embodiments, the crystalline forms, amorphous forms, salts, and co-crystals disclosed herein may be intramuscularly administered to a subject at least about two months. In some embodiments, the crystalline forms, amorphous forms, salts and co-crystals disclosed herein may be intramuscularly administered to a subject at least about three months.

In some embodiments, the crystalline forms, amorphous forms, salts, and co-crystals disclosed herein may be subcutaneously or intramuscularly administered to a subject at least about 3 months, at least about 4 months, at least about 5 months, or at least about 6 months.

In some embodiments, the crystalline forms, amorphous forms, salts, and co-crystals disclosed herein may be orally administered to a subject. In some embodiments, the crystalline forms, amorphous forms, salts, and co-crystals disclosed herein may be orally administered as an immediate release formulation.

The dosage or dosing frequency of the crystalline forms, amorphous forms, salts, and co-crystals disclosed herein may be adjusted over the course of the treatment, based on the judgment of the administering physician.

The crystalline forms, amorphous forms, salts, and co-crystals disclosed herein may be administered to a subject (e.g., a human) in an effective amount. In certain embodiments, the crystalline forms, amorphous forms, salts, and co-crystals disclosed herein are administered once daily. In certain embodiments, the crystalline forms, amorphous forms, salts, and co-crystals disclosed herein are administered once weekly. In certain embodiments, the crystalline forms, amorphous forms, salts, and co-crystals disclosed herein are administered once monthly. In certain embodiments, the crystalline forms, amorphous forms, salts, and co-crystals disclosed herein are administered once every three months.

The crystalline forms, salts, and co-crystals disclosed herein may be administered in a dosage amount that is effective. For example, the dosage amount can be from 1 mg to 3000 mg, from 1 mg to 2400 mg, or from 1 mg to 1000 mg of compound. In certain embodiments, the dosage amount is about 1, 10, 20, 30, 40, 50, 60, 70, 80, 90, 95, 100, 105, 110, 120, 130, 140, or 150 mg of the crystalline form, amorphous form, salt, or co-crystal. In certain embodiments the dosage amount is about 100, 150, 200, 250, 300, 350, 400, 450, 500, 550, 600, 650, 700, 750, 800, 850, 900, 950, or 1000 mg. In certain embodiments, the dosage amount is about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 mg.

In some embodiments, the crystalline forms, amorphous forms, salts, and co-crystals disclosed herein are administered monthly. In some embodiments, the crystalline forms, amorphous forms, salts, and co-crystals disclosed herein are administered monthly at a dose of about 150-250 mg. In some embodiments, the crystalline forms, amorphous forms, salts, and co-crystals disclosed herein are administered monthly at a dose of about 200-250 mg.

In some embodiments, the crystalline forms, amorphous forms, salts, and co-crystals disclosed herein are administered once every two months. In some embodiments, the crystalline forms, amorphous forms, salts, and co-crystals disclosed herein are administered once every two months at a dose of about 400-650 mg. In some embodiments, the crystalline forms, amorphous forms, salts, and co-crystals disclosed herein are administered once every two months at a dose of about 450-600 mg.

In some embodiments, the crystalline forms, amorphous forms, salts, and co-crystals disclosed herein are administered once every three months. In some embodiments, the crystalline forms, amorphous forms, salts, and co-crystals disclosed herein are administered once every three months at a dose of about 800-1300 mg.

In some embodiments, the crystalline forms, amorphous forms, salts, and co-crystals disclosed herein are administered weekly. In some embodiments, the crystalline forms, amorphous forms, salts, and co-crystals disclosed herein are administered weekly at a dose of about 150-250 mg. In some embodiments, the crystalline forms, amorphous forms, salts, and co-crystals disclosed herein are administered weekly at a dose of about 200-250 mg. In some embodiments, the crystalline forms, amorphous forms, salts, and co-crystals disclosed herein are administered weekly at a dose of about 400-650 mg. In some embodiments, the crystalline forms, amorphous forms, salts, and co-crystals disclosed herein are administered weekly at a dose of about 450-600 mg. In some embodiments, the crystalline forms, amorphous forms, salts, and co-crystals disclosed herein are administered weekly at a dose of about 800-1300 mg.

### Pharmaceutical Compositions

Pharmaceutical compositions disclosed herein comprise a crystalline form, amorphous form, salt, or co-crystal disclosed herein together with one or more pharmaceutically acceptable excipients and optionally other therapeutic agents. Pharmaceutical compositions containing the crystalline form, amorphous form, salt, or co-crystal disclosed herein may be in any form suitable for the intended method of administration.

Pharmaceutical compositions comprising the crystalline form, amorphous form, salt, or co-crystal disclosed herein may be prepared with conventional carriers (e.g., inactive ingredient or excipient material) which may be selected in accord with ordinary practice. Tablets may contain excipients including glidants, fillers, binders and the like. Aqueous compositions may be prepared in sterile form, and when intended for delivery by other than oral administration generally may be isotonic. All compositions may optionally contain excipients such as those set forth in the Rowe et al, Handbook of Pharmaceutical Excipients, 5^{th} edition, American Pharmacists Association, 1986. Excipients can include ascorbic acid and other antioxidants, chelating agents such as EDTA, carbohydrates such as dextrin, hydroxyalkylcellulose, hydroxyalkylmethylcellulose, stearic acid and the like.

While it is possible for the active ingredient (e.g., the crystalline form, amorphous form, salt, or co-crystal disclosed herein) to be administered alone, it may be preferable to present the active ingredient as pharmaceutical compositions. The compositions, both for veterinary and for human use, comprise at least the crystalline form, amorphous form, salt, or co-crystal disclosed herein together with one or more acceptable carriers and optionally other therapeutic ingredients. In one embodiment, the pharmaceutical composition comprises a crystalline form, amorphous form, salt, or co-crystal disclosed herein, a pharmaceutically acceptable excipient, and a therapeutically effective amount of one or more (e.g., one, two, three, or four; or one or two; or one to three; or one to four) additional therapeutic agents as defined hereinbefore. In one embodiment, the pharmaceutical composition comprises a crystalline form, amorphous form, salt, or co-crystal disclosed herein, a pharmaceutically acceptable excipient, and one other therapeutic ingredient. The carrier(s) are “acceptable” in the sense of being compatible with the other ingredients of the composition and physiologically innocuous to the recipient thereof.

The compositions include those suitable for various administration routes. The compositions may conveniently be presented in unit dosage form and may be prepared by any of the methods well known in the art of pharmacy. Such methods include the step of bringing into association the active ingredient (e.g., the crystalline form, amorphous form, salt, or co-crystal disclosed herein) with one or more inactive ingredients (e.g., a carrier, pharmaceutical excipient, etc.). The compositions may be prepared by uniformly and intimately bringing into association the active ingredient with liquid carriers or finely divided solid carriers or both, and then, if necessary, shaping the product. Techniques and formulations generally are found in Remington: The Science and Practice of Pharmacy, 21^{st} Edition, Lippincott, Wiliams and Wilkins, Philadelphia, Pa., 2006.

Compositions described herein that are suitable for oral administration may be presented as discrete units (a unit dosage form) including but not limited to capsules, cachets, or tablets each containing a predetermined amount of the active ingredient. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents, and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium carbonate or sodium carbonate, lactose, lactose monohydrate, croscarmellose sodium, povidone, calcium phosphate or sodium phosphate; granulating and disintegrating agents, such as maize starch, or alginic acid; binding agents, such as cellulose, microcrystalline cellulose, starch, gelatin, or acacia; lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, disclosed herein are oral dosage forms (e.g., tablets), which may be prepared from hot melt extrusion or spray-drying dispersion (SDD) technologies.

In some embodiments, disclosed herein are hard capsules filled with powder, beads, or granules containing the active ingredient in admixture with non-toxic pharmaceutically acceptable excipient which are suitable for manufacture of hard or soft capsules. These excipients may be, for example, inert diluents, such as calcium carbonate or sodium carbonate, lactose, lactose monohydrate, croscarmellose sodium, povidone, calcium phosphate or sodium phosphate; granulating and disintegrating agents, such as maize starch, or alginic acid; binding agents, such as cellulose, microcrystalline cellulose, starch, gelatin, or acacia; and lubricating agents, such as magnesium stearate, stearic acid or talc.

In some embodiments, disclosed herein are hard or soft capsules filled with liquid or semi-solid mixtures containing the active ingredient in admixture with non-toxic pharmaceutically acceptable excipient which are suitable for manufacture of hard or soft capsules. These excipients may be, for example, solubilizing oils such as maize oil, sesame oil, or corn oil; medium chain triglycerides and related esters, such as, derivatized palm kernel oil or coconut oil; self-emulsifying lipid systems (SEDDS or SMEDDS), such as caprylic triglyceride or propylene glycol monocaprylate; viscosity modifiers, such as, cetyl alcohol, steryl alcohol, glycerol stearate; and solubilizing agents and surfactants, such as polyethylene glycol, propylene glycol, glycerin, ethanol, polyethoxylated castor oil, poloxamers, or polysorbates.

The pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the sterile injectable preparation disclosed herein may also be a sterile injectable solution or suspension prepared from a reconstituted lyophilized powder in a non-toxic parenterally acceptable diluent or solvent, such as a solution in 1,3-butane-diol. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. In certain embodiments the suspension is a microsuspension. In certain embodiments the suspension is a nanosuspension.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are known to the person skilled in the art of parenteral formulation and may be found e.g., in Handbook of Pharmaceutical Excipients (eds. Rowe, Sheskey & Quinn), 6th edition 2009.

Examples of solubilizing excipients in a parenteral formulation (e.g., an SC or IM formulation) include, but are not limited to, polyethylene glycol (PEG), polysorbates (such as polysorbate 20 or 80) and poloxamers (such as poloxamer 338, 188, or 207).

In some embodiments, the parenteral formulation (e.g., an SC or IM formulation) disclosed herein is an aqueous formulation. In some embodiments, the parenteral formulation (e.g., an SC or IM formulation) disclosed herein is an aqueous suspension that comprises a crystalline form, amorphous form, salt, or a co-crystal disclosed herein and water.

In certain embodiments, the composition is disclosed as a solid dosage form, including a solid injectable dosage form, such as a solid depot form.

The amount of active ingredient that may be combined with the inactive ingredients to produce a dosage form may vary depending upon the intended treatment subject and the particular mode of administration. For example, in some embodiments, a dosage form for oral or parenteral administration to humans may contain approximately 1 to 2400 mg of active material formulated with an appropriate and convenient amount of carrier material (e.g., inactive ingredient or excipient material). In certain embodiments, the carrier material varies from about 5 to about 95% of the total compositions (weight:weight).

It should be understood that in addition to the ingredients particularly mentioned above the compositions of these embodiments may include other agents conventional in the art having regard to the type of composition in question, for example, those suitable for oral administration may include flavoring agents.

In certain embodiments, a composition comprising an active ingredient disclosed herein in one variation does not contain an agent that affects the rate at which the active ingredient is metabolized. Thus, it is understood that compositions comprising a crystalline form, amorphous form, salt, or co-crystal disclosed herein, in certain embodiments do not comprise an agent that would affect (e.g., slow, hinder or retard) the metabolism of the crystalline form, amorphous form, salt, or co-crystal, or any other active ingredient administered separately, sequentially or simultaneously with the salt, co-crystal, or crystalline form. It is also understood that any of the methods, kits, articles of manufacture, and the like detailed herein do not comprise an agent that would affect (e.g., slow, hinder or retard) the metabolism of a crystalline form, amorphous form, salt, or co-crystal, or any other active ingredient administered separately, sequentially or simultaneously with the crystalline form, amorphous form, salt, or co-crystal.

### Kits and Articles of Manufacture

The present disclosure relates to a kit comprising a crystalline form, amorphous form, salt, or co-crystal disclosed herein. In one embodiment, the kit may comprise one or more additional therapeutic agents as described herein before. The kit may further comprise instructions for use, e.g., for use in inhibiting an HIV reverse transcriptase, such as for use in treating an HIV infection or AIDS or as a research tool. The instructions for use are generally written instructions, although electronic storage media (e.g., magnetic diskette or optical disk) containing instructions are also acceptable.

The present disclosure also relates to a pharmaceutical kit comprising one or more containers comprising crystalline form, amorphous form, salt, or co-crystal disclosed herein. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

In some embodiments, the present disclosure also relates to a pharmaceutical kit comprising one or more containers comprising a crystalline form, amorphous form, salt, or co-crystal disclosed herein. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

Also disclosed are articles of manufacture comprising a unit dosage of a crystalline form, amorphous form, salt, or co-crystal, disclosed herein in suitable packaging for use in the methods described herein. Suitable packaging is known in the art and includes, for example, vials, vessels, ampules, bottles, jars, flexible packaging, prefilled syringes, and the like. An article of manufacture may further be sterilized and/or sealed.

### EXAMPLES

### General Methods

XRPD patterns were collected with a PANalytical Empyrean diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning line detector, PIXcel1D-Medipix3 PASS (programmable anti-scatter slit), located 240 mm from the sample and Data Collector software v. 7.2b.

DSC was run on a Q2000 (TA Instruments, New Castle, DE) by loading 1-10 mg of material into a crimped or open Tzero standard aluminum pan and heating the sample at 10° C./min from 20 to 300° C. or above. The sample and reference pans were under a 50 mL/min nitrogen purge. Data analysis was completed using Universal Analysis 2000 Version 4.5A (TA Instruments, New Castle, DE).

TGA was used to evaluate sample weight loss as a function of temperature on either a Q5000 or Q500 (TA Instruments, New Castle, DE), by loading 1-10 mg of material onto a weigh pan and heating the sample to 350° C. or above at a rate of 10° C./min. The sample and reference pans were under a 60 mL/min and 40 mL/min nitrogen purge, respectively. Data analysis was completed using Universal Analysis 2000 Version 4.5A (TA Instruments, New Castle, DE).

Hygroscopicity was studied using dynamic vapor sorption (DVS, TA Q5000 SA, TA Instruments, New Castle, DE or DVS, DVS Intrinsic, Surface Measurement Systems, London, UK). A sample (1-20 mg) was placed in an aluminum DVS pan and loaded on the sample side of the twin pan balance. The water sorption and desorption were studied as a function of relative humidity (RH) at 25° C. In 10% RH increments, the relative humidity was increased from 5% RH to 95% RH and then decreased back to 5% or starting at 10% RH and increasing to 90% RH and then back down to 10% RH. Each relative humidity increment had an equilibration time of 180 minutes, unless weight change % was less than 0.002% in 30 minutes. Data analysis was performed using Universal Analysis 2000 Version 4.7A (TA Instruments, New Castle, DE) for TA DVS runs and Microsoft Excel for SMS DVS runs.

### Example 1. Compound of Formula I, Form I

Compound A was dissolved in NMP with LiCl. Reaction was heated to 100 C for about 3 hours. The reaction was cooled to room temperature and about 80 mL of 0.5M HCl was added dropwise. About 20 mL water was added and the reaction was stirred for about 1 hour. EtOAc was used to extract the product, followed by the addition of NaCl to saturate the aq. layer and another extraction with EtOAc. The EtOAc layers were combined with brine, dried over sodium sulfate, filtered, and concentrated under vacuum. Silica gel column chromatography was used to purify the product. Fractions were combined and recrystallized from EtOAc/Hexanes. The product was dissolved in EtOAc and decoloring carbon was added. This was stirred for 30 minutes, then filtered and washed with EtOAc and MeOH. The solution was concentrated under vacuum and solid material was obtained of Formula I.

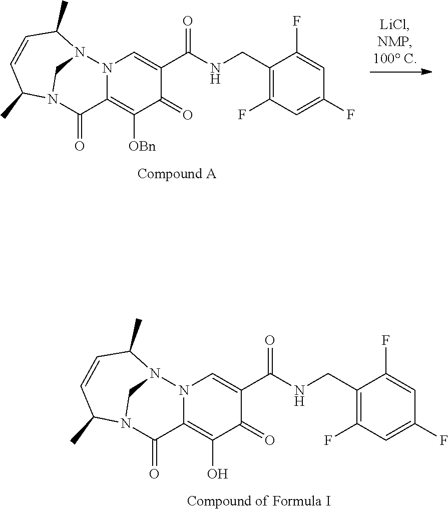

A solution of Compound B in ethyl acetate was charged to a reactor. N-methyl-2-pyrolidinone (3 V) was charged to the solution. The solution was then distilled to about 4V. LiCl (3 equiv.) was then charged before adjusting the temperature to about 80° C. for about 4 hours. The reaction was then cooled to about 20° C. before washing the organic layer with HCl (0.5M, 5 V), NaCl (5%, 10 V), and water (5 V). The organic layer was then distilled to about 6 V before charging 2-propanol (10 V). The solution was then distilled to about 6 V before charging more 2-propanol (2 V). The temperature was then adjusted to about 60° C. for about 1 hour before adjusting to about 20° C. over about 2 hours. The slurry was aged for about 1 hour to overnight before putting the slurry through a high shear wet mill for about 1 hour. The slurry was then filtered and the cake was rinsed with isopropanol before drying at about 40° C.

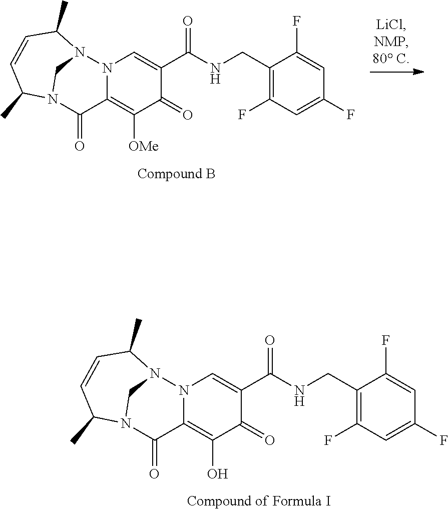

The Compound of Formula I, Form I XRPD pattern is shown in FIG. 1 and is characterized by Tier 1 reflections at 7.0, 27.9, 13.9° 2θ, but also Tier 2 at 12.3, 24.6, 17.4° 2θ, and Tier 3 29.2, 23.2, 21.4° 2θ. A list of 2-theta peaks is provided below:

Peak Table

| Pos. | Rel. Int. |
|---|---|
| [°2Th.] | [%] |
| 7.0 | 100 |
| 12.3 | 44 |
| 13.9 | 57 |
| 15.9 | 9 |
| 16.7 | 10 |
| 17.1 | 34 |
| 17.4 | 40 |
| 20.1 | 8 |
| 21.4 | 12 |
| 22.6 | 4 |
| 23.2 | 18 |
| 24.6 | 41 |
| 25.6 | 7 |
| 26.3 | 5 |
| 26.7 | 7 |
| 27.8 | 42 |
| 27.9 | 76 |
| 29.2 | 20 |
| 31.2 | 4 |
| 35.0 | 12 |
| 36.6 | 2 |
| 37.4 | 2 |

The DSC curve is shown in FIG. 2 and displays one endothermic transition at about 192° C. The TGA curve is shown in FIG. 3 and indicates that the phase is unsolvated. The DVS curve is shown in FIG. 4 and the data indicate that the form absorbs about 0.15% of water up to 95% RH at 25° C. The material was found to not have changed form post experiment.

Single crystal data was collected on the compound of Formula I, Form I and the data are summarized in Table 1 and FIG. 5.

The crystal system is monoclinic and the space group is P21. The cell parameters and calculated volume are: a=8.9146(2) Å, b=8.6717(2) Å, c=12.7101(3) Å, α=90°, β=93.3170(10°), γ=90°, V=980.95(4) Å³. The molecular weight is 448.40 g mol⁻¹ with Z=2, resulting in a calculated density of 1.518 g cm⁻³.

### Example 2. Compound of Formula I, Form II

Compound of Formula I, Form II was isolated when excess Compound of Formula I, Form I was added to about 0.5 mL THE to create a slurry, then the slurry was stirred at room temperature for about 6 days. The slurry was filtered and XRPD was taken of the wet solids. The sample was dried in a vacuum oven then further characterized.

The XRPD pattern for the compound of Formula I, Form II is shown in FIG. 6 and is characterized by Tier 1 reflections at 5.7, 19.9, 26.8° 2θ, but also Tier 2 at 28.6, 11.4, 17.6° 2θ, and Tier 3 at 22.3, 25.1, 10.7° 2θ. A list of 2-theta peaks is provided below:

Peak Table

| Pos. | Rel. Int. |
|---|---|
| [°2Th.] | [%] |
| 5.7 | 100 |
| 10.7 | 11 |
| 11.4 | 23 |
| 11.7 | 11 |
| 13.3 | 8 |
| 14.9 | 4 |
| 16.5 | 3 |
| 17.1 | 7 |
| 17.6 | 19 |
| 19.6 | 9 |
| 19.9 | 35 |
| 21.4 | 4 |
| 22.3 | 14 |
| 22.9 | 3 |
| 23.6 | 3 |
| 24.5 | 7 |
| 25.1 | 12 |
| 25.5 | 5 |
| 26.8 | 25 |
| 28.6 | 25 |
| 30.5 | 4 |
| 30.9 | 4 |
| 34.7 | 2 |

The DSC curve for the compound of Formula I, Form II is shown in FIG. 7 and displays an endothermic transition at about 194° C. The TGA curve for the compound of Formula I, Form II is shown in FIG. 8 and indicates that the phase is unsolvated.

### Example 3. Compound of Formula I, Sodium Salt, Form I

Compound of Formula I, sodium salt, Form I was isolated when about 1000 mg Compound of Formula I, Form I was dissolved in about 6 mL methanol at about 45° C. About 133 mg of sodium hydroxide was added. The sample was sonicated for about 1 hour, then left to stir at room temperature as an open vial until all the sample was evaporated and dry solids remained. The dry solids were combined with about 1 mL methanol and about 3 mL water and sonicated for about 30 minutes, then left to stir at room temperature as an open vial until all the sample was evaporated and dry solids remained. The dry solids were combined with about 1 mL methanol and about 3 mL water and sonicated for about 30 minutes, then left to stir at room temperature as an open vial until all the sample was evaporated and dry solids remained. The dry solids were reslurried in 30% methanol in water at room temperature for about 3 weeks. The slurry was filtered and dried in a vacuum oven overnight at about 50° C.

The Compound of Formula I, sodium salt, Form I XRPD pattern is shown in FIG. 9 and is characterized by Tier 1 reflections at 13.1, 26.8, 14.8° 2θ, but also Tier 2 at 28.2, 20.7, 17.8° 2θ, and Tier 3 at 6.2, 21.2, 23.3° 2θ. A list of 2-theta peaks is provided below:

Peak Table

| Pos. | Rel. Int. |
|---|---|
| [°2Th.] | [%] |
| 4.9 | 11 |
| 6.2 | 47 |
| 7.7 | 21 |
| 9.9 | 22 |
| 11.9 | 22 |
| 12.4 | 34 |
| 13.1 | 100 |
| 14.8 | 76 |
| 16.6 | 32 |
| 17.8 | 50 |
| 18.7 | 20 |
| 19.7 | 27 |
| 20.7 | 54 |
| 21.2 | 43 |
| 21.9 | 40 |
| 23.3 | 35 |
| 25.2 | 32 |
| 25.6 | 28 |
| 26.8 | 79 |
| 28.2 | 73 |
| 29.9 | 23 |
| 36.6 | 15 |

The DSC curve is shown in FIG. 10 and displays an endothermic transition at about 25° C., 81° C., and 124° C., and exothermic transition at about 151° C. The TGA curve is shown in FIG. 11 and indicates that there is weight loss starting at around ambient temperature. The DVS curve is shown in FIG. 12 and the data indicate that the form absorbs about 4% of water up to 95% RH at 25° C. The material was found to not have changed form post experiment.

### Example 4. Compound of Formula I, Sodium Salt, Form II

Compound of Formula I, sodium salt, Form II was isolated when about 1000 mg Compound of Formula I, Form I was added to a vial with about 19 mL of methanol. In a separate vial, about 1 mL water was combined with 2 mol equiv. sodium hydroxide to create a solution. The two solutions were combined, and 1 mL was withdrawn and dispensed into a 4 mL vial. A miVac system was used to remove the solvent resulting in a dry solid in the vial. About 1 mL water was added to the dry solids and stirred at room temperature overnight. The resulting solids were suction filtered and XRPD was taken of the wet solids. The solids were then dried and further characterized.

Compound of Formula I, sodium salt, Form II XRPD pattern is shown in FIG. 13 and is characterized by reflections at 5.3° 7.1°, 13.4°, and 10.7°2θ. A list of 2-theta peaks is provided below.

Peak Table

| Pos. | Rel. Int. |
|---|---|
| [°2Th.] | [%] |
| 5.3 | 100 |
| 7.1 | 44 |
| 10.7 | 15 |
| 13.4 | 41 |

The DSC curve for the Compound of Formula I, sodium salt, Form II is shown in FIG. 14 and displays an endothermic transition at about 32° C. and an endothermic transition at about 122° C. The TGA curve for the Compound of Formula I, sodium salt, Form II is shown in FIG. 15 and indicates that the phase loses mass starting at about ambient temperature.

### Example 5. Compound of Formula I, Sodium Salt, Form III

Compound of Formula I, sodium salt, Form III was isolated when about 0.1 g Compound of Formula I, Form I and about 2.5 equiv. of sodium acetate was combined with 4 volumes of isopropyl acetate and stirred with a stir bar. A solution was observed. Heptane was added dropwise until solids precipitated out of solution. The solids were filtered and characterized by XRPD.

Compound of Formula I, sodium salt, Form III XRPD pattern is shown in FIG. 16 and is characterized by Tier 1 reflections at 7.9, 8.8, 23.1° 2θ, and 26.2, 26.6, 30.3 Tier 2 at °2θ. A list of 2-theta peaks is provided below.

Peak Table

| Pos. | Rel. Int. |
|---|---|
| [°2Th.] | [%] |
| 7.9 | 18 |
| 8.8 | 100 |
| 23.1 | 17 |
| 26.2 | 9 |
| 26.6 | 7 |
| 30.3 | 5 |

### Example 6. Compound of Formula I, Sodium Salt, Form IV

Compound of Formula I, sodium salt, Form IV was combined with about 1.1 equiv sodium hydroxide, 4.35 V water, and 2.3 V methanol in a vial with a stir bar. The vial was placed on a hot plate at about 50° C. Seeds of Compound of Formula I sodium were charged and a slurry was observed. The vial was removed from heat. Solids were filtered and the filter cake was rinsed with water. Solids were dried in a vacuum oven at about 50° C. several days. XRPD data was collected.

Compound of Formula I, sodium salt, Form IV XRPD pattern is shown in FIG. 17 and is characterized by Tier 1 reflections at 5.3, 6.4, 12.8° 2θ, and Tier 2 at 14.1, 24.5, 26.4°2θ, and Tier 3 at 8.4, 15.8, 20.7°2θ. A list of 2-theta peaks is provided below.

Peak Table

| Pos. | Rel. Int. |
|---|---|
| [°2Th.] | [%] |
| 5.3 | 100 |
| 6.4 | 41 |
| 8.4 | 13 |
| 10.5 | 11 |
| 12.8 | 30 |
| 14.1 | 24 |
| 15.8 | 18 |
| 17.5 | 11 |
| 18.6 | 8 |
| 19.4 | 6 |
| 20.7 | 21 |
| 21.7 | 17 |
| 22.8 | 17 |
| 23.2 | 17 |
| 24.5 | 23 |
| 26.4 | 27 |
| 27.5 | 8 |
| 29.7 | 7 |

### Example 7. Compound of Formula I, Potassium Salt, Form I

Compound of Formula I, potassium salt, Form I was isolated when approximately 1000 mg of compound of Formula I, Form I was dissolved in about 6 mL acetone at about 45° C. About 188 mg of potassium hydroxide was added and the sample was sonicated for about 1 hour then left to stir at room temperature. The slurry was filtered and dried in a vacuum oven overnight at about 50° C.

The XRPD pattern for the Compound of Formula I, potassium salt, Form I is shown in FIG. 18 and is characterized by Tier 1 reflections at 7.1°, 20.1°, 25.8° 2θ, but also Tier 2 at 6.2°, 19.2°, 13.0° 2θ, and Tier 3 at 15.5°, 22.9°, 14.2° 2θ. A list of 2-theta peaks is provided below.

Peak Table

| Pos. | Rel. Int. |
|---|---|
| [°2Th.] | [%] |
| 6.2 | 85 |
| 7.1 | 100 |
| 13.0 | 60 |
| 14.2 | 47 |
| 15.5 | 57 |
| 16.4 | 45 |
| 18.1 | 45 |
| 19.2 | 63 |
| 20.1 | 98 |
| 21.9 | 45 |
| 22.9 | 55 |
| 23.7 | 28 |
| 25.8 | 90 |
| 28.6 | 21 |
| 32.1 | 29 |
| 33.8 | 25 |

The DSC curve for compound of Formula I, potassium salt Form I, is shown in FIG. 19 and displays an endothermic transition at about 17° C. and an endothermic transition at about 230° C. The TGA curve for compound of Formula I, potassium salt Form I, is shown in FIG. 20 and indicates that the phase loses mass in multiple stages with one stage starting at about ambient temperature and the next stage starting at about 100° C. The DVS curve for compound of Formula I, potassium salt Form I, is shown in FIG. 21 and the data indicate that the form absorbs more than about 18% of water up to 95% RH at 25° C. The material was found to not have changed form post experiment.

The Single crystal data was collected on compound of Formula I, potassium salt Form I, and the data are summarized in Table 2 and FIG. 22. The crystal system is trigonal and the space group is P3221. The cell parameters and calculated volume are: a=28.6496(4) Å, b=28.6496(4) Å, c=6.89340(10) Å, α=90°, β=90°, γ=120°, V=4900.06(15) Å³. The molecular weight is 494.49 g mol⁻¹ with Z=6, resulting in a calculated density of 1.005 g cm⁻³.

### Example 8. Compound of Formula I, Potassium Salt, Form II

Compound of Formula I, potassium salt, Form II was isolated when Compound of Formula I, Form I was placed in a vial with EtOH and a magnetic stir bar. Some potassium acetate was added and the experiment was stirred at room temperature. The solids were characterized by XRPD and TGA.

The Compound of Formula I, potassium salt Form II XRPD pattern is shown in FIG. 23 and is characterized by Tier 1 reflections at 30.7°, 6.8°, and 31.4° 2θ, but also Tier 2 at 12.5°, 32.6°, and 28.0° 2θ, and Tier 3 at 24.2°, 14.3°, and 25.7° 2θ.

The TGA curve is shown in FIG. 24 and indicates that the phase loses mass in multiple stages with one stage starting at about ambient temperature and the next stage starting at about 100° C. A list of 2-theta peaks is provided below.

Peak Table

| Pos. | Rel. Int. |
|---|---|
| [°2Th.] | [%] |
| 6.8 | 94 |
| 12.5 | 41 |
| 14.3 | 11 |
| 20.3 | 8 |
| 24.2 | 14 |
| 25.7 | 10 |
| 26.9 | 6 |
| 28.0 | 21 |
| 29.7 | 10 |
| 30.7 | 100 |
| 31.4 | 43 |
| 32.3 | 12 |
| 32.6 | 23 |
| 35.0 | 9 |

### Example 9. Compound of Formula I, Potassium Salt, Form III

Compound of Formula I, potassium salt, Form III was isolated when 8.5 g of Compound of Formula I, Form I and 2.4 mL (about 1 equiv) potassium hydroxide in water (50 m/v %) was combined with 5 V water in a bottle with a stir bar. A thin slurry was observed. After overnight stirring the slurry was thicker and an additional volume of water was added. Slurry was immobile. Slurry was sonicated for 1 hour, seeded, then continued to stir. This was repeated daily for about 8 days. About 1 mL of slurry was combined with 0.1 mL methanol, then heated to 50° C. A solution formed. The solution was removed from the heat. Seed was added. About 0.3 mL methanol was added. Solids were observed and filtered. Solids characterized by XRPD.

The Compound of Formula I, potassium salt Form III XRPD pattern is shown in FIG. 25 and is characterized by Tier 1 reflections at 5.2, 13.2, 13.7° 2θ, but also Tier 2 at 11.2, 12.4, 18.7° 2θ, and Tier 3 at 6.5 and 15.0° 2θ. A list of 2-theta peaks is provided below:

Peak Table

| Pos. | Rel. Int. |
|---|---|
| [°2Th.] | [%] |
| 5.2 | 100 |
| 6.5 | 13 |
| 11.2 | 27 |
| 12.4 | 25 |
| 13.2 | 31 |
| 13.7 | 58 |
| 15.0 | 14 |
| 18.7 | 22 |

### Example 10. Compound of Formula I, Diethylamine Salt

Compound of Formula I, diethylamine salt was isolated when about 1000 mg of Compound of Formula I, Form I was dissolved in about 6 mL of acetone at about 45° C. About 0.25 mL of diethylamine was added and the sample was sonicated for about 15 min to yield a slurry. The sample was left to stir at room temperature. The slurry was filtered and dried in a vacuum oven overnight at about 50° C.

The XRPD pattern for the compound of Formula I, diethylamine salt is shown in FIG. 26 and is characterized by Tier 1 reflections at 19.5°, 9.7°, and 20.5° 2θ, but also Tier 2 at 21.9°, 26.5°, and 27.2° 2θ, and Tier 3 at 17.1°, 6.2°, and 10.7° 2θ. A list of 2-theta peaks is provided below:

Peak Table

| Pos. | Rel. Int. |
|---|---|
| [°2Th.] | [%] |
| 6.2 | 29 |
| 9.0 | 24 |
| 9.7 | 72 |
| 10.7 | 28 |
| 11.9 | 20 |
| 12.4 | 9 |
| 13.2 | 13 |
| 15.7 | 19 |
| 17.1 | 32 |
| 17.8 | 25 |
| 18.6 | 26 |
| 19.5 | 100 |
| 20.5 | 69 |
| 21.5 | 49 |
| 21.9 | 68 |
| 23.2 | 15 |
| 24.7 | 21 |
| 26.5 | 48 |
| 27.2 | 38 |
| 30.7 | 23 |
| 32.5 | 13 |
| 35.4 | 7 |

The DSC curve for the compound of Formula I, diethylamine salt is shown in FIG. 27 and displays an endothermic transition at about 140° C. The TGA curve for the compound of Formula I, diethylamine salt is shown in FIG. 28 and indicates that the phase loses mass in multiple stages with one stage starting at about 100° C. and the next stage starting at about 175° C. The DVS curve for the compound of Formula I, diethylamine salt is shown in FIG. 29 and the data indicate that the form absorbs less than about 1% of water up to 95% RH at 25° C. The material was found to not have changed form post experiment.

### Example 11. Compound of Formula I, Ammonia Salt

Compound of Formula I, ammonia salt was isolated when about 1000 mg of the compound of Formula I, Form I was dissolved in about 6 mL acetone at 45° C. About 100 uL ammonium hydroxide was added. The sample was sonicated for about 15 minutes and then left to stir at room temperature. The slurry was filtered and dried in a vacuum oven overnight at about 50° C.

The ammonia salt XRPD pattern is shown in FIG. 30 and is characterized by Tier 1 reflections at 12.1, 6.9, 12.5°2θ, but also Tier 2 at 18.4, 21.0, 25.4°2θ, and Tier 3 at 9.2, 23.1, 30.6°2θ. A list of 2-theta peaks is provided below:

Peak Table

| Pos. | Rel. Int. |
|---|---|
| [°2Th.] | [%] |
| 6.9 | 56 |
| 9.2 | 6 |
| 12.1 | 100 |
| 12.5 | 38 |
| 15.6 | 2 |
| 17.1 | 4 |
| 18.4 | 25 |
| 21.0 | 15 |
| 23.1 | 6 |
| 25.4 | 11 |
| 26.4 | 5 |
| 30.6 | 6 |

The DSC curve is shown in FIG. 31 and displays an endothermic transition at about 121° C. and an endothermic transition at about 194° C. The TGA curve is shown in FIG. 32 and indicates that the phase loses mass starting at about 80° C. The DVS curve is shown in FIG. 33 and the data indicate that the form absorbs less than about 1% of water up to 95% RH at 25° C. The material was found to not have changed form post experiment.

### Example 12. Compound of Formula I, Calcium Salt, Form I

Compound of Formula I, calcium salt, Form I was isolated when about 1000 mg of compound of Formula I, Form I was dissolved in about 6 mL acetone at 45° C. Separately, about 200 mg of potassium hydroxide was dissolved in about 6 mL water. The two solutions were combined and then about 124 mg of calcium chloride was added to yield a precipitate. About 28 mL of water was added and the sample was sonicated for about 30 minutes then left to stir at room temperature. The slurry was filtered and dried in a vacuum oven overnight at about 50° C. The calcium salt, Form I XRPD pattern can be found in FIG. 34. The DSC curve is shown in FIG. 35 and displays a glass transition at about 130° C.

### Example 13. Compound of Formula I, Calcium Salt, Form II

Compound of Formula I, calcium salt, Form II was isolated when about 0.02 g of Compound B:

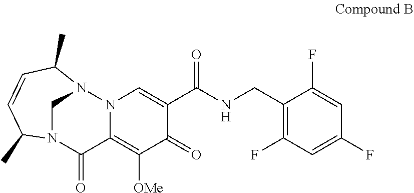

and about 0.02 g of calcium chloride was added to a vial with a stir bar and 10 V isopropanol. The vial was placed on a hot plate at 80° C. A solution was observed. Vial was removed from heat. Solids were observed and were filtered. XRPD was taken of the solids.

The calcium salt, Form II XRPD pattern is shown in FIG. 36 and is characterized by Tier 1 reflections at 7.1, 8.5, 11.7° 2θ, and Tier 2 at 14.1, 16.5, 20.1°2θ. A list of 2-theta peaks is provided below:

Peak Table

| Pos. | Rel. Int. |
|---|---|
| [°2Th.] | [%] |
| 7.1 | 100 |
| 8.5 | 7 |
| 11.7 | 5 |
| 14.1 | 6 |
| 16.5 | 7 |
| 20.1 | 7 |
| 21.5 | 5 |
| 24.8 | 5 |

### Example 14. Compound of Formula I, Calcium Salt, Form III

Compound of Formula I, calcium salt, Form III was isolated when about 1 g of Compound B:

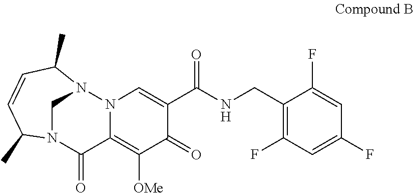

and about 0.7 g of calcium bromide was added to a vial with a stir bar and 4 V isopropanol. The vial was placed on a hot plate at 80° C. A solution was observed. After stirring for about an hour solids were observed. Vial was removed from heat. Solids were observed and were filtered. XRPD was taken of the solids.

The calcium salt, Form III XRPD pattern is shown in FIG. 37 and is characterized by Tier 1 reflections at 6.3, 7.2, 25.5° 2θ, and Tier 2 at 21.2, 28.4, 31.4° 2θ. A list of 2-theta peaks is provided below:

Peak Table

| Pos. | Rel. Int. |
|---|---|
| [°2Th.] | [%] |
| 6.3 | 100 |
| 7.2 | 35 |
| 21.2 | 11 |
| 25.5 | 22 |
| 28.4 | 11 |
| 31.4 | 9 |

### Example 15. Compound of Formula I, Magnesium Salt, Form I

Compound of Formula I, magnesium salt, Form I was isolated when about 1000 mg of compound of Formula I, Form I was dissolved in about 6 mL acetone at 45° C. Separately, about 200 mg of potassium hydroxide was dissolved in about 6 mL water. The two solutions were combined and then about 0.55 mL of 2 M magnesium chloride was added to yield a precipitate. About 28 mL of water was added and the sample was sonicated for about 30 minutes then left to stir at room temperature. The slurry was filtered and dried in a vacuum oven overnight at about 50° C.

The XRPD pattern can be found in FIG. 38. The DSC curve is shown in FIG. 39 and displays a glass transition at about 155° C.

### Example 16. Compound of Formula I, Magnesium Salt, Form II

Compound of Formula I, magnesium salt, Form II was isolated when about 1 g of compound of Compound B:

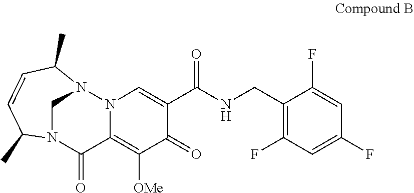

and about 1.3 g of magnesium bromide hexahydrate was added to a vial with a stir bar and 3 V isopropanol. The vial was placed on a hot plate at 90° C. Mostly a solution was observed. Solids were observed. Vial was removed from heat. Solids were observed and were filtered. XRPD was taken of the solids.

The XRPD pattern is shown in FIG. 40 is characterized by Tier 1 reflections at 7.0, 19.8, 24.3° 2θ, but also Tier 2 at 6.5, 13.0, 21.2° 2θ. A list of 2-theta peaks is provided below:

Peak Table

| Pos. | Rel. Int. |
|---|---|
| [°2Th.] | [%] |
| 6.5 | 51 |
| 7.0 | 88 |
| 13.0 | 65 |
| 19.8 | 100 |
| 21.2 | 44 |
| 24.3 | 77 |
| 25.9 | 38 |

### Example 17. Compound of Formula I, N-butylamine Salt

Compound of Formula I, N-butylamine salt was isolated when compound of Formula I, Form I was placed in a vial with EtOH and a magnetic stir bar. Some N-butylamine was added and the experiment was stirred at room temperature. The solids were characterized by XRPD. The solids were dried then further characterized.

The XRPD pattern is shown in FIG. 41 and is characterized by Tier 1 reflections at 8.0, 10.7, 5.8°2θ, but also Tier 2 at 19.2, 9.7, 11.6°2θ, and Tier 3 at 18.7, 26.5, 22.3°2θ. A list of 2-theta peaks is provided below:

Peak Table

| Pos. | Rel. Int. |
|---|---|
| [°2Th.] | [%] |
| 5.8 | 48 |
| 8.0 | 100 |
| 9.7 | 45 |
| 10.2 | 17 |
| 10.7 | 78 |
| 11.6 | 26 |
| 16.0 | 5 |
| 16.7 | 10 |
| 18.7 | 16 |
| 19.2 | 45 |
| 20.3 | 11 |
| 22.3 | 13 |
| 23.0 | 3 |
| 23.7 | 11 |
| 25.3 | 10 |
| 26.1 | 10 |
| 26.5 | 14 |
| 27.9 | 7 |
| 31.6 | 6 |

The DSC curve is shown in FIG. 42 and displays an endothermic transition at about 166° C. The TGA curve is shown in FIG. 43 and indicates that the phase loses mass in multiples stages starting at about 80° C. and another stage starting at about 175° C. The DVS curve is shown in FIG. 44 and the data indicate that the form absorbs less than about 0.2% of water up to 95% RH at 25° C. The material was found to not have changed form post experiment.

### Example 18. Compound of Formula I, Diethanolamine Salt

Compound of Formula I, diethanolamine salt was isolated when about 17.4 g of compound of Formula I, Form I was added to a 100 mL bottle with a large stir bar and about 60 mL of isopropanol and about 4.1 g diethanolamine. A solution with some solids was observed. Seed was added. After about an hour of stirring, 15 mL heptane was added along with an additional seed charge. Slurry was stirred for about 24 hours. Solids were suction filtered and the cake was rinsed with isopropanol. The solids were placed in a vacuum oven at about 40° C. Solids were characterized by XRPD.

The XRPD pattern is shown in FIG. 45 and is characterized by Tier 1 reflections at 6.1, 18.4, 19.5°2θ, but also Tier 2 at 9.9, 20.4, 21.8°2θ, and Tier 3 at 16.9, 18.9, 26.5°2θ. A list of 2-theta peaks is provided below:

Peak Table

| Pos. | Rel. Int. |
|---|---|
| [°2Th.] | [%] |
| 6.1 | 100 |
| 9.9 | 16 |
| 11.9 | 9 |
| 13.2 | 1 |
| 15.6 | 12 |
| 16.9 | 13 |
| 18.4 | 79 |
| 18.9 | 14 |
| 19.5 | 38 |
| 20.4 | 20 |
| 21.4 | 19 |
| 21.8 | 29 |
| 22.9 | 5 |
| 24.6 | 11 |
| 26.5 | 15 |
| 30.5 | 10 |
| 33.5 | 2 |

### Example 19. Compound of Formula I, Ethylenediamine Salt

Compound of Formula I, ethylenediamine salt was isolated when about 1.2 g of compound of Formula I, Form I was combined with about 66 mL methanol in a 100 mL bottle with stir bar. About 0.08 g of ethylenediamine was combined with about 3.3 mL methanol. The ethylenediamine solution was charged to the compound of Formula I, Form I solution. Immediate precipitation was observed. The solids were suction filtered and placed in a vacuum oven for about 18 hours at about 40° C. XRPD was taken of the solids.

The XRPD pattern is shown in FIG. 46 and is characterized by Tier 1 reflections at 3.5, 6.9, 12.0°2θ, but also Tier 2 at 9.2, 12.5, 20.2°2θ. A list of 2-theta peaks is provided below:

Peak Table

| Pos. | Rel. Int. |
|---|---|
| [°2Th.] | [%] |
| 3.5 | 100 |
| 6.9 | 6 |
| 9.2 | 1 |
| 12.0 | 4 |
| 12.5 | 1 |
| 15.2 | 1 |
| 19.4 | 1 |
| 20.2 | 1 |

### Example 20. Compound of Formula I, Morpholine Salt

Compound of Formula I, morpholine salt was isolated when about 15 g of compound of Formula I, Form I was combined in a 100 mL bottle with a large stir bar with about 93 mL acetonitrile and about 4.5 g of 2-morpholinoethanol. Mostly a solution with some solids was observed. Seeds were added. Slurry stirred for about 24 hours. The slurry was suction filtered and the cake was rinsed with acetonitrile. The filtered solids were placed in a vacuum oven at 40° C. for about 24 hours. XRPD was taken of the solids.

The XRPD pattern is shown in FIG. 47 and is characterized by Tier 1 reflections at 14.3, 19.0, 22.7 °2θ, but also Tier 2 at 9.0, 14.5, 22.3 °2θ, and Tier 3 at 4.7, 17.2, 26.0° 2θ. A list of 2-theta peaks is provided below:

Peak Table

| Pos. | Rel. Int. |
|---|---|
| [°2Th.] | [%] |
| 4.7 | 15 |
| 9.0 | 47 |
| 9.3 | 32 |
| 11.0 | 15 |
| 13.9 | 18 |
| 14.3 | 100 |
| 14.5 | 69 |
| 17.2 | 40 |
| 18.5 | 28 |
| 19.0 | 49 |
| 19.3 | 28 |
| 22.3 | 47 |
| 22.7 | 50 |
| 26.0 | 31 |
| 28.4 | 24 |
| 29.2 | 19 |
| 29.9 | 28 |
| 36.2 | 11 |

### Example 21. Compound of Formula I, Trans-Ferulic Co-Crystal, Form I

Compound of Formula I, trans-ferulic co-crystal, Form I was first isolated when about 50 mg of compound of Formula I was combined in a vial with about 1 mL methyl tetrahydrofuran or about 1 mL ethyl acetate and trans-ferulic acid. The cap was removed from the vial and the solvent was allowed to evaporate at room temperature. After the solvent was removed, dry solids remained. The solids were characterized by XRPD.

Compound of Formula I, trans-ferulic co-crystal, Form I was also isolated when about 8.5 g of compound of Formula I, Form I, about 4.2 g of ferulic acid, and about 250 mL isopropyl acetate was charged to a 250 mL reactor with the jacket temperature set to about 70° C. A solution was achieved at an internal temperature of about 71° C. The jacket temperature was lowered to about 45° C. and seeds were added. Excess ferulic acid was charged and the system was heat cycled up about 70° C. and down to about 45° C. twice. The system was then cooled to about 20° C. over about 3 hours. The solids were filtered and the cake was rinsed with isopropyl acetate. The solids were dried in a vacuum oven at about 30° C.

The Compound of Formula I, trans-ferulic co-crystal, Form I XRPD pattern is shown in FIG. 51 and is characterized by Tier 1 reflections at 6.4, 25.0, 16.3°2θ, but also Tier 2 at 24.2, 28.8, 8.7°2θ, and Tier 3 at 21.3, 26.9, 22.7°2θ. A list of 2-theta peaks is provided below:

Peak Table

| Pos. | Rel. Int. |
|---|---|
| [°2Th.] | [%] |
| 6.4 | 100 |
| 7.6 | 9 |
| 8.7 | 23 |
| 10.7 | 14 |
| 13.7 | 9 |
| 14.2 | 11 |
| 15.1 | 14 |
| 16.3 | 38 |
| 18.9 | 10 |
| 19.5 | 6 |
| 21.3 | 20 |
| 22.7 | 15 |
| 24.2 | 31 |
| 25.0 | 74 |
| 26.9 | 19 |
| 28.0 | 7 |
| 28.8 | 28 |

The DSC curve is shown in FIG. 52 and displays an endothermic transition at about 139° C. and an endothermic transition at about 180° C. The TGA curve is shown in FIG. 53 and indicates that the phase loses mass starting at about 115° C. The DVS curve is shown in FIG. 54 and the data indicate that the form absorbs less than about 0.35% of water up to 90% RH at 25° C. The material was found to not have changed form post experiment.

### Example 22. Compound of Formula I, Trans-Ferulic Co-Crystal, Form II

Compound of Formula I, trans-ferulic co-crystal, Form II was isolated when about 100 g of compound of Formula I, Form I was combined with about 1.1 equiv. (66 g) ferulic acid in a reactor. About 3000 mL of isopropyl acetate was added. The reactor temperature was increased to about 80° C. and a solution was observed. The reactor was cooled to 45° C. over about 1 hour. Seed was charged. Temperature was cycled from about 45° C. to about 20° C. six times. An aliquot was suction filtered and XRPD was taken and found to me a mixture of forms. Temperature was again cycled from about 45° C. to about 20° C. twelve times. About 82 g of solids were isolated. The reactor was rinsed with the liquors to recover the remaining solids. The isopropyl acetate was distilled portion wise down to about 1000 mL. The slurry was heat cycled from about 45° C. to about 20° C. four times. Slurry stirred for about 24 hours. The slurry was heat cycled from about 45° C. to about 20° C. six times. The slurry stirred for about 24 hours at about 45° C. The slurry was heated to about 80° C. About 15.7 g of compound of Formula I, Form I was added to bring the components back to a 1:1.5 Formula I:trans ferulic acid ratio. A solution was observed. Reactor temperature was set to about 45° C. Seed was charged. Immobile slurry formed. About 150 mL isopropyl acetate was added. Slurry stirred for about 3 hours at 45° C. The slurry was heat cycled from about 45° C. to about 20° C. six times. Solids were suction filtered and characterized by XRPD.

The Compound of Formula I, trans-ferulic co-crystal, Form II XRPD pattern is shown in FIG. 55 and is characterized by Tier 1 reflections at 4.7, 5.9, 25.9°2θ, but also Tier 2 at 15.7, 18.9, 24.4°2θ, and Tier 3 at 4.3, 9.3, 14.4° 2θ. A list of 2-theta peaks is provided below:

Peak Table

| Pos. | |
|---|---|
| [°2Th.] | Rel. Int. |
| 4.3 | 20 |
| 4.7 | 100 |
| 5.9 | 63 |
| 9.3 | 19 |
| 11.7 | 17 |
| 14.4 | 21 |
| 15.7 | 45 |
| 16.7 | 8 |
| 18.9 | 22 |
| 19.5 | 15 |
| 21.3 | 6 |
| 23.5 | 16 |
| 24.4 | 37 |
| 25.9 | 60 |
| 27.4 | 6 |
| 28.9 | 13 |
| 30.0 | 11 |

The DSC curve is shown in FIG. 56 and displays an endothermic transition at about 136° C., 153° C., and 222° C. and an exothermic transition at about 290° C. The TGA curve is shown in FIG. 57 and indicates that the phase is unsolvated.

### Example 23. Compound of Formula I, Tromethamine Co-Crystal

Compound of Formula I, tromethamine co-crystal was isolated when about 8.8 g of compound of Formula I, Form I, about 2.6 g of tromethamine, and about 150 mL isopropanol was charged to a reactor and was agitated at 500 rpm. An additional about 55 mL isopropanol was charged to create a mobile slurry. The slurry stirred overnight. Solids were vacuum filtered and the cake was rinsed with isopropanol. The solids were dried in vacuum oven at 40° C. overnight.

The Compound of Formula I, tromethamine co-crystal XRPD pattern is shown in FIG. 58 and is characterized by Tier 1 reflections at 6.8, 20.4, 27.3°2θ, but also Tier 2 at 16.4, 25.5, 17.3°2θ, and Tier 3 at 21.6, 12.6, 23.7°2θ. A list of 2-theta peaks is provided below:

Peak Table

| Pos. | Rel. Int. |
|---|---|
| [°2Th.] | [%] |
| 6.2 | 5 |
| 6.8 | 100 |
| 12.6 | 3 |
| 14.3 | 1 |
| 16.4 | 12 |
| 17.3 | 6 |
| 20.4 | 24 |
| 21.6 | 6 |
| 23.7 | 3 |
| 25.5 | 7 |
| 26.8 | 3 |
| 27.3 | 15 |
| 32.9 | 2 |
| 37.3 | 3 |

The DSC curve is shown in FIG. 59 and displays an endothermic transition at about 63° C. The TGA curve is shown in FIG. 60 and indicates that the phase loses mass in multiples stages starting at about 50° C. and another stage starting at about 140° C.

### Example 24. Compound of Formula I, L-Arginine Salt Form I

About 83 mg of the compound of Formula I was combined with about 59 mg L-arginine in a 4 mL vial with 1 mL acetone. A suspension was observed. About 1 mL THE was added. A sample was suction filtered and XRPD data was collected. Its XRPD pattern is shown in FIG. 48 and is characterized by Tier 1 reflections at 15.0, 23.3, 27.7° 2θ, but also Tier 2 at 19.4, 24.6, 29.9°2θ, but also Tier 3 at 11.3, 16.7, 22.7°2θ. A list of 2-theta peaks is provided below:

Peak Table

| Pos. | Rel. Int. |
|---|---|
| [°2Th.] | [%] |
| 11.3 | 8 |
| 15.0 | 54 |
| 16.0 | 7 |
| 16.7 | 12 |
| 17.4 | 8 |
| 18.3 | 4 |
| 19.4 | 35 |
| 20.8 | 8 |
| 22.7 | 13 |
| 23.3 | 100 |
| 24.6 | 15 |
| 27.7 | 58 |
| 28.7 | 14 |
| 29.9 | 15 |
| 32.7 | 11 |
| 33.6 | 4 |
| 36.1 | 6 |

### Example 25. Compound of Formula I, L-Arginine Salt Form II

About 439 mg of the compound of Formula I was combined with about 185 mg L-arginine and about 5 mL ethanol in a 20 mL vial with no stir bar. Sample was placed on nutating mixer overnight. A sample was suction filtered and XRPD data was collected. Its XRPD pattern is shown in FIG. 49 and is characterized by Tier 1 reflections at 6.8, 14.5, 20.5° 2θ, but also Tier 2 at 8.9, 11.8, 17.2° 2θ.

Peak Table

| Pos. | Rel. Int. |
|---|---|
| [°2Th.] | [%] |
| 6.8 | 100 |
| 8.9 | 3 |
| 10.5 | 2 |
| 11.8 | 4 |
| 12.9 | 3 |
| 14.5 | 6 |
| 17.2 | 7 |
| 17.9 | 3 |
| 19.2 | 2 |
| 20.5 | 11 |
| 22.1 | 4 |
| 24.4 | 2 |
| 25.5 | 2 |

### Example 26. Compound of Formula I, L-Arginine Salt Form III

The compound of Formula I, L-Arginine Salt Form II was dried in a vacuum oven at room temperature overnight. XRPD was collected from the dried solids. Its XRPD pattern is shown in FIG. 50 and is characterized by Tier 1 reflections at 7.3 and 9.6° 2θ. A list of 2-theta peaks is provided below:

Peak Table

| Pos. | Rel. Int. |
|---|---|
| [°2Th.] | [%] |
| 7.3 | 100 |
| 9.6 | 10 |

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure.

1. A crystalline form of a compound of Formula I:

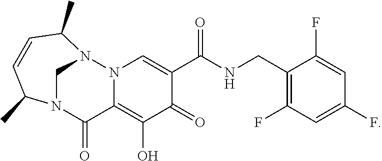

2. The crystalline form of claim 1, characterized by an X-ray powder diffraction (XRPD) pattern comprising 2-θ peaks (±0.2° 2-θ) at 7.0°, 13.9°, and 27.9°.

3. The crystalline form of claim 2, wherein the XRPD pattern further comprises 2-θ peaks (±0.2° 2-θ) at 12.3°, 17.4°, and 24.6°.

4-11. (canceled)

12. The crystalline form of claim 1, characterized by an XRPD pattern comprising 2-θ peaks (±0.2° 2-θ) at 5.7°, 19.9°, and 26.8°.

13. The crystalline form of claim 12, wherein the XRPD pattern further comprises 2-θ peaks (0.2° 2-θ) at 11.4°, 17.6°, and 28.6°.

14-19. (canceled)

20. A salt of a compound of Formula I:

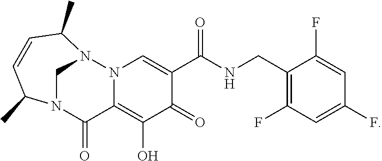

21. The salt of claim 20, wherein the salt is a sodium salt, a potassium salt, a diethylamine salt, an ammonia salt, a calcium salt, a magnesium salt, an N-butylamine salt, a diethanolamine salt, an ethylenediamine salt, a morpholine salt, or a L-arginine salt of the compound of Formula I.

22. A crystalline form of the salt of claim 21, wherein the crystalline form is characterized by an XRPD pattern comprising 2-θ peaks (±0.2° 2-θ) at 13.1°, 14.8°, and 26.8°; wherein the salt is the sodium salt.

23-31. (canceled)

32. A crystalline form of the salt of claim 21, wherein the crystalline form is characterized by an XRPD pattern comprising 2-θ peaks (±0.2° 2-θ) at 5.3°, 7.1°, and 13.4°; wherein the salt is the sodium salt.

33-38. (canceled)

39. A crystalline form of the salt of claim 21, wherein the crystalline form is characterized by an XRPD pattern comprising 2-θ peaks (±0.2° 2-θ) at 7.9°, 8.8°, and 23.1°; wherein the salt is the sodium salt.

40. (canceled)

41. (canceled)

42. A crystalline form of the salt of claim 21, wherein the crystalline form is characterized by an XRPD pattern comprising 2-θ peaks (±0.2° 2-θ) at 5.3°, 6.4°, and 12.8°; wherein the salt is the sodium salt.

43-46. (canceled)

47. A crystalline form of the salt of claim 21, wherein the crystalline form is characterized by an XRPD pattern comprising 2-θ peaks (±0.2° 2-θ) at 7.1°, 20.1°, and 25.8°; wherein the salt is the potassium salt.

48-56. (canceled)

57. A crystalline form of the salt of claim 21, wherein the crystalline form is characterized by an XRPD pattern comprising 2-θ peaks (±0.2° 2-θ) at 6.8°, 30.7°, and 31.4°; wherein the salt is the potassium salt.

58-61. (canceled)

62. A crystalline form of the salt of claim 21, wherein the crystalline form is characterized by an XRPD pattern comprising 2-θ peaks (±0.2° 2-θ) at 5.2°, 13.2°, and 13.7°; wherein the salt is the potassium salt.

63-66. (canceled)

67. A crystalline form of the salt of claim 21, wherein the crystalline form is characterized by an XRPD pattern comprising any three °2-θ peaks (±0.2° 2-θ) selected from 9.7°, 19.5° and 20.5°; wherein the salt is the diethylamine salt.

68-76. (canceled)

77. A crystalline form of the salt of claim 21, wherein the crystalline form is characterized by an XRPD pattern comprising any three °2-θ peaks (±0.2° 2-θ) selected from 6.9°, 12.1°, and 12.5°; wherein the salt is the ammonia salt.

78-87. (canceled)

88. A crystalline form of the salt of claim 21, characterized by an XRPD pattern substantially as set forth in FIG. 34; wherein the salt is the calcium salt.

89. (canceled)

90. (canceled)

91. A crystalline form of the salt of claim 21, wherein the crystalline form is characterized by an XRPD pattern comprising 2-θ peaks (±0.2° 2-θ) at 7.1°, 8.5°, and 11.7°; wherein the salt is the calcium salt.

92. (canceled)

93. (canceled)

94. A crystalline form of the salt of claim 21, wherein the crystalline form is characterized by an XRPD pattern comprising 2-θ peaks (±0.2° 2-θ) at 6.3°, 7.2°, and 25.5°; wherein the salt is the calcium salt.

95-97. (canceled)

98. A crystalline form of the salt of claim 21, wherein the XRPD pattern is substantially as set forth in FIG. 38; wherein the salt is the magnesium salt.

99. (canceled)

100. (canceled)

101. A crystalline form of the salt of claim 21, wherein the crystalline form is characterized by an XRPD pattern comprising 2-θ peaks (±0.2° 2-θ) at 7.0°, 19.8°, and 24.3°; wherein the salt is the magnesium salt.

102-104. (canceled)

105. A crystalline form of the salt of claim 21, wherein the crystalline form is characterized by an XRPD pattern comprising 2-θ peaks (±0.2° 2-θ) at 5.8°, 8.0°, and 10.7°; wherein the salt is the N-butylamine salt.

106-114. (canceled)

115. A crystalline form of the ne salt of claim 21, wherein the crystalline form is characterized by an XRPD pattern comprising 2-θ peaks (±0.2° 2-θ) at 6.1°, 18.4°, and 19.5°; wherein the salt is the diethanolamine salt.

116-119. (canceled)

120. A crystalline form of the salt of claim 21, wherein the crystalline form is characterized by an XRPD pattern comprising 2-θ peaks (±0.2° 2-θ) at 3.5°, 6.9°, and 12.0°; wherein the salt is the ethylenediamine salt.

121-123. (canceled)

124. A crystalline form of the salt of claim 21, wherein the crystalline form is characterized by an XRPD pattern comprising 2-θ peaks (±0.2° 2-θ) at 14.3°, 19.0°, and 22.7°; wherein the salt is the morpholine salt.

125-128. (canceled)

129. A crystalline form of the salt of claim 21, wherein the crystalline form is characterized by an XRPD pattern comprising 2-θ peaks (±0.2° 2-θ) at 15.0°, 23.3°, and 27.7°; wherein the salt is the L-arginine salt.

130-132. (canceled)

133. A crystalline form of the salt of claim 21, wherein the crystalline form is characterized by an XRPD pattern comprising 2-θ peaks (±0.2° 2-θ) at 6.8°, 14.5°, and 20.5°; wherein the salt is the L-arginine salt.

134. (canceled)

135. (canceled)

136. A crystalline form of the salt of claim 21, wherein the crystalline form is characterized by an XRPD pattern comprising 2-θ peaks (0.2° 2-θ) at 7.3° and 9.6°; wherein the salt is the L-arginine salt.

137. (canceled)

138. A co-crystal of a compound of Formula I:

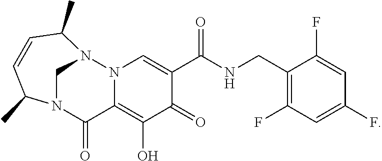

139. The co-crystal of claim 138, wherein the co-crystal is a trans-ferulic or a tromethamine co-crystal.

140. A crystalline form of the co-crystal of claim 139, characterized by an XRPD pattern comprising 2-θ peaks (±0.2° 2-θ) at 6.4°, 16.3°, and 25.0°; wherein the co-crystal is the trans-ferulic co-crystal.

141-148. (canceled)

149. A crystalline form of the co-crystal of claim 139, characterized by an XRPD pattern comprising 2-θ peaks (±0.2° 2-θ) at 4.7°, 5.9°, and 25.9°; wherein the co-crystal is the trans-ferulic co-crystal.

150-157. (canceled)

158. A crystalline form of the co-crystal of claim 139, characterized by an XRPD pattern comprising 2-θ peaks (±0.2° 2-θ) at 6.8°, 20.4°, and 27.3°; wherein the co-crystal is the tromethamine co-crystal.

159-201. (canceled)

202. A pharmaceutical composition comprising a therapeutically effective amount of the crystalline form of claim 1, and a pharmaceutically acceptable carrier or excipient.

203. A pharmaceutical composition comprising a therapeutically effective amount of the salt of claim 20, and a pharmaceutically acceptable carrier or excipient.

204. A pharmaceutical composition comprising a therapeutically effective amount of the co-crystal of claim 138, and a pharmaceutically acceptable carrier or excipient.

205. A method of treating an HIV infection in a human having or at risk of having the infection, comprising administering to the human a therapeutically effective amount of the crystalline form of claim 1.

206. A method of treating an HIV infection in a human having or at risk of having the infection, comprising administering to the human a therapeutically effective amount of the salt of claim 20.

207. A method of treating an HIV infection in a human having or at risk of having the infection, comprising administering to the human a therapeutically effective amount of the co-crystal of claim 138.
